(19) United States
(12) Patent Application Publication
(10) Pub. No.: US 2019/0169235 A1
(43) Pub. Date: Jun. 6, 2019

(54) MODIFIED PROTEIN ENCODING SEQUENCES HAVING INCREASED RARE HEXAMER CONTENT

(71) Applicant: The Research Foundation for the State University of New York, Albany, NY (US)

(72) Inventors: Bruce Futcher, Stony Brook, NY (US); Justin Gardin, Port Jefferson Station, NY (US); Steven Skiena, Setauket, NY (US); Alisa Yurovsky, Stony Brook, NY (US); Eckard Wimmer, East Setauket, NY (US); Steffen Mueller, Kings Point, NY (US)

(21) Appl. No.: 15/773,685

(22) PCT Filed: Nov. 7, 2016
(86) PCT No.: PCT/US2016/060840

§ 371 Date: May 4, 2018

Related U.S. Application Data
(60) Provisional application No. 62/251,320, filed on Nov. 5, 2015.

(51) Int. Cl. C07K 14/005; C12N 7/00

(52) U.S. Cl. CPC C07K 14/005; C12N 2770/32622; C12N 2770/32621; C12N 7/00

(57) ABSTRACT

This invention provides a modified protein encoding sequence containing nucleotide substitutions at multiple locations in the protein encoding sequence, wherein the substitutions introduce rare hexamers. These hexamers may be Frame Dependent, or depleted in only the reading frame, or Frame Independent, or depleted in all three frames. Modified protein encoding se quences of the present invention may include modified viruses useful for vaccines.

### CROSS REFERENCE TO RELATED APPLICATIONS

This application claims the benefit of priority to U.S. Application No. 62/251,320, filed Nov. 5, 2015, which is incorporated herein by reference in its entirety.

### FEDERAL FUNDING

This invention was made with government support under Grant Nos. GM119175 and GM098400 awarded by the National Institutes of Health. The government has certain rights in the invention.

### FIELD OF THE INVENTION

The present invention relates to the creation of modified protein encoding sequences containing a plurality of nucleotide substitutions. The nucleotide substitutions result from the exchange of codons for other synonymous codons and/or codon rearrangement to insert particular rare hexamers. These modified protein encoding sequences may include modified viruses useful for vaccines.

### BACKGROUND OF THE INVENTION

Because the genetic code uses 61 codons to encode only 20 amino acids, there are a tremendous number of ways to encode any given protein. His3, a 220 amino acid yeast protein, can be encoded in ˜10¹⁰⁸ ways, many more than the number of atoms in the known universe (˜10⁸⁰). However, analysis of coding regions shows that not all possible encodings are equally used. Instead, there are biases such that some kinds of encodings are used much more often than others. The best known is “codon bias” or “codon usage”, the tendency to use some synonymous codons more than others (Quax et al., 2015). For instance, human genes use the leucine codon CTG 40% of the time, but use the synonymous CTA only 7% of the time. The frequently-used codons correspond to more highly expressed tRNAs, while the rarely-used codons correspond to poorly expressed tRNAs, but the cause-and-effect relationship behind this correlation is unclear. Although codon bias has been known for decades, the actual mechanism by which a poor codon usage attenuates gene expression is still unknown.

An equally pervasive and important encoding bias is “codon pair bias” (“CPB”) (Gutman et al., 1989). This is the tendency for certain pairs of adjacent codons to be depleted or enriched after normalizing for codon usage. All examined organisms have highly significant codon pair biases in their coding regions. For instance, in yeast, the LeuArg codon pair CUU AGG is used much less often in coding regions than expected, while the LeuArg pair UUG AGG is used much more often than expected (FIG. 1), after taking into consideration the usage of each relevant codon. Codon pair bias is significant in every genome that has been examined

Recently, the inexpensive synthesis of long DNA enabled the de novo synthesis of viruses highly enriched with depleted codon pairs. These viruses include poliovirus (Coleman et al., 2008), influenza virus (Yang et al., 2013; Mueller et al., 2010), and dengue virus (Shen et al., 2015). All such viruses are highly attenuated, in some cases to inviability. The degree of attenuation is correlated with the number of depleted codon pairs. Thus, a negative codon pair bias is somehow functionally important, at least for viruses. Viruses attenuated in this way show no reversion, and are being considered as candidate vaccines.

Despite the success of this approach, no mechanism is known for this attenuation, and there is no obvious facet of mRNA processing, or mRNA translation, or any other aspect of gene expression that would seem to account for such attenuation. A better understanding of how encoding biases cause attenuation would allow more precise control and allow greater predictably in designing attenuated protein encoding sequences, such as attenuated viruses for vaccines.

### SUMMARY OF THE INVENTION

In one aspect, the present disclosure provides a modified protein encoding sequence comprising a polynucleotide sequence derived from a target protein encoding sequence, wherein the modified protein encoding sequence encodes a polypeptide having substantially the same amino acid sequence as the polypeptide encoded by the target protein encoding sequence and comprises a plurality of additional hexamers selected from one or more of the group consisting SEQ ID NO:19 to SEQ ID NO:418 compared to the target protein encoding sequence. In some embodiments, the plurality of hexamers comprises frame independent hexamers. In other embodiments, the plurality of hexamers comprises frame dependent hexamers.

In some embodiments, the modified protein encoding sequence comprises about 50 additional hexamers selected from one or more of the group consisting SEQ ID NO:19 to SEQ ID NO:418 compared to the target protein encoding sequence. In other embodiments, the modified protein encoding sequence comprises about 75 additional hexamers selected from one or more of the group consisting SEQ ID NO:19 to SEQ ID NO:418 compared to the target protein encoding sequence. In some embodiments, the modified protein encoding sequence comprises about 100 additional hexamers selected from one or more of the group consisting SEQ ID NO:19 to SEQ ID NO:418 compared to the target protein encoding sequence.

In some embodiments, the modified protein encoding sequence comprises more than about 50 hexamers. In other embodiments, the modified protein encoding sequence comprises more than about 100 hexamers.

In some embodiments, the modified protein encoding sequence has reduced expression compared to the target protein encoding sequence. In some embodiments, the modified protein encoding sequence has reduced expression in mammalian cells compared to the unmodified protein encoding sequence. In some embodiments, the modified protein encoding sequence has reduced expression in human cells compared to the unmodified protein encoding sequence.

In some embodiments, the modified protein encoding sequence has synonymous codons in a rearranged order compared to the target protein encoding sequence. In some embodiments, the hexamers are introduced by rearranging synonymous codons of the target protein encoding sequence. In other embodiments, the hexamers are introduced by substituting synonymous codons of the target protein encoding sequence.

In some embodiments, the target protein encoding sequence encodes a viral protein. In some embodiments, the present disclosure also provides a modified virus comprising a modified protein encoding sequence, wherein the target protein encoding sequence encodes a viral protein.

In another aspect, the present disclosure provides a method for reducing the expression of a target protein comprising introducing into the target protein encoding sequence a plurality of hexamers selected from one or more of the group consisting SEQ ID NO: 19 to SEQ ID NO: 418 without altering (or without significantly altering) the polypeptide sequence encoded by the target protein encoding sequence. In some embodiments, the plurality of hexamers comprises frame dependent hexamers. In other embodiments, the plurality of hexamers comprises frame independent hexamers.

In some embodiments, greater than about 50 hexamers are introduced into the target protein encoding sequence. In other embodiments, greater than about 100 hexamers are introduced into the target protein encoding sequence.

In some embodiments, hexamers are introduced by rearranging synonymous codons. In other embodiments, hexamers are introduced by substituting synonymous codons.

In some embodiments, the target protein encoding sequence is a viral gene.

In another aspect, the present disclosure provides a modified protein encoding sequence comprising a polynucleotide sequence derived from a target protein encoding sequence, wherein the modified protein encoding sequence encodes a polypeptide having substantially the same amino acid sequence as the polypeptide encoded by the target protein encoding sequence and comprises at least one of: a plurality of frame dependent hexamers each having a frame dependent score less than about -0.51 and a plurality of frame independent hexamers each having a frame independent score less than about −0.33. In some embodiments, the plurality of hexamers comprises frame independent hexamers. In other embodiments, the plurality of hexamers comprises frame dependent hexamers.

In some embodiments, the modified protein encoding sequence comprises about 50 additional hexamers selected from one or more of the group consisting SEQ ID NO:19 to SEQ ID NO:418 compared to the target protein encoding sequence. In other embodiments, the modified protein encoding sequence comprises about 75 additional hexamers selected from one or more of the group consisting SEQ ID NO:19 to SEQ ID NO:418 compared to the target protein encoding sequence. In some embodiments, the modified protein encoding sequence comprises about 100 additional hexamers selected from one or more of the group consisting SEQ ID NO:19 to SEQ ID NO:418 compared to the target protein encoding sequence.

In some embodiments, the modified protein encoding sequence comprises more than about 50 hexamers. In other embodiments, the modified protein encoding sequence comprises more than about 100 hexamers.

In some embodiments, the modified protein encoding sequence has reduced expression compared to the target protein encoding sequence. In some embodiments, the modified protein encoding sequence has reduced expression in mammalian cells compared to the unmodified protein encoding sequence. In some embodiments, the modified protein encoding sequence has reduced expression in human cells compared to the unmodified protein encoding sequence.

In some embodiments, the modified protein encoding sequence has synonymous codons in a rearranged order compared to the target protein encoding sequence. In some embodiments, the hexamers are introduced by rearranging synonymous codons of the target protein encoding sequence. In other embodiments, the hexamers are introduced by substituting synonymous codons of the target protein encoding sequence.

In some embodiments, the target protein encoding sequence encodes a viral protein. In some embodiments, the present disclosure also provides a modified virus comprising a modified protein encoding sequence, wherein the target protein encoding sequence encodes a viral protein.

### BRIEF DESCRIPTION OF THE FIGURES

FIG. 1. A. Changing codon pairs by shuffling synonymous Leu codons UUG and CUU. B, C. Two LYS2 and two HIS3 codon-pair bias (CPB) deoptimized genes compared to their wild-type (WT) and scrambled (SCR) alleles. D. A small segment of the DNA sequence of WT, CPB deoptimized, and scrambled HIS3 alleles. Asterisks indicate residues conserved in all three alleles.

FIG. 2. Expression analysis. Western (top) and Northern (bottom) analysis of wild-type, scrambled (SCR) and codon-pair deoptimized alleles of LYS2. dlys2-4 is a deoptimized allele derived by subcloning part of dlys2-1, and is weakly Lys+. FDF1 is a strongly deoptimized allele (see below) and is Lys−. FDF2,3 is a non-deoptimized control for FDF1 (see below). Arp7 and ACT1 are loading controls.

FIG. 3. Northern analysis. A. Northern analysis comparing mRNA levels of WT HIS3, two biological replicates each of the CPB deoptimized alleles dHIS3-1 and dHIS3-2, and three biological replicates of the scramble HIS3 allele (HIS3-scr). B. Northern analysis comparing mRNA levels of WT LYS2 (either tagged with the HA epitope or not), scrambled LYS2 (LYS2-scr) (tagged with the HA epitope or not) with two CPB deoptimized alleles, dlys2-2, and dlys2-4-3HA. Loading controls in A and B are the ACT1 mRNA, and two ribosomal RNAs.

FIG. 4. Frame Dependent and Independent hexamers. A. The 25 most-depleted frame dependent and frame independent hexamers after averaging FD and FI scores (see Example 2) over eight organisms (E. coli, S. cerevisiae, S. pombe, A. thaliana, D. melanogaster, C. elegans, D. rerio, H. sapiens). Out-of-frame stop codons are shown in bold. B. Frequency distributions of codon pair scores (x-axis) of different classes of codon pairs in yeast. C. Relative attenuation of alleles of HIS3 revealed by serial dilutions of yeast on -His medium containing different amounts of 3-aminotriazole (3-AT). “FDF1” contains yeast codon-pairs depleted from the reading frame in the reading frame, while “FDF2,3” contains such pairs in the other two frames. “codon” dHIS3 is HIS3 synthesized with the worst possible codon usage; it is comparable to the allele used by Presnyak et al.

FIG. 5. Growth of serial dilutions of attenuated alleles of HIS3 on SC-His with 3-aminotriazole. FDF1-1 and FDF1-2 are attenuated with “Frame Dependent” hexamers in frame 1 (where those hexamers are naturally depleted), while FDF23-1 and FDF23-2 are control genes with “Frame Dependent” hexamers in frames 2 and 3 (where they are not naturally depleted). FIF1-1 and FIF1-2 are attenuated with “Frame Independent” hexamers in frame 1 (where those hexamers are naturally depleted), while FIF23-1 and FIF23-2 have the Frame Independent hexamers in frames 2 and 3 (where, these being frame-independent hexamers, they are also naturally depleted). “Codon Deopt” is an allele of HIS3 with the worst possible codon usage, while “Codon Opt” has the best possible codon usage. HIS3 and his3 are wild-type and deletion controls, respectively.

FIG. 6. Polysome profiles and ribosome footprinting. A. A diploid cell carrying wild-type HIS3 on one chromosome, and the attenuated allele HIS3-FDF1-5 on the other chromosome was grown and processed for polysome profiling. A single sucrose gradient was run, and fractions were taken (small numbers are the top (light end) of the gradient). Each fraction was analyzed for amounts of the HIS3 or HIS3-FDF1-5 mRNAs using qRT-PCR, and using primers specific for either HIS3 or HIS3-FDF1-5. The normalized ratio of HIS3 or HIS3-FDF1-5 mRNA to a spike-in control is reported (see Example 4). Peaks of mRNA correspond with polysome peaks (not shown). The rightward shift of the HIS3-FDF1-5 profile indicates that these mRNAs carry on average one ribosome more than the WT HIS3 mRNAs (about 3.5 ribosomes versus about 2.5 ribosomes per mRNA). B. The same diploid strain as above was processed for ribosome profiling (Methods and Materials) and the number of ribosome footprints per mRNA for HIS3 and HIS3-FDF1-5 is reported. The larger number of footprints for HIS3-FDF1-5 suggests that the FDF1-5 mRNA carries more ribosomes than the WT mRNA, consistent with part A above. This is consistent with slow translational elongation. HIS3-FDF1-5 is only mildly attenuated compared to some of the other FDF1 alleles; more severely attenuated alleles could not be assayed because the amounts of mRNA (and so the number of footprints, and the amount of mRNA in polysome gradients) were too small.

FIG. 7. A, B. Yeast growth assays. 3-fold serial dilutions of the indicated mutant yeast strains were grown on YPD or -his medium with the indicated amounts of the His3 inhibitor 3-aminotriazole (3-AT). “FDF1” has Frame Dependent pairs in frame 1; “FDF23” has Frame Dependent pairs in frames 2 and 3; “Codon” has the worst possible codon usage. “Δ” indicates deletion of the entire HIS3 locus. C. Northern analysis showing abundance of the HIS3 transcript from the indicated wild-type (+) or mutant (Δ) strains. “FDF1” is a codon pair deoptimized allele with Frame Dependent pairs in frame 1. “HIS3” is WT HIS3. “SCR1” and “SCR2” are two independent scramble control alleles of HIS3. ACT1 and rRNA are loading controls.

FIG. 8. Yeast growth assays. Low dose gentamicin suppressed rare hexamer attenuation. “FDF1” has Frame Dependent pairs in frame 1; “FDF23” has Frame Dependent pairs in frames 2 and 3. “his3” indicates deletion of the entire HIS3 locus. “HIS3” is WT HIS3.

FIG. 9. A scatterplot of the possible codon pairs with H. sapiens codon pair scores on the x axis and H. sapiens FD scores on the y axis.

### DETAILED DESCRIPTION OF THE INVENTION

Because the genetic code uses 61 codons to encode only 20 amino acids, there are a tremendous number of ways to encode any given protein. However, analysis of coding regions shows that not all possible encodings are equally used. Instead, there are biases such that some kinds of encodings are used much more often than others. The best known is “codon bias” or “codon usage,” the tendency to use some synonymous codons more than others. For instance, in yeast, the Leu codon UGG is used about 28 times per thousand codons, while the Leu codon CUC is used only 5 times per thousand, and this difference is accentuated in highly expressed proteins. The frequently-used codons correspond to more highly expressed tRNAs, while the rarely-used codons correspond to poorly expressed tRNAs, but the cause-and-effect relationship behind this correlation is unclear. Although codon bias has been known for decades, the actual mechanism by which a poor codon usage attenuates gene expression is still unknown.

An equally pervasive and important encoding bias is “codon pair bias” (“CPB”). This is the tendency for certain pairs of adjacent codons to be depleted or enriched in the coding sequences of an organism after normalizing for codon usage. All examined organisms have highly significant codon pair biases in their coding regions. For instance, in yeast, the LeuArg codon pair CUU AGG is used much less often in coding regions than expected, while the LeuArg pair UUG AGG is used much more often than expected, after taking into consideration the usage of each relevant codon. Codon pair bias is significant in every genome that has been examined. WO 2008/121992, which is incorporated herein by reference in its entirety, provides a description of codon pair bias.

A codon pair composed of two codons of three nucleotides each can also be viewed as a single “hexamer” composed of six nucleotides. It has been discovered that certain “rare” “frame-dependent” (FD) hexamers are depleted specifically in the reading frame, while other “frame-independent” (FI) hexamers are depleted in all three frames. These two different types of rare hexamers and their effects on attenuation were investigated, and it was found that introduction of rare FD or FI hexamers attenuated protein expression, although to varying degrees and through seemingly different mechanistic pathways. It was further discovered that the attenuation associated with FD hexamers is because translational quality control pathways such as nonsense mediated decay recognize and destroy mRNAs containing the rare FD hexamers.

Incorporation of these rare hexamers into a protein encoding sequence by substituting synonymous codons and/or by shuffling synonymous codons results in attenuation of expression of the modified protein encoding sequence compared to the target unmodified (e.g., wild type) protein encoding sequence. Accordingly, the present invention relates to a modified protein encoding sequence comprising a polynucleotide sequence derived from a target protein encoding sequence and containing nucleotide substitutions engineered to introduce a plurality of rare hexamers into the protein encoding sequence. In one embodiment, the order of existing codons is changed as compared to a reference (e.g., a wild type) protein encoding sequence, while maintaining the reference amino acid sequence. The change in order alters the occurrence of rare hexamers, and consequently, alters the number of rare hexamers relative to the target protein encoding sequence. The modified protein encoding sequence may comprise rare FD hexamers only, rare FI hexamers only, or a combination of rare FD and rare FI hexamers. In this embodiment, the modified protein sequence is designed to have reduced expression in comparison to the target sequence.

In one embodiment, the modified protein encoding sequence encodes a viral protein, and the present disclosure provides a modified virus comprising the modified protein encoding sequence for the viral protein. These modified viruses are designed to be attenuated as compared to wild type, and may be useful in the preparation of, e.g., vaccines. The modified virus may comprise an increased amount of rare FD hexamers only, rare FI hexamers only, or a combination of rare FD and rare FI hexamers.

This invention also provides a modified host cell line specially isolated or engineered to be permissive for a modified organism that is inviable in a wild type host cell. Since the attenuated organism (e.g., a virus) cannot efficiently grow in normal (wild type) host cells, it is dependent on the specific helper cell line for growth. Various embodiments of the instant modified cell line permit the growth of a modified virus, wherein the genome of said cell line has been altered according to the type of hexamer, (i.e., rare FD or rare FI hexamers) with which the organism has been modified. In one embodiment, the modified cell line may have degraded translation quality control pathways to permit the growth of an organism modified that contains an increased number of rare FD hexamers compared to the unmodified organism.

In another embodiment, the present invention relates to a method for reducing the expression of a target protein comprising introducing into a target protein encoding sequence a plurality of rare hexamers. In some embodiments, the introduction of rare hexamers may be accomplished by rearranging or substituting synonymous codons, such that the resulting sequence has an increased number of rare hexamers relative to the target sequence while still encoding the same, or substantially similar, protein. The method may insert rare FD hexamers only, rare FI hexamers only, or a combination of rare FD and rare FI hexamers.

Encoding Biases

Most amino acids are encoded by more than one codon. See the genetic code in Table 1. For instance, alanine is encoded by four codons: GCU, GCC, GCA, and GCG. Three amino acids (Leu, Ser, and Arg) are encoded by six different codons, while only Trp and Met are each encoded by a single codon (TGG and ATG, respectively). “Synonymous” codons are codons that encode the same amino acid. Thus, for example, CUU, CUC, CUA, CUG, UUA, and UUG are synonymous codons that code for Leu. Synonymous codons are not used with equal frequency. In general, the most frequently used codons in a particular organism are those for which the cognate tRNA is abundant, and the use of these codons enhances the rate and/or accuracy of protein translation. Conversely, tRNAs for the rarely used codons are found at relatively low levels, and the use of rare codons is thought to reduce translation rate and/or accuracy. Thus, to replace a given codon in a nucleic acid by a synonymous but less frequently used codon is to substitute a “deoptimized” codon into the nucleic acid.

TABLE 1
Genetic Code

| | U | C | A | G | |
|---|---|---|---|---|---|
| U | Phe | Ser | Tyr | Cys | U |
| | Phe | Ser | Tyr | Cys | C |
| | Leu | Ser | STOP | STOP | A |
| | Leu | Ser | STOP | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

^{a} The first nucleotide in each codon encoding a particular amino acid is shown in the left-most column; the second nucleotide is shown in the top row; and the third nucleotide is shown in the right-most column.

Codon Bias

Whereas most amino acids can be encoded by multiple different codons, not all codons are used equally frequently: some codons are “rare” codons, whereas others are “frequent” codons. As used herein, a “rare” codon is one of at least two synonymous codons encoding a particular amino acid that is present in an mRNA at a significantly lower frequency than the most frequently used codon for that amino acid. Thus, the rare codon may be present at about a 2-fold lower frequency than the most frequently used codon. Preferably, the rare codon is present at least a 3-fold, more preferably at least a 5-fold, lower frequency than the most frequently used codon for the amino acid. Conversely, a “frequent” codon is one of at least two synonymous codons encoding a particular amino acid that is present in an mRNA at a significantly higher frequency than the least frequently used codon for that amino acid. The frequent codon may be present at about a 2-fold, preferably at least a 3-fold, more preferably at least a 5-fold, higher frequency than the least frequently used codon for the amino acid. For example, human genes use the leucine codon CTG 40% of the time, but use the synonymous CTA only 7% of the time (see Table 2). Thus, CTG is a frequent codon, whereas CTA is a rare codon. Roughly consistent with these frequencies of usage, there are 6 copies in the genome for the gene for the tRNA recognizing CTG, whereas there are only 2 copies of the gene for the tRNA recognizing CTA. Similarly, human genes use the frequent codons TCT and TCC for serine 18% and 22% of the time, respectively, but the rare codon TCG only 5% of the time. TCT and TCC are read, via wobble, by the same tRNA, which has 10 copies of its gene in the genome, while TCG is read by a tRNA with only 4 copies in the genome. Those mRNAs that are very actively translated are strongly biased to use only the most frequent codons. This includes genes for ribosomal proteins and glycolytic enzymes. On the other hand, mRNAs for relatively non-abundant proteins may use the rare codons.

TABLE 2
Codon usage in Homo sapiens (source: http://www.kazusa.or.jp/codon/)

| Amino Acid | Codon | Number | /1000 | Fraction |
|---|---|---|---|---|
| Gly | GGG | 636457.00 | 16.45 | 0.25 |
| Gly | GGA | 637120.00 | 16.47 | 0.25 |
| Gly | GGT | 416131.00 | 10.76 | 0.16 |
| Gly | GGC | 862557.00 | 22.29 | 0.34 |
| Glu | GAG | 1532589.00 | 39.61 | 0.58 |
| Glu | GAA | 1116000.00 | 28.84 | 0.42 |
| Asp | GAT | 842504.00 | 21.78 | 0.46 |
| Asp | GAC | 973377.00 | 25.16 | 0.54 |
| Val | GTG | 1091853.00 | 28.22 | 0.46 |
| Val | GTA | 273515.00 | 7.07 | 0.12 |
| Val | GTT | 426252.00 | 11.02 | 0.18 |
| Val | GTC | 562086.00 | 14.53 | 0.24 |
| Ala | GCG | 286975.00 | 7.42 | 0.11 |
| Ala | GCA | 614754.00 | 15.89 | 0.23 |
| Ala | GCT | 715079.00 | 18.48 | 0.27 |
| Ala | GCC | 1079491.00 | 27.90 | 0.40 |
| Arg | AGG | 461676.00 | 11.93 | 0.21 |
| Arg | AGA | 466435.00 | 12.06 | 0.21 |
| Ser | AGT | 469641.00 | 12.14 | 0.15 |
| Ser | AGC | 753597.00 | 19.48 | 0.24 |
| Lys | AAG | 1236148.00 | 31.95 | 0.57 |
| Lys | AAA | 940312.00 | 24.30 | 0.43 |
| Asn | AAT | 653566.00 | 16.89 | 0.47 |
| Asn | AAC | 739007.00 | 19.10 | 0.53 |
| Met | ATG | 853648.00 | 22.06 | 1.00 |
| Ile | ATA | 288118.00 | 7.45 | 0.17 |
| Ile | ATT | 615699.00 | 15.91 | 0.36 |
| Ile | ATC | 808306.00 | 20.89 | 0.47 |
| Thr | ACG | 234532.00 | 6.06 | 0.11 |
| Thr | ACA | 580580.00 | 15.01 | 0.28 |
| Thr | ACT | 506277.00 | 13.09 | 0.25 |
| Thr | ACC | 732313.00 | 18.93 | 0.36 |
| Trp | TGG | 510256.00 | 13.19 | 1.00 |
| End | TGA | 59528.00 | 1.54 | 0.47 |
| Cys | TGT | 407020.00 | 10.52 | 0.45 |
| Cys | TGC | 487907.00 | 12.61 | 0.55 |
| End | TAG | 30104.00 | 0.78 | 0.24 |
| End | TAA | 38222.00 | 0.99 | 0.30 |
| Tyr | TAT | 470083.00 | 12.15 | 0.44 |
| Tyr | TAC | 592163.00 | 15.30 | 0.56 |
| Leu | TTG | 498920.00 | 12.89 | 0.13 |
| Leu | TTA | 294684.00 | 7.62 | 0.08 |
| Phe | TTT | 676381.00 | 17.48 | 0.46 |
| Phe | TTC | 789374.00 | 20.40 | 0.54 |
| Ser | TCG | 171428.00 | 4.43 | 0.05 |
| Ser | TCA | 471469.00 | 12.19 | 0.15 |
| Ser | TCT | 585967.00 | 15.14 | 0.19 |
| Ser | TCC | 684663.00 | 17.70 | 0.22 |
| Arg | CGG | 443753.00 | 11.47 | 0.20 |
| Arg | CGA | 239573.00 | 6.19 | 0.11 |
| Arg | CGT | 176691.00 | 4.57 | 0.08 |
| Arg | CGC | 405748.00 | 10.49 | 0.18 |
| Gln | CAG | 1323614.00 | 34.21 | 0.74 |
| Gln | CAA | 473648.00 | 12.24 | 0.26 |
| His | CAT | 419726.00 | 10.85 | 0.42 |
| His | CAC | 583620.00 | 15.08 | 0.58 |
| Leu | CTG | 1539118.00 | 39.78 | 0.40 |
| Leu | CTA | 276799.00 | 7.15 | 0.07 |
| Leu | CTT | 508151.00 | 13.13 | 0.13 |
| Leu | CTC | 759527.00 | 19.63 | 0.20 |
| Pro | CCG | 268884.00 | 6.95 | 0.11 |
| Pro | CCA | 653281.00 | 16.88 | 0.28 |
| Pro | CCT | 676401.00 | 17.48 | 0.29 |
| Pro | CCC | 767793.00 | 19.84 | 0.32 |

The propensity for highly expressed genes to use frequent codons is called “codon bias.” A gene for a ribosomal protein might use only the 20 to 25 most frequent of the 61 codons, and have a high codon bias (a codon bias close to 1), while a poorly expressed gene might use all 61 codons, and have little or no codon bias (a codon bias close to 0). It is thought that the frequently used codons are codons where larger amounts of the cognate tRNA are expressed, and that use of these codons allows translation to proceed more rapidly, or more accurately, or both.

Codon Pair Bias

A distinct feature of coding sequences is their codon pair bias. This is the tendency for certain pairs of adjacent codons to be depleted or enriched after normalizing for codon usage. All examined organisms have highly significant codon pair biases in their coding regions. For instance, in yeast, the LeuArg codon pair CUU AGG is used much less often in coding regions than expected, while the LeuArg pair UUG AGG is used much more often than expected, after taking into consideration the usage of each relevant codon.

Each codon pair can be given a codon pair score (“CPS”), which is:

$$Ln ( \frac{observed occurrences}{expected occurrences} )$$

where observed occurrences are the number of occurrences of that codon pair in all coding regions of the genome, and the expected occurrences are the number expected based on (a) the frequency of the amino acid pair and (b) the frequency of each relevant codon. Because this is a natural log, enriched codon pairs have a positive score, and depleted pairs have a negative score. Using the calculated codon pair score, any coding region k codons in length can then be rated as using as using over- or under-represented codon pairs by taking the average of the codon pair scores, thus giving a codon pair bias (CPB) for the coding region:

$$C P B = {\sum }_{i = 1}^{k} \frac{{CPS}_{i}}{k - 1}$$

Because the calculation for codon pair score includes a normalization for the frequency of each synonymous codon, in principle codon pair bias is, mathematically, completely independent from codon bias. Indeed, there is little or no correlation between a codon pair score, and the frequency of use of each of the two codons it contains. Some depleted codon pairs are composed of two common codons (e.g., GluLys, GUU AAA, codon pair score −0.283), while some enriched codon pairs are composed of two rare codons (SerThr, AGC ACG, codon pair score 0.171). This is possible because enrichment or depletion is calculated compared to expectation based on codon usage, not in absolute terms. That is, the codon pair score is measuring a bias for or against particular adjacent pairs of codons, but taking into account the existing bias for or against those codons individually.

Codon pair scores for eight species (S. cerevisiae, S. pombe, E. coli, C. elegans, D. rerio, D. melanogaster, A. thaliana, and H. sapiens) were generated by bootstrapping the expected hexamer occurrence through many iterations of synonymous codon shuffling for all genes annotated in a given genome. 200 random synonymous shuffles of each gene for each genome was selected to dampen variance caused by small genome size or rare codon occurrence. The codon pair scores for the complete set of 3721 (61²) codon pairs for each of the eight species are provided herewith as Supplemental Table 1.

“Rare” Hexamers

The present disclosure provides, for the first time, two distinct classifications of depleted codon pairs. A codon pair composed of two codons of three nucleotides each can be viewed a single “hexamer” composed of six nucleotides that may occur in any of the three reading frames. That is, a hexamer XXX-XXX may also appear within a coding sequence as nXX-XXX-Xnn or nnX-XXX-XXn. In these other frames, it is usually the case that the hexamer helps to encode other amino acids. For example, the hexamer CUG-CAC encodes LeuHis in frame 1, but would encode ?-Ala-? in frame 2 (xCU-GCA-Cxx) and CysThr in frame 3 (xxC-UGC-ACx).

As used herein, a “Frame Dependent” (FD) hexamer is one that is depleted in the reading frame only. A Frame Dependent Score is calculated according to the following formula:

$$FDscore ( hexamer ) = {CPS}_{Frame 1} ( hexamer ) - \frac{{CPS}_{Frame 2} ( hexamer ) + {CPS}_{Frame 3} ( hexamer )}{2}$$

Frame Dependent scores for hexamers containing an out-of-frame stop (OOFS) codon were altered according to the following formula to allow for the fact that such hexamers are not permissible in one of the three frames:

FDscore(OOFS hexamer)=CPS_{Frame 1}(OOFS hexamer)−CPS_{Frame 2 or 3} (OOPS hemmer)

Using the calculated FD Scores, any coding region of k codons in length can then be rated as using these rare FD hexamers by taking the average of the FD Scores, thus giving an FD bias for the coding region:

$$F D Bias = {\sum }_{i = 1}^{k} \frac{F D {Score}_{i}}{k - 1}$$

As used herein, a “Frame Independent” (FI) hexamer is one that is depleted in all three frames. A Frame Independent Score is calculated according to the following formula:

$$FIscore ( hexamer ) = \frac{\begin{matrix}{CPS}_{Frame 3} ( hexamer ) + \\ {CPS}_{Frame 2} ( hexamer ) + {CPS}_{Frame 3} ( hexamer )\end{matrix}}{3}$$

Hexamers containing out-of-frame stop codons were excluded from Frame Independent score calculation, as they are inherently Frame Dependent.

Using the calculated FI Scores, any coding region of k codons in length can then be rated as using these rare FI hexamers by taking the average of the FI Scores, thus giving an FI bias for the coding region:

$$F I Bias = {\sum }_{i = 1}^{k} \frac{F I {Score}_{i}}{k - 1}$$

Table 3 shows the 100 most-depleted (most negative scoring) Frame Dependent and Frame Independent hexamers after averaging scores over eight organisms (S. cerevisiae, S. pombe, E. coli, C. elegans, D. rerio, D. melanogaster, A. thaliana, and H. sapiens). Table 4 shows the 100 most-depleted (most negative scoring) Frame Dependent and Frame Independent hexamers for H. sapiens. The full set of FD and FI scores for each of the eight species is provided here in Supplemental Tables S2 and S3, respectively. The full set of FD and FI scores averaged across the eight species is provided here in Supplemental Table S4.

TABLE 3

| FD | FD | SEQ ID | FI | FI | SEQ ID |
|---|---|---|---|---|---|
| Hexamer | Scores | NO: | Hexamer | Scores | NO: |
| TCTAGC | -0.88 | 19 | CCCCCC | -1.04 | 119 |
| GCTATG | -0.87 | 20 | GGGGGG | -0.95 | 120 |
| GCTAAG | -0.87 | 21 | ACCCCC | -0.66 | 121 |
| CTCGCT | -0.84 | 22 | GGGGGT | -0.65 | 122 |
| TTCGCT | -0.80 | 23 | CCCCCG | -0.64 | 123 |
| CTCGCA | -0.77 | 24 | CGGGGG | -0.57 | 124 |
| TTCGCA | -0.75 | 25 | CCCCCT | -0.57 | 125 |
| TGCGCT | -0.75 | 26 | GCCCCC | -0.56 | 126 |
| CTCCCA | -0.75 | 27 | CCCCTA | -0.54 | 127 |
| GCTAAC | -0.74 | 28 | CGCGCG | -0.54 | 128 |
| TGTAGC | -0.74 | 29 | CGCCCC | -0.54 | 129 |
| GCTAGC | -0.74 | 30 | GCGCGA | -0.53 | 130 |
| TTTAGG | -0.73 | 31 | CGCGAA | -0.52 | 131 |
| CTCGTG | -0.72 | 32 | TACGTA | -0.51 | 132 |
| TGTAGA | -0.71 | 33 | AGGGGG | -0.51 | 133 |
| TCCGCT | -0.70 | 34 | TCGCGA | -0.50 | 134 |
| CTCCTG | -0.70 | 35 | CGCGTA | -0.49 | 135 |
| GCTACA | -0.70 | 36 | GCGCGC | -0.47 | 136 |
| CTCCAA | -0.69 | 37 | CCCCGC | -0.47 | 137 |
| GCCGCT | -0.69 | 38 | GGGGTA | -0.47 | 138 |
| ATTAGG | -0.68 | 39 | TTTTTT | -0.46 | 139 |
| CATAGA | -0.68 | 40 | GGGGTG | -0.46 | 140 |
| GTCGCT | -0.67 | 41 | AAAAAA | -0.46 | 141 |
| TGTAGG | -0.67 | 42 | CGGTCC | -0.44 | 142 |
| ACCGCT | -0.66 | 43 | ACGCGA | -0.44 | 143 |
| CATAGC | -0.66 | 44 | GGTCCC | -0.44 | 144 |
| TCCGCA | -0.65 | 45 | CGCGAG | -0.44 | 145 |
| GCTAAA | -0.65 | 46 | CGCGAC | -0.43 | 146 |
| TTCGTT | -0.65 | 47 | TACCCC | -0.43 | 147 |
| CATTGG | -0.65 | 48 | TTCGCG | -0.42 | 148 |
| GTCGCA | -0.64 | 49 | CGCGAT | -0.42 | 149 |
| GCCGCA | -0.64 | 50 | CACCCC | -0.41 | 150 |
| GCTACG | -0.64 | 51 | GTCCCC | -0.41 | 151 |
| TGCGGA | -0.64 | 52 | GGGCCC | -0.41 | 152 |
| TCTAAG | -0.64 | 53 | CCCCGA | -0.41 | 153 |
| ATCGCT | -0.64 | 54 | GACCCC | -0.41 | 154 |
| GATAGC | -0.64 | 55 | AGCGCG | -0.41 | 155 |
| AACGCT | -0.63 | 56 | TCCCCC | -0.41 | 156 |
| CACCAA | -0.63 | 57 | CCCGCG | -0.40 | 157 |
| TTCGGA | -0.63 | 58 | GGCGCC | -0.40 | 158 |
| AGTAGG | -0.63 | 59 | ACCCCG | -0.40 | 159 |
| TGCGCA | -0.62 | 60 | GTCCTA | -0.40 | 160 |
| GATTGG | -0.62 | 61 | TACGCG | -0.40 | 161 |
| ACTAAG | -0.62 | 62 | GTCGCG | -0.40 | 162 |
| GCTACC | -0.62 | 63 | GCGCCC | -0.40 | 163 |
| ACCGCA | -0.61 | 64 | GGCCCC | -0.40 | 164 |
| GACGCT | -0.61 | 65 | CGTACG | -0.39 | 165 |
| ACTAGC | -0.61 | 66 | CCCTAT | -0.39 | 166 |
| AGCGCT | -0.60 | 67 | CGCGCA | -0.39 | 167 |
| AGGTGG | -0.60 | 68 | GGGGGA | -0.39 | 168 |
| AGCGCA | -0.60 | 69 | GACGTA | -0.39 | 169 |
| TTTAGC | -0.59 | 70 | CGAACG | -0.39 | 170 |
| CTTAAG | -0.59 | 71 | CCCCCA | -0.39 | 171 |
| GCTAGT | -0.59 | 72 | GTACGT | -0.39 | 172 |
| CACGCT | -0.59 | 73 | CGGGGT | -0.39 | 173 |
| CTCGAA | -0.59 | 74 | TCGCGC | -0.38 | 174 |
| GGCGCT | -0.58 | 75 | CTCGCG | -0.38 | 175 |
| GTCGTG | -0.58 | 76 | TTCGTA | -0.38 | 176 |
| CGGTGG | -0.57 | 77 | GAGGTA | -0.38 | 177 |
| ACTATG | -0.57 | 78 | TCGCGT | -0.38 | 178 |
| GCTAAT | -0.57 | 79 | TTACGT | -0.38 | 179 |
| GCTATC | -0.57 | 80 | CGGCCG | -0.38 | 180 |
| TTTAAG | -0.57 | 81 | AACGCG | -0.38 | 181 |
| CTTATG | -0.56 | 82 | TATACG | -0.37 | 182 |
| AGTAGA | -0.56 | 83 | CGGTCG | -0.37 | 183 |
| TTCGTG | -0.56 | 84 | AGGTAC | -0.37 | 184 |
| TTCGAA | -0.56 | 85 | AGGGGT | -0.37 | 185 |
| ATTAGA | -0.56 | 86 | TGCGCG | -0.37 | 186 |
| TTTAGA | -0.56 | 87 | GCCCCG | -0.37 | 187 |
| AATAGG | -0.56 | 88 | ACCCCT | -0.37 | 188 |
| GGTAAG | -0.55 | 89 | CGTGCG | -0.36 | 189 |
| CATAGG | -0.55 | 90 | AACGTA | -0.36 | 190 |
| ATCGCA | -0.55 | 91 | CCCCGT | -0.36 | 191 |
| GACGCA | -0.55 | 92 | GCGCGT | -0.36 | 192 |
| TCTAGA | -0.55 | 93 | CGTATA | -0.36 | 193 |
| CTGCCA | -0.55 | 94 | GCCCCT | -0.36 | 194 |
| CATAGT | -0.55 | 95 | CTTACG | -0.36 | 195 |
| TTTAAC | -0.55 | 96 | CCGCGA | -0.36 | 196 |
| GTTAGG | -0.54 | 97 | AGCCCC | -0.36 | 197 |
| TCTAAC | -0.54 | 98 | ACGTAC | -0.36 | 198 |
| ATTAGC | -0.54 | 99 | CCGCGG | -0.36 | 199 |
| GGGTGG | -0.53 | 100 | GCCCTA | -0.35 | 200 |
| CTCGTT | -0.53 | 101 | ATACGT | -0.35 | 201 |
| TGCGTT | -0.53 | 102 | GCGGGG | -0.35 | 202 |
| CTCCTC | -0.53 | 103 | GGGTCC | -0.35 | 203 |
| GCTAGG | -0.52 | 104 | CCCCGG | -0.35 | 204 |
| ATTAAC | -0.52 | 105 | CCCCTC | -0.35 | 205 |
| AGTACA | -0.52 | 106 | GTCCGA | -0.35 | 206 |
| CTCCAG | -0.52 | 107 | GGGGGC | -0.35 | 207 |
| TTTAGT | -0.52 | 108 | CCCGTA | -0.34 | 208 |
| GTTATG | -0.52 | 109 | GTTGCG | -0.34 | 209 |
| TACGCT | -0.52 | 110 | ACGCGC | -0.34 | 210 |
| GCCGTG | -0.52 | 111 | CGCATA | -0.34 | 211 |
| TGTAGT | -0.52 | 112 | TCGTAC | -0.34 | 212 |
| CTGTCA | -0.51 | 113 | GGGGTC | -0.34 | 213 |
| TTTTGG | -0.51 | 114 | AACCCC | -0.34 | 214 |
| CACCCA | -0.51 | 115 | GAGGGG | -0.34 | 215 |
| GTCGAA | -0.51 | 116 | CAGGTA | -0.33 | 216 |
| CACCTG | -0.51 | 117 | GTCGTA | -0.33 | 217 |
| AGGTGC | -0.51 | 118 | ACGGGG | -0.33 | 218 |

TABLE 4

| FD | FD | SEQ ID | FI | FI | SEQ ID |
|---|---|---|---|---|---|
| Hexamer | Score | NO: | Hexamer | Score | NO: |
| GCCGCT | -1.92 | 219 | CGCGAA | -1.45 | 319 |
| CTCGAA | -1.86 | 220 | TCGCGA | -1.42 | 320 |
| CTCGCT | -1.85 | 221 | CGATCG | -1.17 | 321 |
| CCCGCT | -1.82 | 222 | CGAACG | -1.12 | 322 |
| CTCGGA | -1.80 | 223 | ACGCGA | -1.12 | 323 |
| GTCGCT | -1.80 | 224 | CGCGAT | -1.10 | 324 |
| GGCGCT | -1.78 | 225 | GCGAAA | -1.10 | 325 |
| TCCGCT | -1.76 | 226 | GCGAAC | -1.02 | 326 |
| ACCGCT | -1.75 | 227 | CGGTCG | -1.02 | 327 |
| TGCGCT | -1.74 | 228 | CGCGTA | -1.01 | 328 |
| GCCGCA | -1.68 | 229 | CGCAAT | -1.00 | 329 |
| CTCGAG | -1.62 | 230 | CGTCGA | -0.98 | 330 |
| CGCGCT | -1.61 | 231 | CCGGTA | -0.98 | 331 |
| CTCGCA | -1.57 | 232 | GTTGCG | -0.97 | 332 |
| GCCGGA | -1.56 | 233 | TCGATC | -0.97 | 333 |
| TGCGGA | -1.56 | 234 | GCGATC | -0.96 | 334 |
| TTCGAA | -1.55 | 235 | TCGCGT | -0.96 | 335 |
| CTCGGT | -1.55 | 236 | TTTCGC | -0.96 | 336 |
| GTCGAA | -1.54 | 237 | ACGATC | -0.94 | 337 |
| TCCGCA | -1.53 | 238 | TTGCGA | -0.93 | 338 |
| GTCGCA | -1.52 | 239 | CAATCG | -0.92 | 339 |
| AGCGCT | -1.51 | 240 | CGACGA | -0.92 | 340 |
| ACCGCA | -1.51 | 241 | GTCGAA | -0.92 | 341 |
| GTCGGA | -1.50 | 242 | CGCGAC | -0.91 | 342 |
| GTCGAG | -1.48 | 243 | CCGATC | -0.91 | 343 |
| TTCGCT | -1.45 | 244 | TCGCAA | -0.91 | 344 |
| CTCGGC | -1.45 | 245 | CGATCA | -0.91 | 345 |
| TTCGGA | -1.43 | 246 | TATACG | -0.90 | 346 |
| CATAGA | -1.42 | 247 | GCGATA | -0.90 | 347 |
| TCCGAA | -1.42 | 248 | CGTACG | -0.90 | 348 |
| TCCGGA | -1.41 | 249 | GGCGAA | -0.89 | 349 |
| TGCGCA | -1.37 | 250 | CGATTG | -0.88 | 350 |
| GGCGCA | -1.36 | 251 | TACGCG | -0.88 | 351 |
| CCCGGA | -1.35 | 252 | CCCCCC | -0.88 | 352 |
| GGCGGA | -1.35 | 253 | TTACGC | -0.88 | 353 |
| ACCGAA | -1.34 | 254 | GCGCGA | -0.88 | 354 |
| CTCGCC | -1.33 | 255 | CTCGCG | -0.87 | 355 |
| CATAGC | -1.33 | 256 | GTCGCG | -0.87 | 356 |
| TTCGTT | -1.33 | 257 | TTCGCG | -0.87 | 357 |
| CCCGCA | -1.33 | 258 | TTTTCG | -0.87 | 358 |
| CCTAGC | -1.30 | 259 | GCGCAA | -0.87 | 359 |
| CACGCT | -1.29 | 260 | CGAAAA | -0.87 | 360 |
| ACCGGA | -1.28 | 261 | GTCGAT | -0.87 | 361 |
| AGCGCA | -1.26 | 262 | CGCATA | -0.87 | 362 |
| GCCGAA | -1.25 | 263 | CGATCC | -0.86 | 363 |
| GTCGGC | -1.25 | 264 | CGATCT | -0.86 | 364 |
| GTCGCC | -1.25 | 265 | CGCAAC | -0.86 | 365 |
| GCTAGG | -1.25 | 266 | CGGTAT | -0.86 | 366 |
| GCCGCC | -1.25 | 267 | ATACGC | -0.85 | 367 |
| CATAGG | -1.25 | 268 | ATTCGC | -0.85 | 368 |
| TCCGCC | -1.24 | 269 | TCGAAC | -0.85 | 369 |
| TCCGTT | -1.24 | 270 | ACGGTA | -0.85 | 370 |
| AACGCT | -1.24 | 271 | ATTGCG | -0.84 | 371 |
| GCTAGC | -1.24 | 272 | AACGCG | -0.84 | 372 |
| CCTAGA | -1.24 | 273 | TCTCGA | -0.84 | 373 |
| TGTAGC | -1.22 | 274 | ACGAAC | -0.84 | 374 |
| CTCGGG | -1.22 | 275 | ACGATA | -0.83 | 375 |
| CCCGTT | -1.22 | 276 | CGATAC | -0.83 | 376 |
| AGCGTT | -1.22 | 277 | ACCGGT | -0.83 | 377 |
| CCCGGT | -1.21 | 278 | CGATTA | -0.83 | 378 |
| CCTAGG | -1.21 | 279 | GGTCGA | -0.83 | 379 |
| AGCGGA | -1.20 | 280 | GCGTAC | -0.82 | 380 |
| GCCGTT | -1.19 | 281 | GACGAA | -0.82 | 381 |
| GACGCT | -1.19 | 282 | GGGGGG | -0.82 | 382 |
| CGCGCA | -1.19 | 283 | CGAACC | -0.82 | 383 |
| TCCGAG | -1.19 | 284 | GTCGTA | -0.81 | 384 |
| GGTAGG | -1.18 | 285 | ATACCG | -0.81 | 385 |
| GTCGTT | -1.18 | 286 | CGGTAC | -0.81 | 386 |
| TGTAGG | -1.16 | 287 | GGCGTA | -0.81 | 387 |
| CCCGAA | -1.16 | 288 | GCGATT | -0.81 | 388 |
| GGCGAA | -1.16 | 289 | ATCGCG | -0.81 | 389 |
| TGTAGA | -1.16 | 290 | CGATAT | -0.80 | 390 |
| CTCGAC | -1.15 | 291 | CGAACT | -0.80 | 391 |
| AGTAGA | -1.15 | 292 | TCGGTA | -0.80 | 392 |
| TTCGCA | -1.15 | 293 | ACGCAA | -0.80 | 393 |
| TTCGGT | -1.14 | 294 | TACCGG | -0.80 | 394 |
| AGTAGG | -1.14 | 295 | TTTACG | -0.79 | 395 |
| GATAGC | -1.14 | 296 | TTGCGC | -0.79 | 396 |
| AGCGAA | -1.14 | 297 | TCGACG | -0.79 | 397 |
| TCCGGT | -1.12 | 298 | ATTTCG | -0.79 | 398 |
| GCCGGT | -1.12 | 299 | GCGGTA | -0.79 | 399 |
| TCCGAT | -1.11 | 300 | AGCGTA | -0.79 | 400 |
| ACCGCC | -1.11 | 301 | GCGTAT | -0.79 | 401 |
| GACGGA | -1.11 | 302 | CCGATA | -0.79 | 402 |
| CTCGAT | -1.11 | 303 | CCGAAC | -0.78 | 403 |
| TGCGTT | -1.10 | 304 | ACGAAA | -0.78 | 404 |
| AGTAGC | -1.10 | 305 | GTCGAC | -0.78 | 405 |
| TACGCT | -1.10 | 306 | ATTACG | -0.78 | 406 |
| CCCGGC | -1.09 | 307 | TTTCGA | -0.77 | 407 |
| GTCGAC | -1.09 | 308 | CATACG | -0.77 | 408 |
| GTCGGT | -1.09 | 309 | CGAAAC | -0.77 | 409 |
| CCCGCC | -1.09 | 310 | CGAACA | -0.77 | 410 |
| CCATGC | -1.08 | 311 | CGTATA | -0.77 | 411 |
| TCTAGC | -1.06 | 312 | ACGCGT | -0.77 | 412 |
| GGTAGA | -1.06 | 313 | GACGTA | -0.77 | 413 |
| TGCGGT | -1.05 | 314 | CTATCG | -0.76 | 414 |
| GGTAGC | -1.05 | 315 | TTGCGT | -0.76 | 415 |
| TGCGAA | -1.05 | 316 | ACGATT | -0.76 | 416 |
| ATCGGA | -1.05 | 317 | TCGATT | -0.76 | 417 |
| CATTGG | -1.05 | 318 | CCGCGA | -0.76 | 418 |

Modified Protein Encoding Sequences Using “Rare” Hexamers

The present invention provides a modified protein encoding sequence derived from a target encoding sequence and comprising a plurality of rare hexamers. As used herein, a “rare” hexamer is one that of the 25, 100, 500, or 1000 most-depleted FD or FI hexamers. In some embodiments, the modified protein encoding sequence comprises a plurality of hexamers selected from Table 2. The most-depleted hexamers may be determined with reference to Supplemental Table S4, or with reference to a specific species provided in Supplemental Tables S2 or S3, or with reference to bioinformatic analysis of the most-depleted hexamers of any other species, calculated as described above. In some embodiments, the “rare” hexamers may comprise hexamers that have FD scores of less than −0.1, less than −0.2, less than −0.3, less than −0.4, less than −0.5, less than −0.6, or less than −0.7. In other embodiments, the “rare” hexamers may comprise hexamers that have FI scores of less than −0.1, less than −0.2, less than −0.3, less than −0.4, or less than −0.5.

In some embodiments, the modified protein encoding sequence rare hexamer content may be described in comparison to the target encoding sequence from which it was derived, and may comprise a polynucleotide sequence derived from a target encoding sequence and comprises at least 5, 10, 25, 50, 75, 100, 250, 500, or 1000 additional rare hexamers when compared to the target encoding sequence. In other embodiments, the modified protein encoding sequence rare hexamer content may be described in absolute terms, and may comprise a polynucleotide sequence derived from a target encoding sequence and comprises at least 10, 20, 30, 40, 50, 60, 70, 80, 90, 100, 250, 500 or 1000 rare hexamers. It is also understood that the number of modifications may be made with reference to the overall length of the target encoding sequence. Accordingly, the level of the defined rare hexamers (e.g., the 25, 100, 500, or 1000 most-depleted FD and/or FI hexamers) in a target encoding sequence may be determined as a percentage of the total number of hexamers in the target encoding sequence, and the modified protein encoding sequence having an increased percentage of rare hexamers (FD and/or FI) compared to the target encoding sequence. This may be deteremined as an increase in the percentage of rare hexamers compared to the total number of hexamers, or an increase in the percentage of rare hexamers itself. For example, if a target encoding sequence comprises 5% rare hexamers, a modified protein encoding sequence of the present disclosure having an increased percentage of rare hexamers may, as a non-limiting example, have 10% rare hexamers, or a rare hexamer percentage increase of 100%.

In some embodiments, the modified protein encoding sequence may comprise 0.1%, 1%, 5%, 10%, 25%, or 50% rare hexamers. In other embodiments, the modified protein encoding sequence may have an increased rare hexamer percentage of 1%, 5%, 10%, 25%, 50%, 100%, 1,000%, 10,000%, or 100,000%.

In some embodiments, the modified protein encoding sequence may have a reduced FD bias compared to the target protein encoding sequence. The reduction is determined over the length of the protein encoding sequence, and is at least about 0.05, or at least about 0.1, or at least about 0.15, or at least about 0.2, or at least about 0.3, or at least about 0.4. If expressed as absolute FD bias, the FD bias of the modified protein encoding sequence can be about −0.05 or less, or about −0.1 or less, or about −0.15 or less, or about −0.2 or less, or about −0.3 or less, or about −0.4 or less.

In some embodiments, the modified protein encoding sequence may have a reduced FI bias compared to the target protein encoding sequence. The reduction is determined over the length of the protein encoding sequence, and is at least about 0.05, or at least about 0.1, or at least about 0.15, or at least about 0.2, or at least about 0.3, or at least about 0.4. If expressed as absolute FI bias, the FI bias of the modified protein encoding sequence can be about −0.05 or less, or about −0.1 or less, or about −0.15 or less, or about −0.2 or less, or about −0.3 or less, or about −0.4 or less.

In some embodiments, the modified protein encoding sequence may comprise only FD rare hexamers, only FI rare hexamers, or a combination of FD and FI rare hexamers.

A modified protein encoding sequence according to the present disclosure is expected to have reduced expression compared to the target protein encoding sequence. In some embodiments, the modified protein encoding sequence has reduced expression in mammalian cells compared to the target protein encoding sequence. In other embodiments, the modified protein encoding sequence has reduced expression in human cells compared to the target protein encoding sequence.

The level of attenuation of expression of the modified protein encoding sequence may be designed according to the number and type of rare hexamers in the sequence, where a greater number of rare hexamers typically leads to greater attenuation. A more attenuated sequence may be designed by, for example, inserting a greater number of rare hexamers, or inserting rarer (i.e., more depleted) hexamers into the modified protein encoding sequence. Rare FD hexamers are attenuating only in the reading frame, and should be inserted into the modified protein encoding sequence in the reading frame. Rare FI hexamers are attenuating in all frames, and therefore may be inserted in any frame.

In other embodiments, the level of attenuation may be adjusted by inserting more or less hexamers of approximately the same “rarity”, inserting fewer of the rarest hexamers, or a large number of minimally rare hexamers, according to design parameters as understood by those of ordinary skill in the art. For example, in the design of a modified viral protein for use in a vaccine, the number of rare hexamers may be greater than some minimum threshold so as to decrease the possibility of reversion to wild type.

In some embodiments, the rare hexamers chosen to attenuate expression of the modified protein encoding sequence are with respect to the organism in which the protein will be expressed rather than the organism of the target protein encoding sequence. For example, where the modified protein encoding sequence is a viral protein, one may determine the rarity of hexamers with respect to the host organism, e.g., humans, rather than a bioinformatics analysis of the genome of the virus from which the viral protein encoding sequence was derived.

In other embodiments, the rare hexamers may be inserted in one or more protein encoding sequence, or only a portion of the sequence. For example, because the 5′ region of the open reading frame is important for expression, a certain number of nucleotides at the start of the protein encoding sequence may be unchanged with reference to the target protein encoding sequence, while the rare hexamer content may be increased in other portions of the protein encoding sequence.

According to the invention, the rare hexamer content of a protein encoding sequence can be altered independently of codon usage. For example, in a protein encoding sequence of interest, the rare hexamer content can be altered simply by directed rearrangement (or shuffling) of its codons. In particular, the same codons that appear in the target sequence, which can be of varying frequency in the host organism, are used in the altered sequence, but in different positions. In the simplest form, because the same codons are used as in the target sequence, codon usage over the modified protein coding region remains unchanged (as does the encoded amino acid sequence). Nevertheless, certain codons appear in new contexts, that is, preceded by and/or followed by codons that encode the same amino acid as in the target sequence, but employing a different nucleotide triplet. Ideally, the rearrangement of codons results in an increased number of rare hexamers. In other embodiments, the rare hexamers may be introduced by substitution of synonymous codons into the target sequence and resulting in the modified protein encoding sequence.

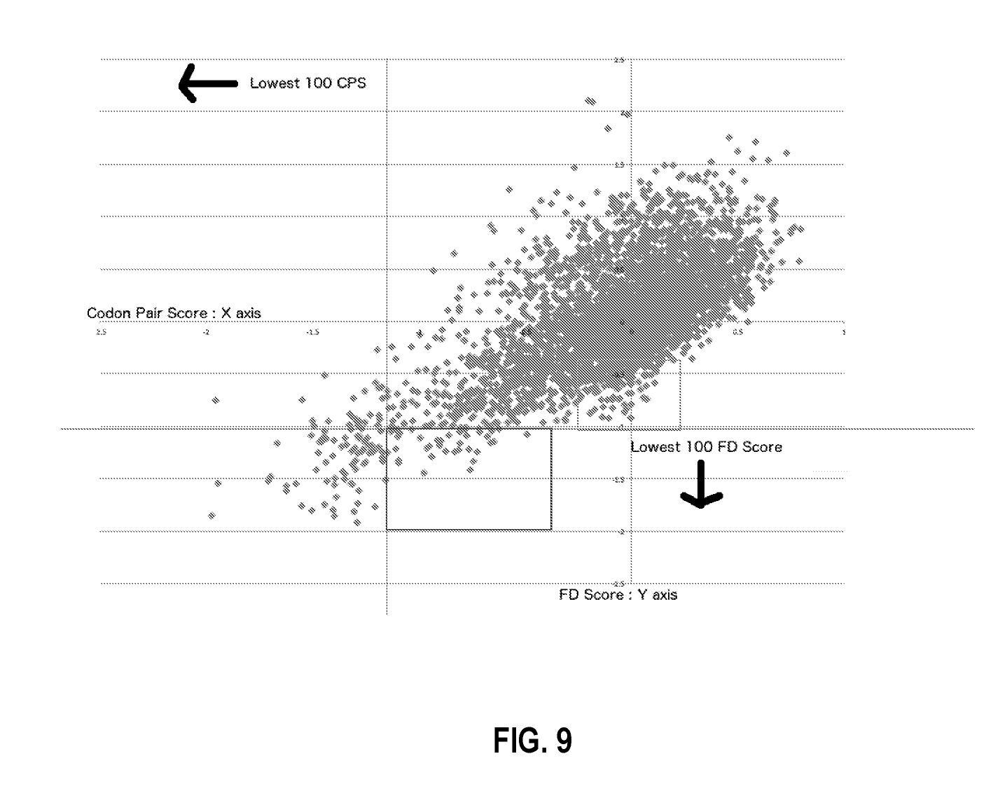

The rare hexamer content of a protein encoding sequence can also be altered independently of codon pair usage. Thus, the modified protein encoding sequence may have increased hexamer content while the codon pair bias of the modified protein encoding sequence is approximately unchanged. For example, FIG. 9 illustrates a scatterplot with H. sapiens codon pair scores on the x-axis and H. sapiens FD scores on the y axis. The bottom 100 CPS hexamers are indicated by all dots to the left of the vertical line at approximately −1.15 on the x-axis. The bottom 100 FD score hexamers are indicated by all dots lower than the horizontal line at approximately −1 on the y-axis. There are a significant number of hexamers (i.e., dots) in the lower right hand quadrant defined by these two axes (indicated by the box). These hexamers are in the lowest 100 FD scoring hexamers, but are not included in the lowest 100 CPS scoring hexamers. For any number (lowest scoring 50, 100, 150, etc.) one could draw similar axes and return similar results. Alternatively, if defining the modified protein encoding sequence according to CPB and FD bias, the box centered at 0 on the x-axis contains hexamers with low FD scores, yet the same set of hexamers have a neutral scoring CPS. Synonymous mutations including these hexamers would also create a low FD bias sequence with a neutral CPB.

Accordingly, the present disclosure also provides a method of reducing the expression of a target protein comprising introducing into the target protein encoding sequence a plurality of rare hexamers. In some embodiments, the hexamers are introduced by rearranging synonymous codons. In other embodiments, the hexamers are introduced by substituting synonymous codons.

In some embodiments, the modified protein encoding sequence may be further modified according to other parameters such codon usage, codon pair bias, RNA secondary structure and CpG dinucleotide content, C+G content, translation frameshift sites, translation pause sites, or any combination thereof.

The term “target” protein encoding sequence is used herein to refer to protein encoding sequences from which modified sequences of the present disclosure are derived. Target sequences are usually “wild type” or “naturally occurring” prototypes. However, target sequences may also include mutants specifically created or selected in the laboratory on the basis of real or perceived desirable properties. Accordingly, target sequences that are candidates for modification according to the present disclosure include mutants of wild type or naturally occurring protein encoding sequences that have deletions, insertions, amino acid substitutions and the like, and also include mutants which have codon substitutions.

The term “derived from” is used to describe that the modified protein encoding sequence is modified with respect to a target protein encoding sequence. That is, the target protein encoding sequence is used as a starting sequence to which changes are made (e.g., through either synonymous shuffling of codons or synonymous substitution of codons). By shuffling or substituting synonymous codons to increase the rare hexamer content, the modified protein encoding sequence will encode the same polypeptide sequence as that of the target protein encoding sequence from which it is derived. However, it is also contemplated that additional mutations to the modified protein encoding sequence can be made such that the resulting amino acid sequence differs from the polypeptide encoded by the target protein encoding sequence. A modified protein encoding sequence that results in a different amino acid sequence compared to the protein encoded by the target protein encoding sequence is nonetheless said to be derived from the target protein encoding sequence.

Algorithms for Sequence Design

In some embodiments, the modified protein encoding sequences may be designed using computer-based algorithms. Several novel algorithms exist for gene design that optimize the DNA sequence for particular desired properties while simultaneously coding for the given amino acid sequence. In particular, algorithms for maximizing or minimizing the desired RNA secondary structure in the sequence (Cohen and Skiena, 2003) as well as maximally adding and/or removing specified sets of patterns (Skiena, 2001), have been developed. The former issue arises in designing viable viruses, while the latter is useful to optimally insert restriction sites for technological reasons. The extent to which overlapping genes can be designed that simultaneously encode two or more genes in alternate reading frames has also been studied (Wang et al., 2006). This property of different functional polypeptides being encoded in different reading frames of a single nucleic acid is common in viruses and can be exploited for technological purposes such as weaving in antibiotic resistance genes.

The first generation of design tools for synthetic biology has been built, as described by Jayaraj et al. (2005) and Richardson et al. (2006). These focus primarily on optimizing designs for manufacturability (i.e., oligonucleotides without local secondary structures and end repeats) instead of optimizing sequences for biological activity. These first-generation tools may be viewed as analogous to the early VLSI CAD tools built around design rule-checking, instead of supporting higher-order design principles.

As exemplified herein, a computer-based algorithm can be used to manipulate the rare hexamer content of any protein encoding sequence. The algorithm may have the ability to shuffle existing codons and to evaluate the resulting rare hexamer content, and then to reshuffle the sequence, optionally locking in particularly “valuable” hexamers. Other parameters, such as the free energy of folding of RNA, may optional be under the control of the algorithm as well, in order to avoid creation of undesired secondary structures. The algorithm can be used to find a sequence with a defined number of specific rare hexamers, and in the event that such a sequence does not provide a viable protein encoding sequence, the algorithm can be adjusted to find sequences that are slightly less enriched with rare hexamers. In addition, or alternatively, the procedure may allow enrichment of the rare hexamer content by choosing a codon pair without a requirement that the codons be swapped out from elsewhere in the protein encoding sequence, i.e., the rare hexamers may be directly substituted into the target protein encoding sequence.

Quality Control Pathways and Permissive Cell Lines

This invention also provides a modified host cell line specially isolated or engineered to be permissive for a modified organism that is inviable or inefficiently produced in a wild type host cell. Since the attenuated organism cannot grow in normal (e.g., wild type) host cells, it is dependent on the specific helper cell line for growth. Various embodiments of the instant modified cell line permit the growth of a modified virus, wherein the genome of said cell line has been altered according to the type of hexamer, (i.e., rare FD or rare FI hexamers) with which the organism has been modified. In one embodiment, the modified cell line may have degraded translation quality control pathways to permit the growth of an organism modified that contains an increased number of rare FD hexamers compared to the unmodified organism.

In one embodiment, a modified host cell line is specially isolated or engineered to be permissive for a modified virus that is inviable in a wild type host cell. This provides a very high level of safety for the generation of virus for vaccine production. Various embodiments of the instant modified cell line permit the growth of a modified virus, wherein the genome of said cell line has been altered according to the type of hexamer, (i.e., rare FD or rare FI hexamers) with which the virus has been modified.

Attenuation by FD or FI rare hexamers cause attenuation by provoking the degradation of the messenger RNA by so-called “quality control” pathways. These quality control pathways include, but are not limited to, the UPF1 pathway, the Dom34 pathway, and the Rqcl pathway, or equivalent mammalian mRNA quality control pathways UPF1, Pelota, and Tcf25. These various pathways are involved in degrading mRNAs with specific kinds of defects, such as the defects caused by rare hexamers. Importantly, cells lacking one of the quality control pathways can survive, but now are defective in mRNA degradation of mRNAs with the specific defects.

Thus, to make a permissive cell line for an attenuated organism, the organism is attenuated using just one type of rare hexamer, such as FD hexamers. Correspondingly, a cell line is generated, such as an UPF1 mutant cell line, that fails to recognize the particular mRNA defect. The attenuated organism can now more efficiently reproduce in this cell line, whereas it could not efficiently reproduce in a the cell line without the permissive modification(s).

Because the quality control pathways are normally devoted to resolving problems with aberrant translation, manipulations that cause aberrant translation provoke a response from the quality control pathways. The component proteins of the pathways are limited in amount, and can be titrated out (i.e., the pathway can be saturated). Thus, application of an aminoglycoside antibiotic such as G418 can titrate out (saturate) the quality control pathways, allowing stability of mRNAs containing defects such as engineered rare hexamers. Thus, instead of making a mutant cell line, one can also grow a wild-type or otherwise nonpermissive cell line under conditions, such as aminoglycoside antibiotics, that effectively inactivate quality control pathways by saturating them with other defects.

Large Scale DNA Assembly

In recent years, the decreasing costs and increasing quality of oligonucleotide synthesis have made it practical to assemble large segments of DNA (at least up to about 10 kb) from synthetic oligonucleotides. Commercial vendors such as Blue Heron Biotechnology, Inc. (Bothwell, Wash.) (and also others) currently synthesize, assemble, clone, sequence-verify, and deliver a large segment of synthetic DNA of known sequence for the price of about $1.50 per base. Thus, purchase of synthesized viral genomes from commercial suppliers is a convenient and cost-effective option. Furthermore, new methods of synthesizing and assembling very large DNA molecules at low costs are emerging (Tian et al., 2004). The Church lab has pioneered a method that uses parallel synthesis of thousands of oligonucleotides (for instance, on photo-programmable microfluidics chips, or on microarrays available from Nimblegen Systems, Inc., Madison, Wis., or Agilent Technologies, Inc., Santa Clara, Calif.), followed by error reduction and assembly by overlap PCR. These methods have the potential to reduce the cost of synthetic large DNAs to less than 1 cent per base. The improved efficiency and accuracy, and rapidly declining cost, of large-scale DNA synthesis provides an impetus for the development and broad application of modifying protein encoding sequences by altering their rare hexamer content.

Vaccine Compositions

In some embodiments, the modified protein encoding sequence may be a viral protein. For example, the influenza virus has eight separate genomic segments encoding Polymerase PB2, Polymerase PB1, Polymerase PA, hemagglutinin HA, nucleoprotein NP, neuraminidase NA, matrix proteins M1 and M2, and nonstructural protein NS1. One or more of these genomic segments, such as HA and/or NA, may be modified according to the present disclosure to generate a modified virus. In another non-limiting example, poliovirus is a small non-enveloped virus with a single stranded (+) sense RNA genome of 7.5 kb in length. Upon cell entry, the genomic RNA serves as an mRNA encoding a single polyprotein that after a cascade of autocatalytic cleavage events gives rise to full complement of functional poliovirus proteins. The same genomic RNA serves as a template for the synthesis of (−) sense RNA, an intermediary for the synthesis of new (+) strands that either serve as mRNA, replication template or genomic RNA destined for encapsidation into progeny virions. A modified PV sequence may be designed according to the present disclosure by increasing the rare hexamer content over the entire PV sequence, or a portion of the sequence. The expression of the modified viral proteins will be reduced, and the virus attenuated. These attenuated viruses may be useful in vaccine compositions and for inducing protective immune responses, as disclosed in WO 2008/121992, WO 2011/044561, WO 2014/145290, and WO 2016/037187, all of which are incorporated herein in its entirety.

Viral attenuation and induction of protective immune responses can be confirmed in ways that are well known to one of ordinary skill in the art, including, but not limited to, methods and assays such as plaque assays, growth measurements, reduced lethality in test animals, and protection against subsequent infection with a wild type virus.

The present invention also provides a vaccine composition for inducing a protective immune response in a subject comprising any of the modified viruses described herein and a pharmaceutically acceptable carrier.

It should be understood that a modified virus of the invention, where used to elicit a protective immune response in a subject or to prevent a subject from becoming afflicted with a virus-associated disease, is administered to the subject in the form of a composition additionally comprising a pharmaceutically acceptable carrier. Pharmaceutically acceptable carriers are well known to those skilled in the art and include, but are not limited to, one or more of 0.01-0.1M and preferably 0.05M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate and magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Pharmaceutically acceptable carriers can further comprise minor amounts of auxiliary substances such as wetting or emulsifying agents, preservatives and other additives, such as, for example, antimicrobials, antioxidants and chelating agents, which enhance the shelf life and/or effectiveness of the active ingredients. The instant compositions can, as is well known in the art, be formulated so as to provide quick, sustained or delayed release of the active ingredient after administration to a subject.

In various embodiments of the instant vaccine composition, the modified virus (i) does not substantially alter the synthesis and processing of viral proteins in an infected cell; (ii) produces similar amounts of virions per infected cell as wt virus; and/or (iii) exhibits substantially lower virion-specific infectivity than wt virus. In further embodiments, the attenuated virus induces a substantially similar immune response in a host animal as the corresponding wt virus.

In addition, the present invention provides a method for eliciting a protective immune response in a subject comprising administering to the subject a prophylactically or therapeutically effective dose of any of the vaccine compositions described herein. This invention also provides a method for preventing a subject from becoming afflicted with a virus-associated disease comprising administering to the subject a prophylactically effective dose of any of the instant vaccine compositions. In embodiments of the above methods, the subject has been exposed to a pathogenic virus. “Exposed” to a pathogenic virus means contact with the virus such that infection could result.

The invention further provides a method for delaying the onset, or slowing the rate of progression, of a virus-associated disease in a virus-infected subject comprising administering to the subject a therapeutically effective dose of any of the instant vaccine compositions.

As used herein, “administering” means delivering using any of the various methods and delivery systems known to those skilled in the art. Administering can be performed, for example, intraperitoneally, intracerebrally, intravenously, orally, transmucosally, subcutaneously, transdermally, intradermally, intramuscularly, topically, parenterally, via implant, intrathecally, intralymphatically, intralesionally, pericardially, or epidurally. An agent or composition may also be administered in an aerosol, such as for pulmonary and/or intranasal delivery. Administering may be performed, for example, once, a plurality of times, and/or over one or more extended periods.

Eliciting a protective immune response in a subject can be accomplished, for example, by administering a primary dose of a vaccine to a subject, followed after a suitable period of time by one or more subsequent administrations of the vaccine. A suitable period of time between administrations of the vaccine may readily be determined by one skilled in the art, and is usually on the order of several weeks to months. The present invention is not limited, however, to any particular method, route or frequency of administration.

A “subject” means any animal or artificially modified animal. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems, and CD155tg transgenic mice expressing the human poliovirus receptor CD155. In a preferred embodiment, the subject is a human. Preferred embodiments of birds are domesticated poultry species, including, but not limited to, chickens, turkeys, ducks, and geese.

A “prophylactically effective dose” is any amount of a vaccine that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood of the disorder's onset in the subject, by at least two-fold, preferably at least ten-fold. For example, if a subject has a 1% chance of becoming infected with a virus, a two-fold reduction in the likelihood of the subject becoming infected with the virus would result in the subject having a 0.5% chance of becoming infected with the virus. Most preferably, a “prophylactically effective dose” induces in the subject an immune response that completely prevents the subject from becoming infected by the virus or prevents the onset of the disorder in the subject entirely.

As used herein, a “therapeutically effective dose” is any amount of a vaccine that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

Certain embodiments of any of the instant immunization and therapeutic methods further comprise administering to the subject at least one adjuvant. An “adjuvant” shall mean any agent suitable for enhancing the immunogenicity of an antigen and boosting an immune response in a subject. Numerous adjuvants, including particulate adjuvants, suitable for use with both protein- and nucleic acid-based vaccines, and methods of combining adjuvants with antigens, are well known to those skilled in the art. Suitable adjuvants for nucleic acid based vaccines include, but are not limited to, Quil A, imiquimod, resiquimod, and interleukin-12 delivered in purified protein or nucleic acid form. Adjuvants suitable for use with protein immunization include, but are not limited to, alum, Freund's incomplete adjuvant (FIA), saponin, Quil A, and QS-21.

The invention also provides a kit for immunization of a subject with an attenuated virus of the invention. The kit comprises the attenuated virus, a pharmaceutically acceptable carrier, an applicator, and instructional material for the use thereof. In further embodiments, the attenuated virus may be one or more poliovirus, one or more rhinovirus, one or more influenza virus, etc. More than one virus may be preferred where it is desirable to immunize a host against a number of different isolates of a particular virus. The invention includes other embodiments of kits that are known to those skilled in the art. The instructions can provide any information that is useful for directing the administration of the attenuated viruses.

Of course, it is to be understood and expected that variations in the principles of the invention herein disclosed can be made by one skilled in the art and it is intended that such modifications are to be included within the scope of the present invention. The following Examples further illustrate the invention, but should not be construed to limit the scope of the invention in any way. Detailed descriptions of conventional methods, such as those employed in the construction of recombinant plasmids, transfection of host cells with viral constructs, polymerase chain reaction (PCR), and immunological techniques can be obtained from numerous publications, including Sambrook et al. (1989) and Coligan et al. (1994). All references mentioned herein are incorporated in their entirety by reference into this application.

Full details for the various publications cited throughout this application are provided at the end of the specification immediately preceding the claims. The disclosures of these publications are hereby incorporated in their entireties by reference into this application. However, the citation of a reference herein should not be construed as an acknowledgement that such reference is prior art to the present invention.

### EXAMPLES

### Example 1

Gene Attenuation Using Codon Pair Bias

To study the mechanism of attenuation by depleted codon pairs, modified genomes of the yeast S. cerevisiae were studied. Attenuation by codon pair deoptimization has not previously been demonstrated in any cellular eukaryote. The two amino-acid biosynthetic genes, HIS3 (220 codons) and LYS2 (1392 codons), for the synthesis of histidine and lysine, respectively, were used.

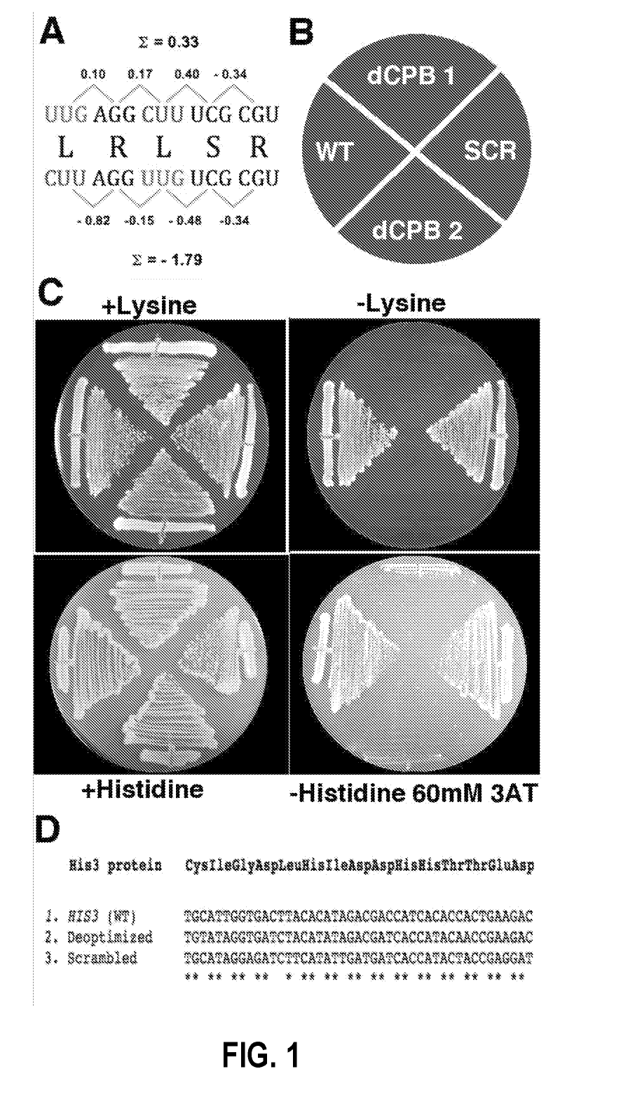

A codon shuffling heuristic approach was used to design genes containing depleted codon pairs (Coleman et al., 2008). The software repeatedly “shuffles” the positions of existing synonymous codons within a gene, aiming for shuffles that generate depleted codon pairs. For example, shuffling Leu UUG with Leu CUU as shown in FIG. 1A creates four new codon pairs. Because only codons existing in the wild-type gene are used, this procedure does not change the amino acid sequence of the gene, nor does it change the frequency of any of the codons used in the gene. That is, the shuffled genes are the same as the wild-type genes in amino acid sequence and in codon usage. The deoptimized genes are denoted herein with a “d” prefix (e.g., dHIS3). Because the 5′ region of the open reading frame may be important for expression, the first 60 nucleotides (for HIS3) or 120 nucleotides (LYS2) after the start codon were left unchanged. WT HIS3 (SEQ ID NO: 1) has a codon pair score of 6; while the deoptimized genes have scores around −50. WT LYS2 (SEQ ID NO: 11) has a codon pair score of 39; while the deoptimized LYS2 genes have scores around −250.

Because altering the natural sequence could be deleterious for various unknown reasons, a “scramble” control genes were also designed, in which the software equally shuffles synonymous codons, but without selecting for any particular codon pair arrangements. This results in a synthetic “scramble” gene with the same amino acid sequence, codon usage, and codon pair score as wild-type. However, it also has about the same number of silent mutations as the codon pair deoptimized gene. Thus, as a control for effects of nucleotide rearrangement, a gene with shuffled synonymous codons and a low codon pair score (the codon pair deoptimized gene) was compared against an equally shuffled gene with a wild-type codon pair score (the scramble control gene). This comparison shows that it is specifically the low codon pair score that is responsible for any observed changes in gene function.

The codon pair deoptimized genes were strikingly attenuated, while the scramble controls were not (FIGS. 1B, C). The first two deoptimized versions of LYS2, dLYS2-1 and dLYS2-2, were genetically completely non-functional (i.e. Lys-) (FIGS. 1B, C). Two deoptimized versions of HIS3, a much shorter gene, with less negative codon pair scores, remained functional (i.e., His+). However, challenge with 3-aminotriazole, an inhibitor of the His3 enzyme, showed that both deoptimized genes were attenuated (FIGS. 1B, C). Many other codon pair deoptimized versions of LYS2 and HIS3 were made, and all of them are attenuated. The scramble controls remained Lys+, or His+, respectively, showing that the low codon pair score, and not the codon pair rearrangement as such, is responsible for attenuation.

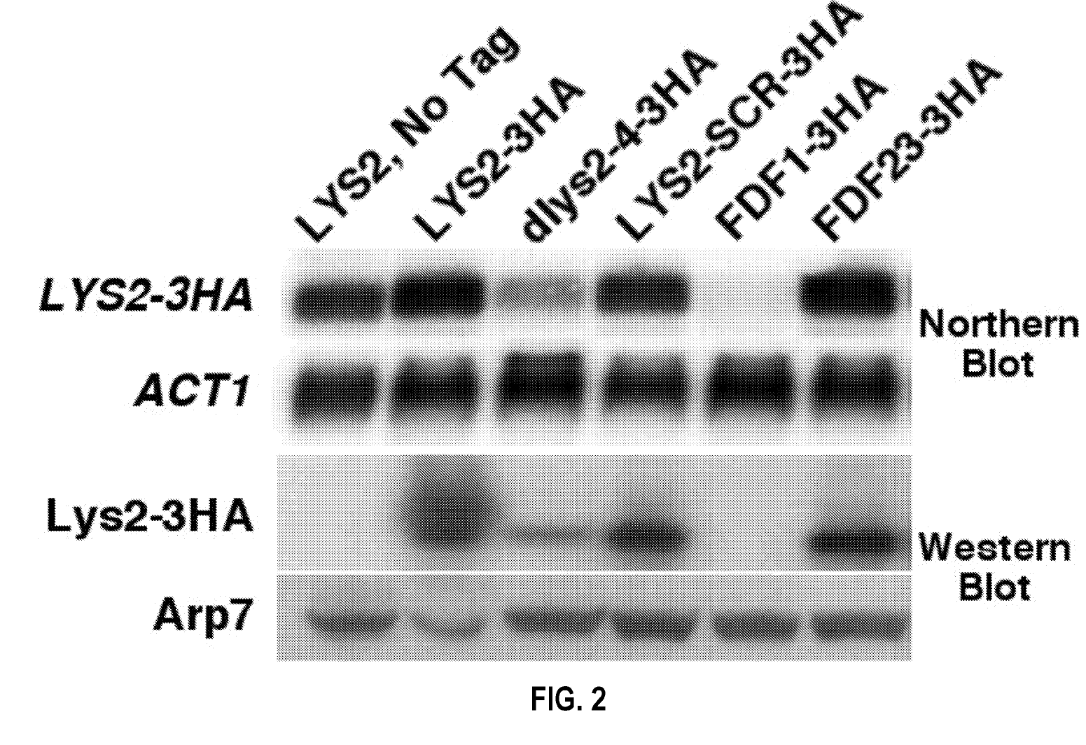

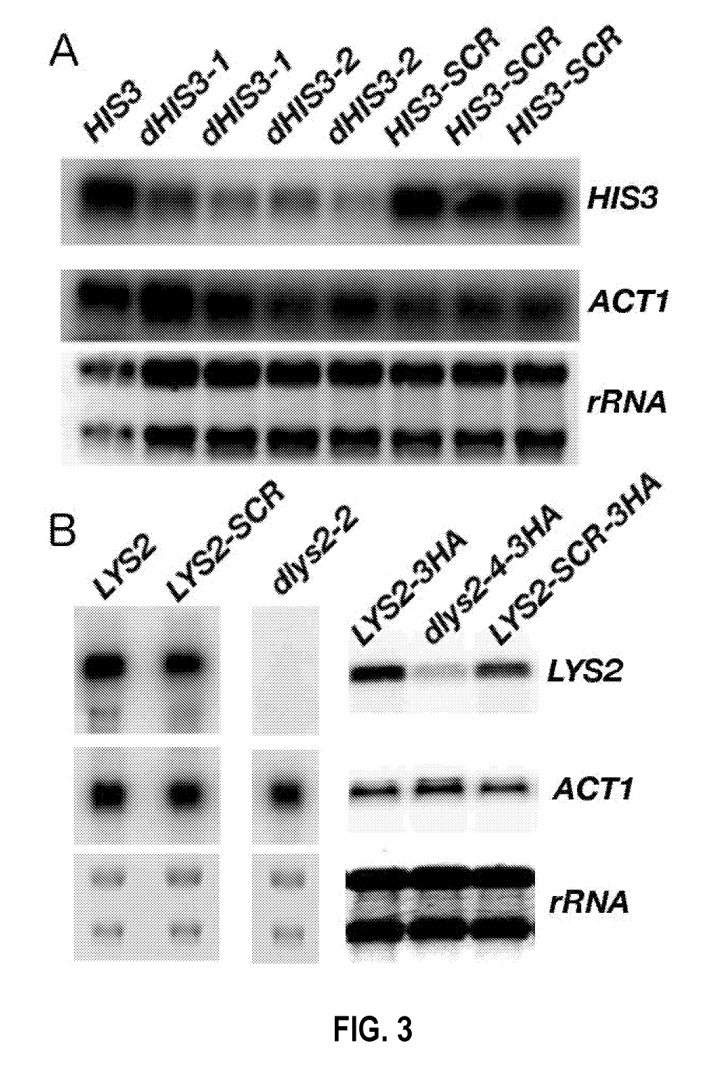

Western analysis showed that dLYS2 and dHIS3 alleles produced greatly reduced levels of protein, as expected from the reduced function. But both Northern analysis and RNA-Seq showed that dLYS2 and dHIS3 mRNA levels were also much lower than wild-type or scramble controls. Western and Northern analysis of wild-type, scrambled (SCR) and codon-pair deoptimized alleles of LYS2 is shown in FIG. 2. Northern analysis comparing mRNA levels of WT HIS3, two biological replicates each of the CPB deoptimized alleles dHIS3-1 (SEQ ID NO: 3) and dHIS3-2 (SEQ ID NO: 4), and three biological replicates of the scramble HIS3 allele (HIS3-scr; SEQ ID NO: 2) is shown in FIG. 3A, and Northern analysis comparing mRNA levels of WT LYS2 (either tagged with the HA epitope or not), scrambled LYS2 (LYS2-scr; SEQ ID NO: 12) (tagged with the HA epitope or not) with two CPB deoptimized alleles, dlys2-2 (SEQ ID NO: 13), and dlys2-4-3HA (SEQ ID NO: 14) is shown in FIG. 3B. Loading controls in FIG. 3A and 3B are the ACT1 mRNA, and two ribosomal RNAs.

In general, the decrease in protein was well-correlated with the decrease in mRNA. Thus the effect of codon-pair deoptimization is seen at the mRNA level; presumably the low levels of mRNA are causing the low levels of protein and low levels of genetic function.

### Example 2

Identification of Frame Dependent (FD) and Frame Independent (FI) Hexamers

To examine whether the attenuation was connected to defects in translation, the question of whether the effects of codon pairs were specific to the reading frame was examined Here, it was reasoned that if some hexamer XXXXXX corresponding to a rare codon pair were directly destabilizing mRNA, it would do so in any frame (i.e., XXX XXX, nXX XXX Xnn, and nnX XXX XXn would be equally destabilizing). In contrast, if a hexamer were working through translation, it would be destabilizing only in the reading frame (i.e., destabilizing as XXX XXX, but not as nXX XXX Xnn or nnX XXX XXn, which usually specify different codons and tRNAs). Therefore, the codon pair score was adapted to investigate the enrichment/depletion of hexamers in each possible frame.

Frame Dependent and Frame Independent scores were calculated by equation 1 and 2 respectively:

$$\begin{matrix}FDscore ( hexamer ) = {CPS}_{Frame 1} ( hexamer ) - \frac{{CPS}_{Frame 2} ( hexamer ) + {CPS}_{Frame 3} ( hexamer )}{2} & eq . ( 1 ) \\ FIscore ( hexamer ) = \frac{\begin{matrix}{CPS}_{Frame 3} ( hexamer ) + \\ {CPS}_{Frame 2} ( hexamer ) + {CPS}_{Frame 3} ( hexamer )\end{matrix}}{3} & eq . ( 2 )\end{matrix}$$

Frame Dependent scores for hexamers containing an out-of-frame stop codon were altered as in equation 3, to allow for the fact that such hexamers are not permissible in one of the three frames. Hexamers containing out-of-frame stop codons were excluded from Frame Independent score calculation, as they are inherently Frame Dependent.

FDscore(OOFS hexamer)=CPS_{Frame 1}(OOFS hexamer)−CPS_{Frame 2 or 3} (OOFS hex er) eq.(3)

It was then examined whether depleted hexamers were depleted (a) equally in all three frames; or (b) only in the reading frame. Surprisingly, the hexamers defined by depleted codon pairs fell into both classes in similar numbers. The first class, depleted equally in all three frames, was called “Frame Independent” hexamers, or “FI.” These are candidates for “rare hexamers”, sequences which potentially affect the mRNA from any reading frame, presumably independently of translation. The second class, depleted only in the reading frame, was called “Frame Dependent” hexamers, or “FD”. These are candidates for codon pairs that presumably somehow affect translation (and so are dependent on the reading frame in which they occur).

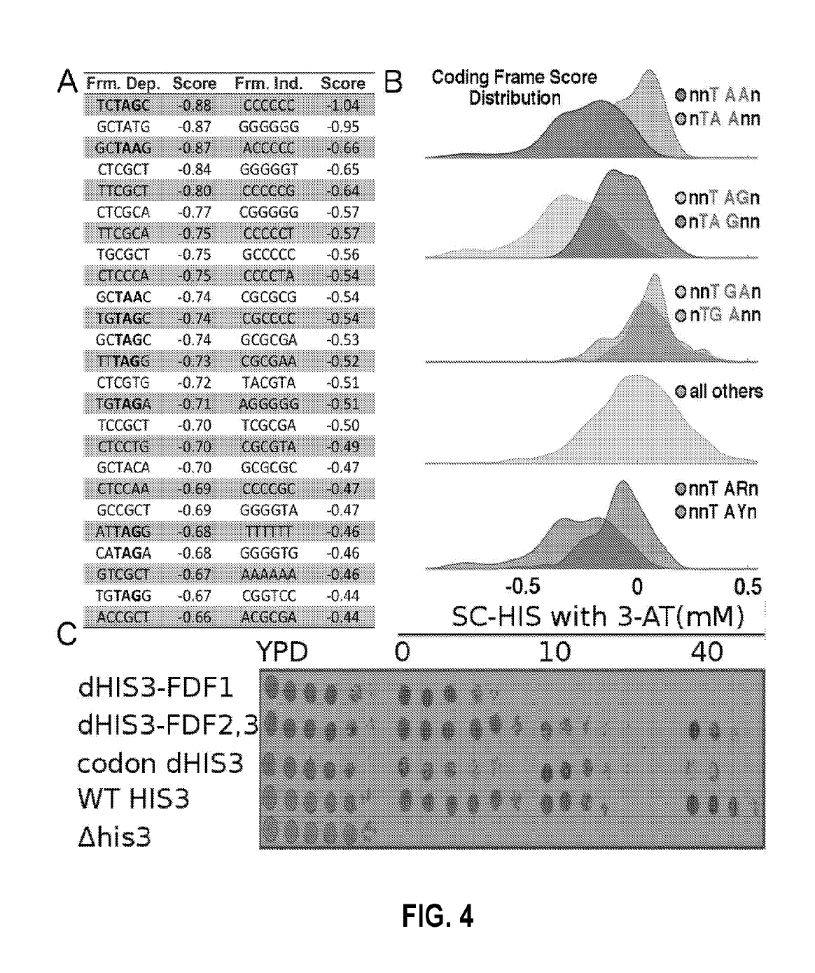

The sequences of the yeast FD and FI hexamers were diverse. Several other species were examined, and all these other species likewise had both Frame-Dependent and Frame-Independent hexamers, and common features emerged. FIG. 4A shows the 25 most-depleted (most negative scoring) Frame Dependent and Frame Independent hexamers after averaging scores over eight organisms, S. cerevisiae, S. pombe, E. coli, C. elegans, D. rerio, D. melanogaster, A. thaliana, and H. sapiens, and the full set of FD and FI scores for all hexamers is provided herewith a Supplemental Tables S2 and S3, respectively.

The depleted Frame Independent (FI) hexamers contained three types of sequences: GC-rich sequences, homopolymers, and, to some extent, palindromes.

The depleted Frame Dependent (FD) hexamers contained mainly two types of sequences, those with a central “CG” (10 of the worst 25), and those with an out-of-frame TAA or TAG stop codon in the −1 reading frame (10 of the worst 25). The latter are called “OOFS”, for “Out-Of-Frame-Stops”. Further analysis showed that in yeast, essentially every codon pair that generates a TAA or TAG in the -1 frame is a depleted codon pair (FIG. 4B). By contrast, TAA and TAG in the −2 frame were not depleted, nor was TGA in either the −1 or −2 frame (FIG. 4B), nor was TAT or TAC in any frame (FIG. 4B).

### Example 3

Attenuation by Rare FD and FI Hexamers

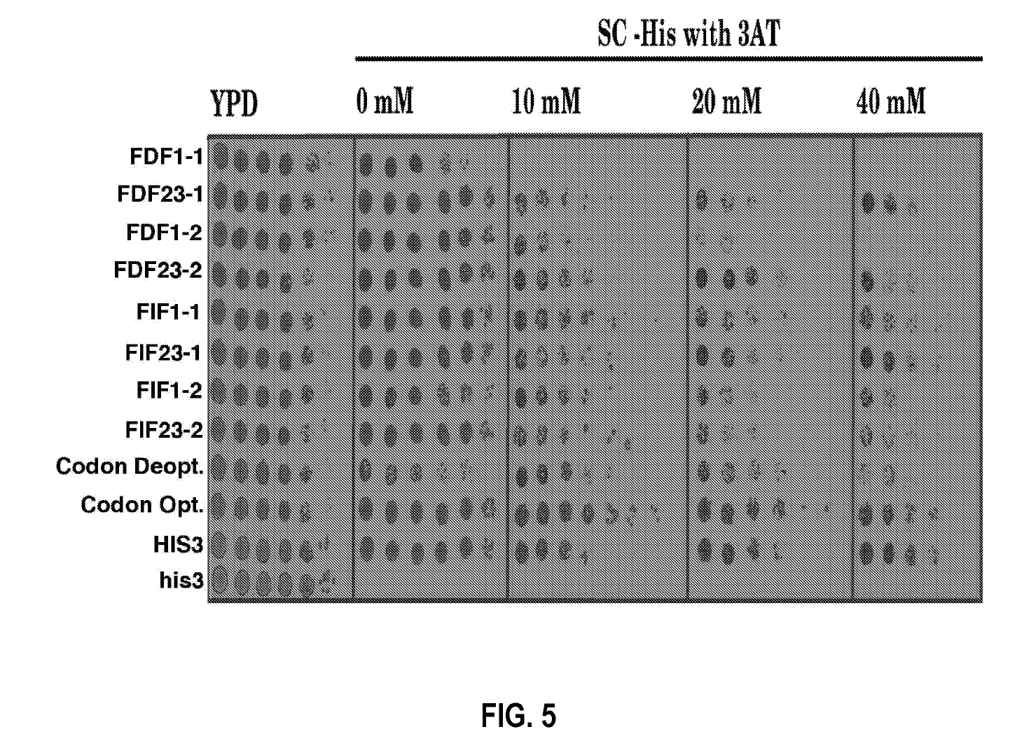

To investigate whether the two newly-identified classes of depleted codon pairs were functionally significant, new classes of deoptimized LYS2 and HIS3 genes were built to test the function of the Frame Independent and Frame Dependent hexamers. First, genes were deoptimized using only yeast Frame Independent hexamers. One gene design was enriched with yeast FI hexamers only in the reading frame, while a second design was enriched with yeast FI hexamers only in the −1 and −2 frames. One LYS2 and two HIS3 alleles each were synthesized. As predicted, the FI hexamers were moderately and equally attenuating regardless of which frame they are in (FIG. 5). This confirms that these hexamers are (a) attenuating; and (b) reading frame-independent, and therefore probably not working via particular codons, and possibly not working via translation.

Second, genes were deoptimized using only yeast Frame Dependent (FD) hexamers. One gene design was enriched with yeast FD hexamers only in the reading frame, while a second design was enriched with yeast FD hexamers only in the -1 and -2 frames. The FD hexamers are very strongly attenuating in the reading frame, but not significantly attenuating in the two other frames. FIG. 5 shows a growth of serial dilutions of attenuated alleles of HIS3 on SC-His with 3-aminotriazole. FDF1-1 (HIS3-SEQ ID NO: 5; LYS2-SEQ ID NO: 15) and FDF1-2 (HIS3-SEQ ID NO: 7; LYS2-SEQ ID NO: 16) are attenuated with “Frame Dependent” hexamers in frame 1 (where those hexamers are naturally depleted), while FDF23-1 and FDF23-2 are control genes with “Frame Dependent” hexamers in frames 2 and 3 (where they are not naturally depleted). FIF1-1 (HIS3-SEQ ID NO: 8; LYS2-SEQ ID NO: 17) and FIF1-2 are attenuated with “Frame Independent” hexamers in frame 1 (where those hexamers are naturally depleted), while FIF23-1 (HIS3-SEQ ID NO: 9; LYS2-SEQ ID NO: 18) and FIF23-2 have the Frame Independent hexamers in frames 2 and 3 (where, these being frame-independent hexamers, they are also naturally depleted). “Codon Deopt” is an allele of HIS3 with the worst possible codon usage, while “Codon Opt” has the best possible codon usage. HIS3 and his3 are wild-type and deletion controls, respectively.

This confirms that these hexamers are (a) attenuating; and (b) dependent on reading frame, and therefore probably are working as pairs of codons, possibly at the level of translation. In general, it appeared that the Frame Dependent hexamers attenuated more strongly that the Frame Independent hexamers.

To compare the magnitude of rare hexamer attenuation to the magnitude of attenuation by the much better known “codon usage” bias, a HIS3 gene with the worst possible codon usage (i.e., using only rare codons) was synthesized (SEQ ID NO: 10). It was found that codon pair deoptimized versions of HIS3 were much more strongly attenuated than the “codon usage” allele. That is, rare hexamer attenuation gives stronger effects than codon usage.

The amount of the 100 most-depleted S. cerevisiae FD hexamers in each of the constructs, as well as the FD bias score, is shown in Table 5.

TABLE 5

| | Number of | |
|---|---|---|
| | 100 most rare | |
| | FD hexamers | FD bias score |
| WT His3 | 3 | 0.024957534 |
| HIS3scrambleallele1 | 3 | 0.043894922 |
| dHis3allele1 | 27 | −0.1651801 |
| dHis3allele2 | 19 | −0.135173878 |
| His3FDF1allele1 | 26 | −0.127564706 |
| FDF1His3allele5 | 14 | −0.088045173 |
| His3FDF23allele1 | 1 | 0.053859932 |
| His3FIF1allele1 | 5 | 0.005050255 |
| His3FIF23allele1 | 12 | −0.036471864 |
| His3CodonBiasDeoptimizedallele1 | 6 | 0.067177896 |
| WT Lys2 | 15 | 0.017281335 |
| LYS2scramble | 27 | 0.037977737 |
| dLYS2-2 | 98 | −0.132490676 |
| dLys2-4 | 93 | −0.126162732 |
| Lys2FDF1 | 79 | −0.075682933 |
| Lys2FDF23 | 7 | 0.021624991 |
| Lys2FIF1 | 33 | −0.011277512 |
| Lys2FIF23 | 33 | −0.009669779 |

### Example 4

Translation of Rare Hexamer Deoptimized Genes

The idea of translational defect was supported by two additional experiments. The translation of these codon pair deoptimized genes was directly examined Two approaches were used. First, ribosome profiling experiments, which counts the number of ribosomes associated with a particular mRNA, which can be a proxy for the rate of translation, were conducted. A diploid strain which contained one copy of wild-type HIS3, and one copy of the rare hexamer deoptimized allele dHIS3-FDF1-5 (SEQ ID NO: 6; dHIS3-FDF1-5 is a moderately attenuated allele. A strongly attenuated allele could not be used because such strains contain too little mRNA for ribosome profiling analysis.) was constructed. This heterozygous diploid strain was grown under conditions that induce HIS3 gene expression, and ribosome profiling was done on a single extract from this strain (i.e., the ribosome profiles of HIS3 and dHIS3-FDF1-5 were obtained simultaneously, in a single extract from a single culture of a single strain). Because the sequences of the WT and dHIS3-FDF1-5 alleles are very different in the deoptimized region, almost all ribosome footprints from each mRNA could be unambiguously identified and assigned to either the WT or the deoptimized gene.

RNA-Seq was also done on the same extract to quantify each mRNA. The ratio of the number of ribosome footprints for each mRNA to the number of RNA-Seq reads for each RNA was obtained. (This ratio is often called the “Translational Efficiency”, but more properly it is a ribosome density.) This ribosome density was 0.06 higher for dHIS3-FDF1-5 than for wild-type HIS3. Since both mRNAs are expressed from the same promoter in the same genomic location with the same 5′ UTR with the same 60 nucleotides at the 5′ end of the coding region, the rate of translational initiation is likely the same for both mRNAs. Thus, the higher ribosome density for dHIS3-FDF1-5 is interpreted to mean that the ribosomes are moving about 35% slower on the deoptimized mRNA than on the wild-type mRNA.

As a second approach, the number of ribosomes on WT HIS3 and dHIS3-FDF1-5 mRNAs was counted using polysome gradients. Again, the heterozygous diploid strain carrying both alleles of HIS3 was used, a polysome extract made, and this extract was run on a single sucrose gradient. This gradient was fractionated and fractions analyzed by qRT-PCR. Again, because of the large sequence difference between WT HIS3 and dHIS3-FDF1-5, the two mRNAs were easily distinguished. As shown in Fig. XX, on average, the WT HIS3 mRNA carried three ribosomes, while the average dHIS3-FDF1-5 mRNA carried four ribosomes. Again, this reflects a difference in elongation rate, implying that ribosomes move about xx % more slowly on the deoptimized mRNA. Very similar results were obtained in several repeats of this experiment.

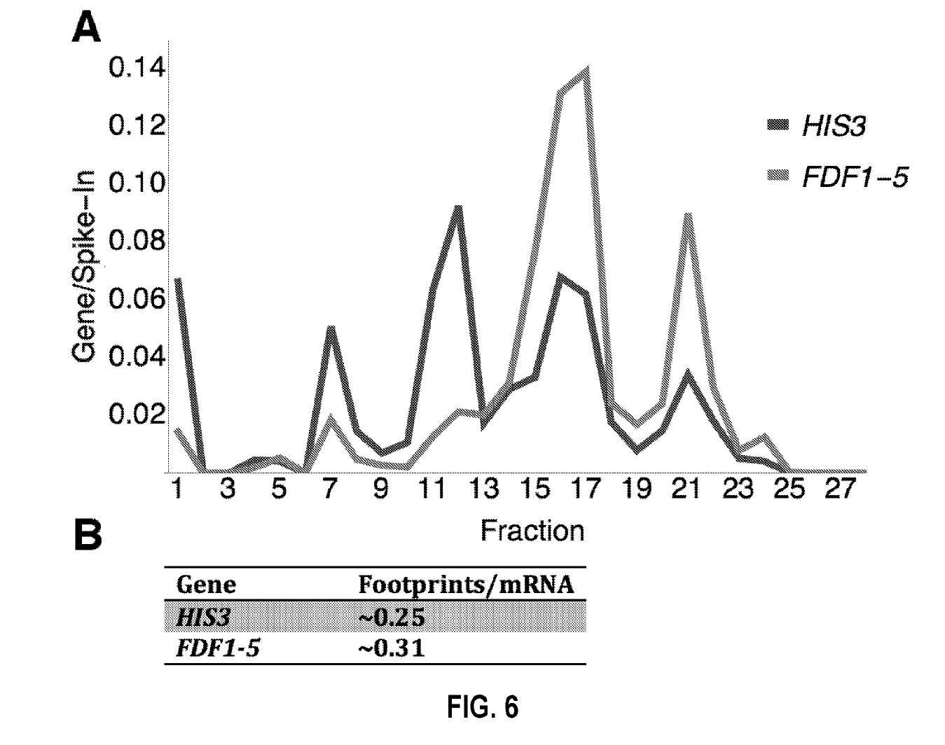

The results of these experiments are shown in FIGS. 6A and 6B.

Thus, the two approaches agreed that ribosome density is higher on the deoptimized mRNA, implying that translation is occurring more slowly. This indeed suggests that rare hexamer deoptimization is directly causing some translational defect. On the other hand, the slow-down is only about 35%. This is quite a modest effect, which seems much too small to explain the very strong phenotypic effects. Indeed, since translation is typically limited by the rate of initiation, it is not clear that slowing down elongation by 35% would necessarily have any phenotypic effect.

### Example 5

Quality Control Pathways

If Frame Dependent hexamers were causing a translational defect, then that defect might induce translation quality-control surveillance pathways to destroy the “defective” mRNA. A given molecule of mRNA can be translated hundreds of times, giving quality control pathways many chances to respond to a minor defect, and the destruction of a molecule of mRNA is irreversible. Therefore, a low-level translational defect could cause a large loss of mRNA by inducing mRNA degradation via quality control. If this hypothesis were correct, then the loss of mRNA, and much of the attenuation, would be suppressed by mutations in appropriate quality-control pathways.

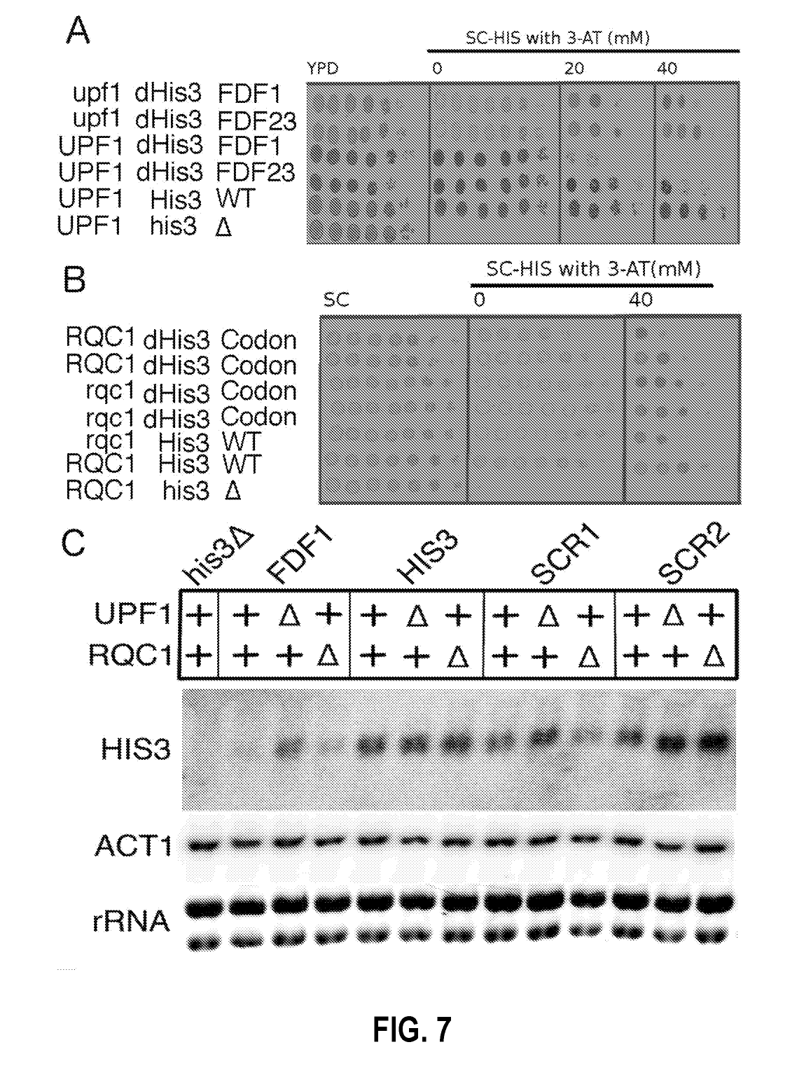

To test this idea, the quality-control genes UPF1 (nonsense-mediated decay), DOM34 (no-go decay), SKI7, and RQC1 (ribosome quality control) were mutated in strains bearing various attenuated genes in which the rare hexamer content was increased. These and other quality control pathways are highly conserved across species, including humans Strikingly, for the Frame Dependent deoptimizations, the upf1 and rqc1 mutations suppressed the functional defects to varying extents (FIG. 7A, compare row 1 with row 3; and data not shown). However, they did not suppress the defects of the Frame Independent deoptimizations, and neither dom34 nor skip showed detectable suppression of any allele (data not shown). This functional suppression was mirrored at the mRNA level (FIG. 7C), where the upfl mutation restored the level of the dHIS3-FDF1-1 mRNA to nearly WT levels. These results demonstrate that a major factor in the attenuation of FD codon-pair deoptimized genes is that translational quality-control pathways are degrading deoptimized mRNAs, presumably as a result of some still undefined defect in translation.

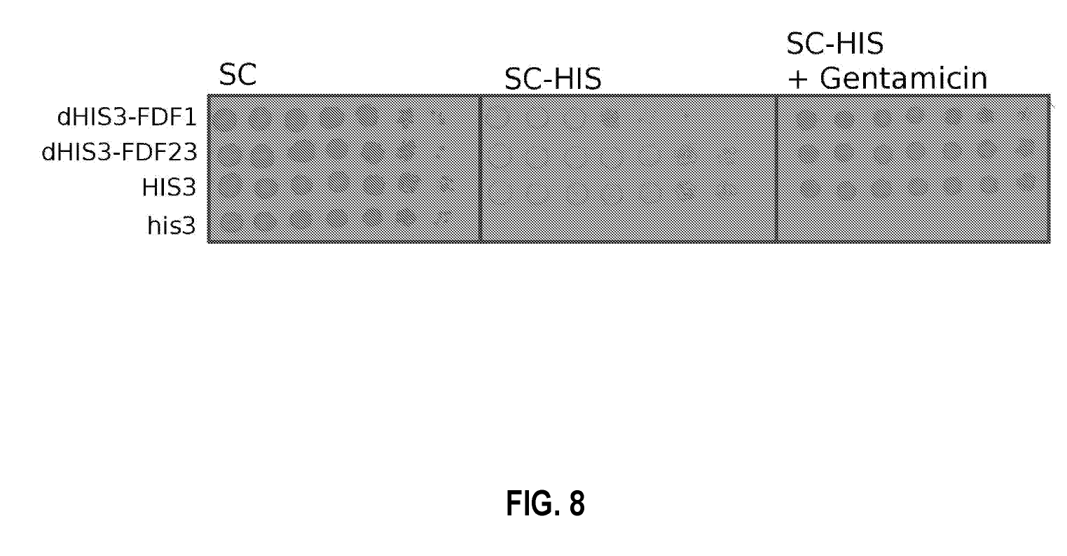

Brandman et al. observed that the components of the quality control pathways are rare, and can be titrated out by moderately severe translational stress. Therefore, as another test of the idea that quality control pathways are important for the phenotype of rare hexamer attenuated genes, strains were grown with rare hexamer deoptimized genes in the presence of small amounts of the antibiotic gentamicin, which causes translational stress. Indeed, exactly consistent with the results of Brandman et al., low dose gentamicin was also able to suppress rare hexamer attenuation (FIG. 8). An interpretation of this result is that in the presence of traces of gentamicin, translational problems are widespread, and the quality control machinery is titrated out, and so individual problematic mRNAs such as dHIS3-FDF1 escape degradation by quality control.

The results are reminiscent of those of Presnyak et al. for genes with poor codon bias. Genes deoptimized either with rare hexamers or rare codons show modest translational defects, but strikingly low levels of mRNA. Presnyak et al. found that the quality-control mutant upfl did not affect mRNA levels for codon-deoptimized HIS3. However, Presnyak et al. did not test rqc1 Rqc1 was tested on codon-deoptimized HIS3, and found that rqcl does suppress its attenuation (FIG. 7B), just as it suppresses rare hexamer deoptimized HIS3. The suppression is weak, but the attenuation is weak to begin with. Therefore, it appears that rare hexamer deoptimization and codon usage deoptimization each induce their own particular types of translational defects. These defects provoke one or more of the quality-control surveillance mechanisms to destroy the mRNA in question, and it is this quality-control mediated destruction that is, at least in part, the direct reason for the loss of mRNA and the attenuation.

### Example 6

Modified Viruses with Increased Rare Hexamer Content

Poliovirus, a member of the Picornavirus family, is a small non-enveloped virus with a single stranded (+) sense RNA genome of 7.5 kb in length. Upon cell entry, the genomic RNA serves as an mRNA encoding a single polyprotein that after a cascade of autocatalytic cleavage events gives rise to full complement of functional poliovirus proteins. The same genomic RNA serves as a template for the synthesis of (−) sense RNA, an intermediary for the synthesis of new (+) strands that either serve as mRNA, replication template or genomic RNA destined for encapsidation into progeny virions.

The capsid-coding region of poliovirus type 1 (Mahoney; “PV(M)”) is re-engineered to increase toxic hexamer content. Synonymous encodings are synthesized with varying amounts of increased rare FD content, rare FI content, and combinations of rare FD and rare FI content, and are inserted into the PV(M) cDNA clone pT7PVM. Upon incubation with T7 RNA polymerase, the full length linear genomes produced above with all needed upstream and downstream regulatory elements yields active viral RNA, which produces viral particles upon incubation in HeLa S10 cell extract or upon transfection into HeLa cells. Alternatively, it is possible to transfect the DNA constructs directly into HeLa cells expressing the T7 RNA polymerase in the cytoplasm.

A modified influenza virus is engineered with a modified PB2 gene that has increased rare FD content while maintaining codon bias and without decreasing CPB (SEQ ID NO: 419). This sequence contains 0 of the lowest scoring H. sapiens CPS hexamers, and 7 of the lowest scoring H. sapiens FD hexamers. The FD bias of this sequence is -0.123, while the CPB of this sequence is 0.067.

Characterization of Modified Viruses

The functionality of each modified virus is then assayed using a variety of relatively high-throughput assays, including visual inspection of the cells to assess virus-induced CPE in 96-well format; estimation of virus production using an ELISA; quantitative measurement of growth kinetics of equal amounts of viral particles inoculated into cells in a series of 96-well plates; and measurement of specific infectivity (infectious units/particle [IU/P] ratio).

The functionality of each modified virus can then be assayed. Numerous relatively high-throughput assays are available. A first assay may be to visually inspect the cells using a microscope to look for virus-induced CPE (cell death) in 96-well format. This can also be run an automated 96-well assay using a vital dye, but visual inspection of a 96-well plate for CPE requires less than an hour of hands-on time, which is fast enough for most purposes.

Second, 3 to 4 days after transfection, virus production may be assayed using ELISA. The particle titer is determined using sandwich ELISA with capsid-specific antibodies. These assays allow the identification of non-viable constructs (no viral particles), poorly replicating constructs (few particles), and efficiently replicating constructs (many particles), and quantification of these effects.

Third, for a more quantitative determination, equal amounts of viral particles as determined above are inoculated into a series of fresh 96-well plates for measuring growth kinetics. At various times (0, 2, 4, 6, 8, 12, 24, 48, 72 h after infection), one 96-well plate is removed and subjected to cycles of freeze-thawing to liberate cell-associated virus. The number of viral particles produced from each construct at each time is determined by ELISA as above.

Fourth, the IU/P ratio can be measured.

To test the modified viruses as a vaccine, three sub-lethal dose of the virus are administered in 100 μl of PBS to 8, 6-8 week old CD155tg mice via intraperitoneal injection once a week for three weeks. A set of control mice receive three mock vaccinations with 100 μl PBS. Approximately one week after the final vaccination, 30 ul of blood is extracted from the tail vein. This blood was subjected to low speed centrifugation and serum harvested. Serum conversion against PV(M)-wt is analyzed via micro-neutralization assay with 100 plaque forming units (PFU) of challenge virus, performed according to the recommendations of WHO (Toyoda et al., 2007; Wahby, A. F., 2000). Two weeks after the final vaccination the vaccinated and control mice ware challenged with a lethal dose of PV(M)-wt by intramuscular injection with a 10⁶ PFU in 100 ul of PBS (Toyoda et al., 2007).

### Methods

Gene constructs were designed as described (Coleman et al., 2008) and were synthesized by Genscript. Sequences of all synthetic constructs are shown in Table S4, “Gene Sequences”. All HIS3 constructs were transformed into the native HIS3 locus of GZ238 (Zhao et al., 2016). Transformants were screened on SC-leu media, selecting for cotransformants containing a CRISPR/Cas9 LEU2 plasmid (Zhao et al., in preparation). All LYS2 strains were transformed into the BY4741 background with G418 selection, using fusion PCR cassettes containing the LYS2 gene and a KanMX6 or KanMX6-3HA marker (Longtine et al., 1998). All integrants were screened by PCR, and confirmed by Sanger sequencing. Deletion cassettes for UPF1, RQC1, DOM34, and SKIT were amplified from strains in the Yeast Knockout Collection (Winzeler et al., 1999) and transformants were screened for G418 resistance. Serial dilution experiments are 3-fold dilutions beginning with ˜16,500 cells per spot.

RNA was prepared using a RiboPure Yeast Total RNA Purification Kit. Northern Blotting was performed essentially as described in “RNA: A Laboratory Manual”, 2011 CSHL, with minor alterations. RNA Northern Blot probes were generated using T7 RNA Polymerase(NEB), with probe sequence directed against nucleotide regions common to all compared HIS3 or LYS2 gene alleles. Western Blotting was performed using standard methods using purified mouse primary antibody anti-HA 12CA5 from a hybridoma cell line. Chemiluminescence detection of Lys2 was achieved through ThermoFisher Goat 2° anti Mouse IgG₂b HRP conjugate. All quantifications of Northern and Western Blot signals used ImageJ. Ribosome profiling data were generated using the ArtSeq Yeast Ribosome Profiling kit with minor modifications described in (Gardin et al., 2014). Data is deposited at NCBI GEO with accession SRP044053.

Polysome fractionation was preformed on a diploid GZ238/GZ239 strain (Zhao et al., 2016) containing one copy of HIS3-FDF1-5 and one copy of wild-type HIS3, each at the native HIS3 locus. Cells were grown in 2 L SC-his liquid to a density of 2×10̂7 cells/ml. Cells were separated from media using a Whatman Filtering apparatus and 0.45μm cellulose filter papers, and immediately flash frozen in a 50 ml conical tube containing liquid nitrogen. 2 mml Polysome Lysis Buffer (20 mM HEPES pH 7, 100 mM KCl, 5 mM MgCl₂, 0.5% NP-40, 1 mM DTT, 100 μM cycloheximide, SUPERNAse-In 1 U/ml) was freshly prepared and added in small drops to the frozen cells in the 50 ml conical tube. Cells were disrupted using a TissueLyser II and stainless steel grinding jars for six 3 minute cycles at 15hz, recooling the grinding jars using liquid nitrogen after each cycle. 11.2 ml 15-55% sucrose gradients were prepared using a Hoefer SG15 gradient maker. Lysates were thawed in a 30° C. waterbath and clarified in a microfuge at max speed for 10 minutes. 400 μl supernatant was added to the gradient, and the gradient was spun at 35,000 rpm in a prechilled SW-41 rotor for 3 hours at 4° C. Gradients were fractioned using a peristaltic pump, injection needle, and UV absorbance monitor into a 96 well plate. 20 ng pTRI-B-Actin mRNA (AM7423) was added to each well as a spike-in control. RNA was purified using AmpPure XP Beads (2.1X v/v) and a 96 well magnetic bead separator with NEB ssRNA ladder (N0362S) added to monitor loss of small RNA molecules. Reverse transcription was performed with random hexamers and SuperScript III. qPCR was performed with LightCycler® 480 SYBR Green I Master mix in triplicate wells.

### REFERENCES

Brandman, O. et al., A ribosome-bound quality control complex triggers degradation of nascent peptides and signals translation stress. Cell 151, 1042-1054 (2012).
Cohen, B., and S. Skiena. 2003. Natural selection and algorithmic design of mRNA. J. Comput Biol. 10:419-432.
Coleman, J. R., et al., Virus attenuation by genome-scale changes in codon pair bias. Science 320, 1784-1787 (2008).
Coligan, J., A. Kruisbeek, D. Margulies, E. Shevach, and W. Strober, eds. (1994) Current Protocols in Immunology, Wiley & Sons, Inc., New York.
Gardin, J. et al., Measurement of average decoding rates of the 61 sense codons in vivo. Elife 3, (2014).
Gutman, G.A., and Hatfield, G.W., Nonrandom utilization of codon pairs in Escherichia coli. Proc Natl Acad Sci USA 86, 3699-3703 (1989).
Jayaraj, S., R. Reid, and D. V. Santi. 2005. GeMS: an advanced software package for designing synthetic genes. Nucl. Acids Res. 33:3011-3016.
Longtine, M. S., et al., Additional modules for versatile and economical PCR-based gene deletion and modification in Saccharomyces cerevisiae. Yeast 14, 953-961 (1998).
Mueller, S., et al., Live attenuated influenza virus vaccines by computer-aided rational design. Nat Biotechnol 28, 723-726 (2010).
Presnyak, V. et al., Codon optimality is a major determinant of mRNA stability. Cell 160, 1111-1124 (2015).
Quax, T.E., Claassens, N.J., Soll, D., van der Oost, J., Codon Bias as a Means to Fine-Tune Gene Expression. Mol Cell 59, 149-161 (2015).
Richardson, S. M., S. J. Wheelan, R. M. Yarrington, and J. D. Boeke. 2006. GeneDesign: rapid, automated design of multikilobase synthetic genes. Genome Res. 16:550-556.
Sambrook, J., E. F. Fritsch, and T. Maniatis. (1989) Molecular Cloning: A Laboratory Manual, 2^{nd} ed., Cold Spring Harbor Laboratory Press, Cold Spring Harbor, N.Y.
Shen, S. H., et al., Large-scale recoding of an arbovirus genome to rebalance its insect versus mammalian preference. Proc Natl Acad Sci USA 112, 4749-4754 (2015).
Skiena, S. S. 2001. Designing better phages Bioinformatics. 17 Suppl 1:5253-61.
Tian, J., H. Gong, N. Shang, X. Zhou, E. Gulari, X. Gao, and G. Church. 2004. Accurate multiplex gene synthesis from programmable DNA microchips. Nature. 432:1050-1054.
Wang, B., D. Papamichail, S. Mueller, and S. Skiena. 2006. Two Proteins for the Price of One: The Design of Maximally Compressed Coding Sequences Natural Computing. Eleventh International Meeting on DNA Based Computers (DNA11), 2005. Lecture Notes in Computer Science (LNCS), 3892:387-398.
Winzeler, E. A., et al., Functional characterization of the S. cerevisiae genome by gene deletion and parallel analysis. Science 285, 901-906 (1999).
Yang C., et al., Deliberate reduction of hemagglutinin and neuraminidase expression of influenza virus leads to an ultraprotective live vaccine in mice. Proc Natl Acad Sci USA 110, 9481-9486 (2013).
Zhao, G., Y. Chen, L. Carey, B. Futcher, Cyclin-Dependent Kinase Co-Ordinates Carbohydrate Metabolism and Cell Cycle in S. cerevisiae. Mol Cell 62, 546-557 (2016).

1. A modified protein encoding sequence comprising a polynucleotide sequence derived from a target protein encoding sequence, wherein the modified protein encoding sequence encodes a polypeptide having substantially the same amino acid sequence as the polypeptide encoded by the target protein encoding sequence and comprises a plurality of additional hexamers selected from one or more of the group consisting of SEQ ID NO:19 to SEQ ID NO:418 compared to the target protein encoding sequence.

2. The modified protein encoding sequence of claim 1, wherein the plurality of hexamers comprises frame independent hexamers.

3. The modified protein encoding sequence of claim 1, wherein the plurality of hexamers comprises frame dependent hexamers.

4. The modified protein encoding sequence of claim 1, wherein the modified protein encoding sequence comprises more than about 50 additional hexamers selected from one or more of the group consisting of SEQ ID NO:19 to SEQ ID NO:418 compared to the target protein encoding sequence.

5-9. (canceled)

10. The modified protein encoding sequence of claim 1, wherein the modified protein encoding sequence has reduced expression in mammalian cells compared to the unmodified protein encoding sequence.

11-12. (canceled)

13. The modified protein encoding sequence of claim 1, wherein the hexamers are introduced by rearranging synonymous codons of the target protein encoding sequence.

14. The modified protein encoding sequence of claim 1, wherein the hexamers are introduced by substituting synonymous codons of the target protein encoding sequence.

15. The modified protein encoding sequence of claim 1, wherein the target protein encoding sequence encodes a viral protein.

16. A modified virus comprising the modified protein encoding sequence of claim 15.

17. A method for reducing the expression of a target protein comprising introducing into the target protein encoding sequence a plurality of hexamers selected from one or more of the group consisting of SEQ ID NO: 19 to SEQ ID NO: 418 without altering the polypeptide sequence encoded by the target protein encoding sequence.

18. The method of claim 17, wherein the plurality of hexamers comprises frame dependent hexamers.

19. The method of claim 17, wherein the plurality of hexamers comprises frame independent hexamers.

20. The method of claim 17, wherein greater than about 50 hexamers are introduced into the target protein encoding sequence.

21. (canceled)

22. The method of claim 17, wherein hexamers are introduced by rearranging synonymous codons.

23. The method of claim 17, wherein hexamers are introduced by substituting synonymous codons.

24. The method of claim 17, wherein the target protein encoding sequence is a viral gene.

25. A modified protein encoding sequence comprising a polynucleotide sequence derived from a target protein encoding sequence, wherein the modified protein encoding sequence encodes a polypeptide having substantially the same amino acid sequence as the polypeptide encoded by the target protein encoding sequence and comprises at least one of: a plurality of frame dependent hexamers each having a frame dependent score less than about −0.51 and a plurality of frame independent hexamers each having a frame independent score less than about −0.33.

26-28. (canceled)

29. The modified protein encoding sequence of claim 25, wherein the modified protein encoding sequence comprises more than about 50 additional hexamers selected from one or more of the group consisting SEQ ID NO:19 to SEQ ID NO:418 compared to the target protein encoding sequence.

30-35. (canceled)

36. The modified protein encoding sequence of claim 25, wherein the modified protein encoding sequence has reduced expression in mammalian cells compared to the unmodified protein encoding sequence. 37-38. (cancelled).

39. The modified protein encoding sequence of claim 25, wherein the hexamers are introduced by rearranging synonymous codons of the target protein encoding sequence.

40. The modified protein encoding sequence of claim 25, wherein the hexamers are introduced by substituting synonymous codons of the target protein encoding sequence.

41. The modified protein encoding sequence of claim 25, wherein the target protein encoding sequence encodes a viral protein.


SEQUENCE LISTING (419 sequences)

SEQ ID NO: 1 - DNA, 666 bp, Saccharomyces cerevisiae
  misc_feature: WT His3
atgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtga

SEQ ID NO: 2 - DNA, 666 bp, Artificial Sequence
  HIS3scrambleallele1
atgacagagcagaaagccctagtaaagcgtattacaaatgaaaccaagattcagattgcgatttcgctgaaaggagggccgctggccatcgagcattccatatttcccgagaaggaagctgaggccgttgctgagcaagctacccaaagccaggtcatcaatgttcataccggaattggtttcctcgatcacatgattcacgcgctagcgaaacacagtggttggtctttgattgtagagtgcataggagatcttcatattgatgatcaccatactaccgaggattgtggtatcgcccttggccaggcgttcaaggaagcacttggtgctgtaagaggtgtcaagagatttggtagtgggtttgccccccttgatgaagcgctatctcgtgccgttgttgacctcagcaatcgaccctacgccgtggtggagctcgggctacagcgtgaaaaggttggtgacctttcctgcgaaatgattccacactttttagagtcgtttgctgaagcatcgaggataacacttcatgtagactgcttaaggggtaaaaacgaccaccataggtcggaatccgcatttaaagcgctggcagtagcaattcgtgaggcaacatcaccgaacggaacaaatgacgtaccttcgacaaagggcgtgctaatgtagtga

SEQ ID NO: 3 - DNA, 666 bp, Artificial Sequence
  dHis3allele1
atgacagagcagaaagccctagtaaagcgtattacaaatgaaaccaagattcagattgcgattagccttaaggggggtcccctggctatagaacactcgatttttccagaaaaagaagccgaggccgtagccgaacaagcgacccagagtcaggtaattaatgtgcataccgggataggttttctagatcatatgatccacgcactggctaaacactcgggttggagccttatagttgagtgtataggagacttacatattgacgatcaccatacaaccgaggattgcggaatcgcactaggtcaggcttttaaggaagccttaggcgcggttaggggggttaaacgttttggttcgggtttcgcccctctagacgaggctctgtctagagcggttgtagacttgtctaatagaccctacgcagtagttgagttgggactccaacgtgagaaggtaggcgatcttagttgcgagatgatcccccactttcttgagtccttcgccgaggcctcacgtattaccctccacgtggattgccttaggggtaaaaacgatcatcatagatccgaatcggcttttaaggcactcgcagtcgcgattagggaagcgacctccccgaatggtacaaacgacgtcccgtcgactaaaggagtgcttatgtagtga

SEQ ID NO: 4 - DNA, 666 bp, Artificial Sequence
  dHis3allele2
atgacagagcagaaagccctagtaaagcgtattacaaatgaaaccaagattcagattgcgattagccttaaggggggtccactcgcgatcgaacactcgatttttcccgagaaagaagccgaggccgtagccgagcaagcaacccagtcccaagtaattaatgtgcataccggaatcggttttctagatcatatgattcacgcgttagctaaacactcgggttggtctcttatagttgagtgtataggtgatctacatatagacgatcaccatacaaccgaagactgcggaatcgctctgggtcaggcttttaaggaggctctaggcgcggttaggggggttaaacgtttcgggtcgggttttgcacccctagacgaagcacttagccgtgcagttgtagaccttagtaaccgaccgtacgcagttgtagaactcggcttgcagagagagaaggtgggtgatctgtcttgcgagatgattccccactttctcgaatcgttcgcggaagcgagtagaattacattgcatgtcgattgccttaggggtaagaatgatcaccatagatccgagtcagcctttaaagccttagccgtagctatacgtgaggcgacctcccctaacggaaccaatgacgtcccgtcgacaaaaggagtgcttatgtagtga

SEQ ID NO: 5 - DNA, 666 bp, Artificial Sequence
  His3FDF1allele1
atgacagagcagaaagccctagtaaagcgtattacaaatgaaaccaagattcagattgcgatctctttaaaaggcggtccactcgcaatcgaacactcaatctttccggaaaaggaagcagaagcagtcgccgaacaagcgacccaaagtcaggtaattaacgtgcataccgggattggttttctggatcatatgatacacgcactcgcaaaacatagtggctggtcccttatcgtggagtgcattggagaccttcacattgatgatcatcataccaccgaagattgcggaatcgccttgggtcaggcctttaaggaagcactcggagcggttaggggcgtaaagaggttcggatcgggtttcgccccgcttgatgaggccttgtcgagagccgtagttgatcttagcaatcgaccctacgcagttgtagaacttggtctccagagagagaaggtaggggacttgtcttgcgaaatgataccacactttcttgagtcgttcgctgaagcgagtagaataacacttcacgttgactgcctgaggggcaaaaatgaccaccacagaagtgagtccgcttttaaagcactcgccgtagctataagagaagcaacctccccgaacggtaccaacgacgtcccgtccaccaaaggtgttcttatgtagtga

SEQ ID NO: 6 - DNA, 666 bp, Artificial Sequence
  FDF1His3allele5
atgacagagcagaaagccctagtaaagcgtattacaaatgaaaccaagattcagattgcgatctctcttaaaggcggtccactggctatagagcattcaatctttccggaaaaggaggccgaagctgtcgctgaacaagctacccaaagccaagttataaacgtgcataccgggattggttttctggatcatatgatccacgcccttgctaaacactcgggatggtccctcatcgtcgaatgtatcggggatcttcatattgatgaccaccataccaccgaagattgcggaatcgccttgggtcaggcgttcaaggaagcactcggagcggttagaggcgtaaaaagattcggatcgggtttcgccccgcttgatgaagcgttgtcgagggcagttgtagatttatctaataggccttacgccgtagttgagttgggtcttcagagagagaaagtaggagacctctcttgcgaaatgatacctcactttttagagagtttcgccgaagcatcgaggataactctccacgttgactgccttaggggtaaaaacgatcaccacagaagcgagagcgcatttaaagccctggccgtcgctataagagaggctacctccccgaacggcacaaatgacgttccatccaccaaaggtgttcttatgtagtga

SEQ ID NO: 7 - DNA, 666 bp, Artificial Sequence
  His3FDF23allele
atgacagagcagaaagccctagtaaagcgtattacaaatgaaaccaagattcagattgcgatcagcctcaagggtgggccgttggcaattgaacacagcatattccccgagaaggaggctgaagccgtggcggagcaagccacacaatcccaggttatcaatgttcacaccggaattggatttttggaccacatgattcatgcacttgccaaacacagtgggtggagccttattgtggagtgtataggggatcttcacatcgacgaccatcacaccaccgaggattgtggtatagcgttaggacaagcctttaaagaagcgctgggtgctgtacgaggtgtgaaacgatttggcagtgggtttgctccgttggatgaagcgctgtcgcgtgcggtcgtagatctttcgaataggccgtacgcggtggtggagctgggattgcaaagggaaaaagttggcgacctgtcgtgtgagatgattccccattttcttgaatcgtttgcggaggcttcgcgtataacccttcatgttgattgccttcgtggaaaaaatgaccatcacagaagtgagagtgcgttcaaggcgcttgcggtggccattcgtgaggcaacatctcccaacgggactaatgatgtcccctccaccaaaggtgttcttatgtagtga

SEQ ID NO: 8 - DNA, 666 bp, Artificial Sequence
  His3FIF1allele1
atgacagagcagaaagccctagtaaagcgtattacaaatgaaaccaagattcagattgcgatcagcttaaaaggtggtcccctggcgatcgagcactctatatttccggaaaaagaggccgaggctgttgccgagcaagcaacccaaagtcaggtaatcaatgttcataccggtattggatttttggaccatatgatccatgcgcttgcgaagcactcgggttggagcctcattgtcgagtgtattggtgatctacacattgacgaccaccacaccaccgaggattgtggtatcgcgcttggtcaggcctttaaagaagcacttggcgctgtccgtggggtcaaaaggtttgggagtgggttcgctcccctggatgaggccttaagtcgtgcggttgtagacctaagcaataggccctacgccgttgtagaacttggcctccaaagggaaaaggttggtgatctaagctgtgagatgataccacatttccttgagtcttttgccgaggcgtcgcgtataacattgcacgtggactgcctcagaggtaagaacgaccatcacagatccgaatcggcttttaaggctctagctgtagctattcgagaagcaactagccccaacggcactaatgatgtcccctccaccaaaggtgttcttatgtagtga

SEQ ID NO: 9 - DNA, 666 bp, Artificial Sequence
  His3FIF23allele1
atgacagagcagaaagccctagtaaagcgtattacaaatgaaaccaagattcagattgcgatctctttaaaggggggcccgcttgccattgagcacagcatatttcctgaaaaagaggcggaagccgtagcggaacaagcaactcagagtcaagttattaacgttcataccggaatcggttttttggaccatatgattcacgctctagcgaaacacagtggatggtcacttattgttgaatgcataggggacttgcatatcgatgatcaccataccaccgaagattgcggcattgcccttggacaagcctttaaggaagcactgggagctgtaagaggtgttaagagattcggtagcggattcgccccactggatgaagctctttcgagggccgtcgtagatctttctaataggccgtacgccgtcgttgagcttggtctacaaagggagaaagtaggcgatctctcgtgtgaaatgataccccactttcttgagtcgtttgcggaagccagtagaattaccctgcacgttgactgcctaagagggaaaaacgatcatcacaggagcgagtcggcatttaaagcactcgcagtcgcgattagagaagctacctctcccaacgggaccaatgatgtaccgtccaccaaaggtgttcttatgtagtga

SEQ ID NO: 10 - DNA, 666 bp, Artificial Sequence
  His3CodonBiasDeoptimizedallele1
atgacagagcagaaagccctagtaaagcgtattacaaatgaaaccaagattcagattgcgatctcgctcaagggggggccgctcgcgatcgagcactcgatcttcccggagaaggaggcggaggcggtggcggagcaggcgacgcagtcgcaggtgatcaacgtgcacacggggatcgggttcctcgaccacatgatccacgcgctcgcgaagcactcggggtggtcgctcatcgtggagtgcatcggggacctccacatcgacgaccaccacacgacggaggactgcgggatcgcgctcgggcaggcgttcaaggaggcgctcggggcggtgcggggggtgaagcggttcgggtcggggttcgcgccgctcgacgaggcgctctcgcgggcggtggtggacctctcgaaccggccgtacgcggtggtggagctcgggctccagcgggagaaggtgggggacctctcgtgcgagatgatcccgcacttcctcgagtcgttcgcggaggcgtcgcggatcacgctccacgtggactgcctccgggggaagaacgaccaccaccggtcggagtcggcgttcaaggcgctcgcggtggcgatccgggaggcgacgtcgccgaacgggacgaacgacgtgccgtccaccaaaggtgttcttatgtagtga

SEQ ID NO: 11 - DNA, 4179 bp, Saccharomyces cerevisiae
  misc_feature: WT Lys2
atgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagttcgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaa

SEQ ID NO: 12 - DNA, 4179 bp, Artificial Sequence
  LYS2scramble
atgactaacgaaaaggtctggatagagaagttggataatccaactctttcagtgttaccacatgactttttacgcccacaacaagaaccttatacgaaacaagctacatattcgttacagttgccacaattagacgttccacacgattcattcagcaataaatatgccgttgccctttccgtttgggccgctttaatttaccgtgttactggtgatgatgatatcgtcttatacattgccaacaataaaattttgaggtttaacatccaacctacctggtctttcaatgaattatattccaccatcaataatgaactaaataaattgaacagcatagaagcaaacttcagtttcgatgaattggcagaaaagatccaatcatgtcaagatttagaaagaacaccacaattatttagattagcgttcctagaaaatcaagattttaaactggatgaatttaaacatcatttggttgattttgcgctaaatttagacacttcaaacaatgctcacgtactaaatttgatatacaattctcttttatactccaatgaacgtgttactattgtggcagatcaattcacccaatatttaacggcggctttgtctgacccatctaactgtattaccaagatttcattaattactgcctcttctaaagattcgctgccagatcctaccaaaaatttaggttggtgtgattttgttggttgcatacatgatatatttcaagataatgcagaggcatttcctgaaaggacatgcgttgttgaaacaccaactttgaacagtgataaatcaagatcgtttacctacagagatattaatagaacaagcaatattgtggcacattatttaatcaagaccggtattaagcgtggtgacgttgttatgatttattcatcaagaggtgttgatttaatggtttgtgttatgggtgttttaaaggctggtgccactttcagtgtcattgatccagcttacccacctgcaaggcaaacaatatatttgggtgttgccaagccaagaggtttaatcgtcatcagggccgccggtcaattagaccaattggttgaagactatattaatgatgagctagaaatagtatctcgtattaacagtattgccatccaagaaaatgggacaatagaaggtggtaagctagataatggtgaagatgttttagcaccttatgaccattataaggatacacgcaccggtgttgttgttggtcctgacagtaatccaactttgagctttacttcaggaagtgaaggaataccaaaaggtgtcttgggccgccatttcagtttagcgtactattttaactggatgtcgaagagatttaatttgactgaaaatgacaagtttaccatgttatctggtattgctcacgaccctatccagcgtgacatgttcactccacttttcttgggtgctcaactatacgttccaacccaagatgacatcggcactccaggtagattggcagaatggatgtccaaatatggctgtacggtaacgcatttgactccagcaatgggccaactgttaacagcgcaggccaccactccatttccaaaactgcaccatgcatttttcgttggtgatatcctcaccaaaagggactgcttaagattgcaaactttagctgaaaactgtagaattgtcaacatgtatggaacaacagaaactcaaagagctgtttcatattttgaagtcaagtccaagaacgatgacccaaatttcttaaagaagctaaaggatgttatgccagcaggaaagggtatgctaaatgttcaattgttggtggtaaatagaaatgacagaacgcaaatttgcggaatcggtgaaattggggaaatatatgtcagagctggtggtttagcggagggctaccgtggtttgccagagctaaacaaggagaaatttgttaataattggttcgtggagaaggaccattggaactatttagacaaagataatggagagccatggaggcaattttggttggggcctcgtgaccgtctttatcgtactggtgacctgggaagatatttgccaaatggcgattgtgagtgctgtgggcgagcagatgaccaagtcaagatcagaggttttcgcattgagttgggtgaaattgacactcatatatcacagcatcctttagttcgtgaaaatataacgctggtaagaaagaatgcggataatgaaccaactttaattactttcatggttccaaggtttgataaacctgatgatttatccaaatttcaatcagatgtgcccaaagaagtggaaacagatccaattgtcaaaggtttgattggatatcatttactttctaaagatattcgcacatttttgaaaaagaggctagcctcttatgccatgccatctttaattgttgtcatggacaagttgccattaaacccaaatgggaaagtggacaaaccaaagttacagtttccaacaccaaaacaattgaacttagttgcagagaacaccgtcagcgagacagatgattcgcaattcacaaatgtagaaagagaagtacgtgatctttggctttccattcttccaacaaaacctgcttccgtttctcctgatgacagtttctttgatttgggtggccactccattttagccacaaagatgatattcactttgaaaaagaaattacaagtggacttgcccttaggtaccatctttaaatatccaaccattaaagcatttgctgctgaaatagatcgtatcaaatcttctggtggttcttcacaaggtgaagttgttgagaacgttaccgccaactatgctgaagatgctaaaaagctagtggaaactttgccttcatcatatccatcaagggagtacttcgtggagccaaactccgctgaaggtaagaccaccatcaacgttttcgtcactggtgttactggtttcttaggttcatatattttagcggaccttttgggtagatcgcccaaaaattattcctttaaagtttttgcgcatgttcgtgcaaaagatgaggaggcagcttttgcccgtctgcagaaagctggcattacatatggaacgtggaatgaaaagtttgcttctaacatcaaggttgttttgggtgacctttccaaatcacagtttgggttaagtgatgaaaaatggatggatttagccaatactgtagatattatcattcacaacggtgctttggtgcattgggtatatccttatgctaaactaagagacccaaacgtcatcagtaccattaacgtcatgtctttggctgctgttggtaaacccaaatttttcgattttgtttcttctaccagcacgttagatacagaatattatttcaacctcagtgacaagttggtatctgaaggtaaaccaggtatcttggaaagtgatgatttgatgaatagcgcctctggtttaacaggcggttacggccaaagtaaatgggcggcagaatatattatcaggagagctggtgaaagaggtttgagaggttgcatagttcgtcctggctatgttactggtgcttccgcaaatggaagcagcaatactgatgattttttgttacgcttcctcaagggctctgttcaactgggaaaaataccggacattgaaaactctgttaatatggtacctgttgaccacgttgccagggtggtggtggccacctcattaaacccaccaaaggagaatgagctagctgtagcgcaagttactggccatcctcgtattctttttaaggattatttatacacgctgcatgactatggatatgatgttgaaattgaatcttattccaaatggaaaaaaagtttagaagcttccgtaatagatagaaatgaggaaaatgcactatacccattattgcatatggttttggataatttgccagaatccacaaaggcgccagaattggatgacagaaatgctgttgccagcttgaaaaaggatactgcttggactggtgtcgactggtccaatggtattggtgtcacacctgaagaagttggtatttacatcgccttcttgaataaagtaggtttcttgccaccaccaacacacaatgataaattaccattaccttccattgaattgacccaagcacaaattagcttagtcgcgtccggagccggtgctcgtggttcctcagccgcggcttaa

SEQ ID NO: 13 - DNA, 4179 bp, Artificial Sequence
  dLYS2-2
atgactaacgaaaaggtctggatagagaagttggataatccaactctttcagtgttaccacatgactttttacgcccacaacaagaaccttatacgaaacaagctacatattcgttacagttgccacaattggatgttccacacgatagcttctctaataagtacgcagtcgcgttgtcagtctgggcagctctgatttatagagttacaggcgacgatgacatcgtgttatatatagctaataataagattttgcgttttaatattcaacctacttggtcttttaacgaattatactcgactattaataacgaacttaataagttaaattctatagaagctaatttctctttcgacgaattagccgagaagattcagtcttgtcaagacttagagagaaccccacaattgtttagattagctttcttagaaaatcaagactttaagttagacgaatttaaacatcacttagttgatttcgctttaaacttagatacatctaataacgctcatgtgttaaacttaatctataattccttgttgtatagtaacgaaagggttacaatcgtagccgatcaatttacacaatatctaaccgcagccttgtctgaccctagcaattgcattactaaaattagcttaattacagcctcatctaaagatagcttgccagaccctactaagaatctgggttggtgcgacttcgtaggttgtattcatgacatttttcaagataacgccgaagcttttccagagagaacttgtgtagtcgaaacccctacattgaattccgataagtcacgtagctttacttatagagacattaatagaacgtctaacatagttgcacactatctaattaagactggcattaagagaggggatgtagttatgatttactcgtctagaggggttgacttaatggtgtgtgttatgggagttctaaaagccggagctacattctcagttatagatccagcctaccccccagctagacaaacaatctatttgggagtcgctaaaccacgcggtttaatagttattcgcgccgcgggtcagttggatcaattagttgaagactatattaatgatgaattagaaatcgtgtcacgtattaactcaatcgctatacaagaaaatggtacgattgaggggggtaagttagataacggcgaagatgttctagccccttacgatcattataaggatactagaaccggagtcgttgtaggtccagactcaaatccaaccttgtcttttactagcggttccgaaggaatccctaagggagttctaggtaggcatttctctctggcttattactttaattggatgtctaaacgttttaacttaactgagaatgataagtttactatgttgtccggcatcgcacacgatccaatccaacgtgatatgtttactcctttgtttctaggtgcacaattgtatgtcccgacccaagacgatataggtacaccaggtaggttagccgagtggatgtctaagtatggttgtacagttacacacttaaccccagctatgggtcagttgttaaccgcacaagcaacgactccttttccaaaattgcatcacgcattcttcgtaggtgatatcttaactaaaagagattgcttgcgtttgcagaccttagccgaaaattgtagaatcgttaatatgtatggtacaaccgaaacacaacgcgcggtctcatacttcgaggttaagtctaagaatgatgatccaaatttcttaaaaaaacttaaggatgttatgccagccggcaaaggcatgttaaacgtgcaattgttagtagttaatcgtaacgatcgtacacaaatttgcggaatcggtgagataggtgagatttatgttagagccgggggtttagccgagggttatagaggcttgccagaacttaataaagaaaaattcgttaataattggttcgttgaaaaagatcattggaattatctagataaagataacggtgaaccttggcgtcaattttggttgggtcctagagatagattgtatagaaccggagacttaggtaggtacttaccaaatggtgattgcgaatgttgcggtcgcgcggatgatcaagttaagattaggggttttcgtatagaattaggcgaaatcgatactcatattagtcaacaccctttagttagagaaaacattacattggtgcgtaaaaacgccgataacgaaccaacattgattacctttatggttccacgttttgataaaccagacgatctgtctaagtttcaatccgacgtccctaaagaagttgagactgacccaatcgttaaaggcttgataggttatcacctattgtctaaagatattcgtacattcttaaaaaaaagactcgcatcctatgctatgccatccttaatcgtagttatggataagttgccacttaatccaaatggtaaggttgataaaccaaaattgcaatttccaacccctaaacaacttaatctagttgccgaaaatactgtgtctgagactgacgactcacaatttactaatgtcgagagagaggttagagacttgtggttgtcaatcttgcctactaaaccagctagcgttagtccagacgattctttttttgacttagggggtcattcaattttggcaactaagatgatttttacattaaaaaaaaaattgcaagttgacttaccattaggtacaatctttaagtatccaacgattaaagctttcgcagccgagattgataggattaagtcctccgggggtagttcccagggtgaggttgtagaaaacgttacagctaattatgccgaagacgctaaaaagttagttgaaacattgcctagctcctatccatcacgtgaatatttcgttgaacctaactcagccgagggtaaaacgactattaatgtgttcgtaaccggagttacgggttttctaggtagttacattctagccgatctgttaggtaggagtccaaaaaattactcttttaaggtgttcgctcatgttagagctaaagacgaagaagcagctttcgctaggttgcaaaaagccggaattacttatggtacatggaacgaaaaattcgctagtaatattaaggtggttctaggtgacttgtctaagagtcaattcggtctgtccgacgaaaagtggatggatctggctaatacagttgacattattatacataatggtgcattagttcattgggtgtacccttacgctaagcttagagatccaaacgtgattagtactattaacgttatgtcactcgcagccgtaggtaaaccaaaattttttgatttcgtgtctagtacgtctacattggataccgaatattactttaatttgtccgataagttagttagcgaaggtaaaccaggcattttggagtccgacgatcttatgaattcggcttcgggtctgactggaggttatggtcagtctaagtgggcagccgaatatattattagacgcgcgggtgagaggggtcttaggggttgtatagttagaccaggttatgtaaccggcgcgtccgctaatggtagttctaataccgacgactttctacttagattcttaaaaggttcagtccaattaggtaaaatcccagatatagaaaactcagttaatatggttccagtcgatcatgtcgcgagagttgtagtcgcgacctcccttaatccacctaaagaaaacgaattagctgtagctcaggttacgggtcaccctaggatcttgtttaaagattacttatataccctacatgattacggttatgacgtcgaaatcgaatcttactctaagtggaaaaagtccttggaagcatcagttatagataggaatgaagaaaacgcattgtatccattgttacatatggttctagataacttacccgaatcgactaaagcccctgaattagacgatcgtaacgcagtcgcgtctcttaaaaaagatacagcttggactggagtcgattggtctaatggtataggtgtgactcccgaagaggttggcatctatatagctttcttaaataaggtgggttttttgccacccccgacccataatgataagttgccattgcctagcattgagttgacacaagcacaaattagcttagtcgcgtccggagccggtgctcgtggttcctcagccgcggcttaa

SEQ ID NO: 14 - DNA, 4384 bp, Artificial Sequence
  dLys2-4HA
atgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgaattagccgaaaaaatccaatcctgtcaagacttagaacgtacaccacaattgtttcgtttagcttttctagaaaatcaagactttaagttagacgaatttaaacatcacctagttgacttcgcacttaatttggatacgtctaataacgcacacgtgttaaatctaatttataattccttgttgtattctaacgagagagttacaatcgtagccgatcaatttacacaatatttgacagctgcattgtccgatccatctaattgtattactaagattagtctaattacagctagttctaaagactcattgccagatccaactaaaaacttaggttggtgcgatttcgtaggttgtattcatgatatttttcaagataacgccgaggcttttccagaacgtacttgcgtcgttgagacccctactcttaattccgataagtctagatcttttacttatagagacattaatcgtacgtctaatatcgtagctcattacttaattaaaaccggcattaaacgcggtgatgtagttatgatctatagttctagaggagtcgatctaatggtttgcgttatgggagtcttaaaagccggcgcgaccttctcagttatagatccagcttatccacccgcgagacagactatttacttaggcgtagctaaacctaggggtctgatcgtaattagagccgcgggtcagttagatcaattagttgaagattacattaacgacgaattagaaatagttagtagaattaattctatagctatacaagaaaacggtacaatcgaagggggtaaattggataacggtgaagacgtgttagcaccatatgatcattataaagatactcgtacaggtgtagttgtaggtccagactctaatccaacattgtcttttacatcgggttccgaaggcatccctaaaggcgtgttaggtaggcactttagcttagcttattactttaattggatgtctaaacgttttaatctgaccgaaaacgataagtttactatgttgtcgggtatagctcatgatccaattcagagagatatgtttacaccattatttttaggtgcccaattgtatgttccaacacaagacgatataggtaccccgggtaggttagccgagtggatgtctaagtatggttgtacagttacacacctaactcccgcgatgggtcagttgttgacagctcaggctacgaccccttttcctaagttacatcacgcttttttcgtaggtgatattctgactaaacgtgattgtcttagactccaaaccctagccgagaattgtagaatcgttaatatgtatggtacgactgagactcagagagcagttagttatttcgaagttaagtctaaaaacgacgatccaaattttcttaaaaaattaaaagacgttatgccagcgggtaaaggcatgttaaatgttcaattgttagttgttaatcgtaacgatagaactcaaatttgcggtataggtgagataggtgagatctatgttagagccgggggtttagccgagggttataggggtctgccagaattaaataaagaaaaattcgttaataattggttcgttgaaaaagatcattggaattacttagataaggataacggcgaaccatggcgtcaattctggttaggtcctagagatagattgtatagaaccggcgatctaggtaggtatttgcctaacggagattgcgaatgttgcggtcgagccgacgatcaagttaagattaggggttttcgtatagaattaggtgagattgatactcatattagtcaacaccctctagttagggaaaatattaccctagttagaaaaaacgccgataacgaaccaaccctaattacctttatggtccctagattcgataagccagacgacttgtctaagtttcaatccgacgtccctaaagaagtcgagaccgatccaatcgttaaaggtttaataggttatcacttgttgtctaaagacattagaacctttctaaaaaaacgcttggctagttacgctatgcctagcttgatcgtagttatggataagttgccacttaatccaaacggtaaagtcgataagcctaagttgcaatttccaacccctaaacaacttaatttagtagccgagaatacagtttccgaaaccgacgatagtcaatttactaacgttgaacgcgaagttagggatttgtggttgtctattttgcctactaaaccagcctcagttagtccagacgatagcttcttcgatctagggggtcatagtatcttagcaactaagatgatctttactcttaaaaaaaaattgcaagttgatctgccactaggtacgatttttaagtaccctactattaaagctttcgcagccgagattgatagaattaagtcttccgggggttctagtcagggtgaggtagttgagaatgttacagctaattacgccgaagacgcaaaaaagttagttgagaccttgccatctagctaccctagtagagaatatttcgttgaaccaaattcagccgagggtaaaactacgattaacgtgttcgtgactggagttacaggttttctaggttcctatatcttagctgacttgttaggtaggtcccctaagaattatagctttaaggtgttcgctcatgttagagctaaagacgaagaagccgcattcgctagactccaaaaagccgggattacatacggcacttggaacgaaaaattcgcgtctaacattaaggttgtgttaggcgatctgtctaagtcccaattcggcttgtccgacgaaaagtggatggacttagctaatacagttgatattattattcataatggtgcgttagttcattgggtgtatccatacgcaaaattgcgtgatccaaatgtgatctctacgattaatgttatgtccttagccgcggttggcaaacctaagttcttcgacttcgttagctcaacctcaaccctagataccgaatattactttaatttgtccgataagttagtctcagagggtaaaccaggaatcttagaatccgacgacttaatgaattcagcctcgggtctgaccgggggttatggtcagtctaagtgggcagccgaatatataattagacgcgcgggtgaacgcggtcttaggggttgtatcgtgcgtccaggttatgttacaggtgcatcggctaatggttcgtctaatactgacgatttcttgttgcgttttcttaaaggttcagtccaattaggtaagattccagacattgagaattcagttaatatggtgccagtcgatcacgtagctagggttgtagtcgcgactagcttaaatcccccaaaagaaaacgaattagcagttgcacaagttacaggtcaccctagaattttgtttaaggattacttatatacattgcatgattacggttatgatgttgagattgagtcctatagtaagtggaaaaaatccctcgaagcctcagttatagatcgtaacgaagaaaacgcattgtaccctttgttacatatggtgttagataatttgccagaatcaactaaagcaccagaattagacgatcgtaacgcagtcgcgagcttaaaaaaagatacagcttggaccggagtcgattggtctaacggaatcggagttacaccagaagaagttggcatttatatagcttttcttaataaggtgggttttctgccacccccgacccataacgataagttgccattgccatcaatcgaattgacacaagcacaaattagcttagtcgcgtccggagccggtgctcgtggttcctcagccgcggcttaaggttgagcattacgtatgatatgtccatgtacaataattaaatatgaattaggagaaagacttagcttcttttcgggtgatgtcacttaaaaactccgagaataatatataataagagaataaaatattagttattgaataagaactgtaaatcagctggcgttagtctgctaatggcagatcgattgtcgactgcagcggccgc

SEQ ID NO: 15 - DNA, 4179 bp, Artificial Sequence
  Lys2FDF1
atgactaacgaaaaggtctggatagagaagttggataatccaactctttcagtgttaccacatgactttttacgcccacaacaagaaccttatacgaaacaagctacatattcgttacagctaccccaattggacgttccacatgactctttttctaataaatacgctgttgcactgtcagtttgggccgccttgatctatagggttacgggtgacgatgatattgtactttatatcgctaataacaaaatcttgcgattcaatattcaaccaacttggtcttttaacgagttgtattctaccattaataatgagttgaataaactaaatagtattgaagcaaacttctcttttgacgaattggctgagaaaattcaatcctgtcaagacttagaacgtactccacaattattcaggttagcttttttagagaatcaagactttaagttggacgaattcaagcaccacctagttgatttcgccttgaatttggatacttctaataacgcccacgtgttaaatttaatttataatagtttattatacagtaacgaaagagttacaatcgtagcggatcagtttacacaatatttaaccgcagcgctatccgatccatccaattgcattactaaaatcagtctaataactgcgagttccaaagatagcttgccagatcctaccaagaatttaggatggtgtgattttgtaggctgcatccacgatatctttcaagataatgctgaagcatttccagaacgtacctgcgtggttgagacaccaactctaaattctgataaatcccgtagctttacatatagagatattaaccgtacgtctaacatcgttgctcattatctgattaagacgggcattaaaaggggtgacgtggttatgatttattctagcagaggtgtagatttaatggtttgtgttatgggcgttctaaaagccggggccacattctctgttattgatccagcctacccgccagctaggcagactatatacttgggcgtcgctaagccaagaggcttgatagttattagagccgcgggtcaacttgaccaattagtcgaagactacataaatgatgagttggaaatagtttcccgtataaattcaatcgcgatacaagaaaacggtacaatcgaaggaggtaaattggataatggtgaagatgtattggccccatatgatcattataaggatactaggactggagttgtcgtgggtccagattctaaccctacattatcattcacctcgggttcggaaggtattcctaagggtgttttagggaggcactttagcttagcctattactttaactggatgagtaagaggttcaatttaaccgaaaatgataaatttactatgttgtcaggcatcgcccacgatcctatccaaagggacatgttcactccattgttcttaggggctcaactgtatgttccgacacaagacgatataggtacaccaggacgtcttgccgaatggatgtctaaatacggctgtacagttacacacctcacccctgctatgggtcaattgttgactgctcaagcaaccaccccttttccaaaactacaccacgcattcttcgttggtgatattttaacaaaaagagactgccttaggttgcagactttggccgaaaattgtagaatcgttaatatgtacggcacaactgaaacccagagagcagttagctactttgaggttaaatcaaagaatgacgatccaaattttttgaaaaagttgaaagatgttatgccagctggtaagggaatgttgaacgttcaattgttagtcgttaataggaatgacagaacccaaatttgcggtattggtgaaattggtgaaatctatgttagagcgggtggtttggccgaaggttaccgaggtttgccagaactaaataaagaaaagttcgttaataactggttcgtggagaaggatcactggaattacttagacaaggacaacggtgaaccatggcgtcagttttggttgggtcctagagatagactatacagaacaggtgacctggggcgttatttaccaaacggtgactgcgaatgctgcggtagggccgatgatcaagttaaaatcagaggttttagaattgaacttggagaaatcgatacccatattagtcagcatccattagtgagagaaaacatcaccttagtgagaaagaacgccgacaatgaaccaactcttatcacgtttatggttcctagattcgataagccagacgatctaagcaaatttcagtcagatgttcctaaggaagtcgaaaccgatccaatcgttaaaggtttaataggttatcacctattgtccaaggacattaggacatttttaaaaaagcgtttagcatcttacgctatgccatcacttatcgttgttatggataagttgccattgaatcctaacggtaaggtcgataagcctaagttacaatttccaacaccaaaacaattgaacctcgttgctgaaaacaccgttagtgaaactgatgattcacaatttactaacgttgaaagagaagttagagatttgtggctgtctattttgccaactaaaccagcttcagttagtccagatgattctttctttgatctaggtggtcattcaatcttagccactaagatgatctttacattaaaaaaaaagttgcaggttgatttaccattggggacgatttttaagtatccaactattaaagctttcgccgcggaaattgatagaattaaaagttccgggggttcaagtcagggcgaagtagttgaaaatgtcaccgccaactacgcagaggacgcaaaaaagttggtagaaacattaccatccagttatccatctagagaatacttcgttgaaccaaattcagccgaaggcaaaactactattaatgttttcgttacaggtgtgaccggttttttgggttcctatatattagccgacttattgggaaggtccccaaagaattactcttttaaagttttcgcgcatgttagagcaaaagacgaagaagcagctttcgctcgtcttcaaaaagcaggtatcacttacggtacgtggaatgaaaaatttgctagcaatataaaagtcgtgctaggtgatttatctaagtctcaattcggtttgtctgatgaaaagtggatggatttagctaacactgtagatatcattattcacaacggtgccttggttcactgggtgtacccctatgctaagttaagagacccaaacgtaatctctaccataaacgttatgtcattagcagcagttggaaaacctaaattttttgacttcgtgtctagcacttctaccttggatacggaatattattttaatctgtctgacaaattagtatctgaaggtaagccaggtattttagaatccgatgatttaatgaattcagcttccggcttgaccggcggctacggtcagagcaagtgggccgccgaatacattattcgtcgcgcgggtgagagaggtttgcgtggttgcatcgttagaccaggttatgttacaggtgcttccgctaacggttcatccaacacagatgacttcttattgcgttttttgaaaggtagcgtacaattgggaaaaattccggatattgaaaattccgttaatatggttccagttgatcatgtagccagagtagttgttgctacctctttaaacccaccaaaagaaaacgaactggccgtggcccaagttacaggtcatccaagaatcttatttaaggattacttatatacattacatgattacggttatgatgtcgaaattgaatcatactctaagtggaagaagtctttagaagcaagcgttattgaccgtaatgaagaaaatgctttgtatccacttctacatatggtcttagataatttacctgagagcacaaaagcacccgaactagatgataggaatgctgtcgcgtcccttaaaaaagacactgcttggaccggtgtcgattggtcaaacggtatcggcgttaccccagaagaagtcgggatctatatcgcttttttgaataaggttggatttttacctccaccaactcataatgacaaacttccactgccaagtatagaactaactcaagcgcaaataagtctagttgcttcaggtgctggtgctcgtggaagctccgcagcagcttaa

SEQ ID NO: 16 - DNA, 4179 bp, Artificial Sequence
  Lys2FDF23
atgactaacgaaaaggtctggatagagaagttggataatccaactctttcagtgttaccacatgactttttacgcccacaacaagaaccttatacgaaacaagctacatattcgttacagctacctcagttagatgttcctcatgactcattcagtaataagtatgccgttgcattgagtgtttgggctgcacttatttacagggtcactggtgacgacgatattgttttgtacattgcaaacaacaaaattctccgtttcaacattcagccaacttggtcgttcaatgaattatactccacaataaacaatgagttgaacaagttaaattccattgaagccaatttttcctttgacgaattggccgagaagattcaaagttgccaagatttggagagaaccccacagcttttccgcttggcctttttagaaaatcaagatttcaaattggacgaatttaagcatcatctagttgactttgccctgaacttggatacctccaacaatgctcacgttttaaatttgatatataactctttactttattctaatgaacgtgtcactattgttgcggaccagtttacgcaatatctaactgctgccttatcagatccatctaactgtattacaaaaatttcgttaattaccgcttcctccaaagattcacttccagacccaaccaaaaaccttgggtggtgtgattttgttggttgcatccatgatattttccaagataacgcagaggctttcccagaaagaacgtgtgtcgtagaaactccaacattgaattctgataaatcaagaagtttcacttatcgtgatatcaataggactagtaatattgttgctcattacttgataaaaaccggtatcaaaagaggtgatgtagtgatgatttactcgtcacgtggtgttgatttaatggtctgtgtgatgggtgttttgaaggctggtgctactttttcagtcatagatccagcatacccaccagcgagacaaactatttacttaggtgttgcgaaaccgcgtgggttaattgttattagagccgctggccagttagatcagttagttgaagattatataaatgacgaattggaaatagtctccaggattaattccattgcaattcaggaaaatggtaccattgaaggtggtaaattggacaatggtgaagatgttttagctccatatgatcactacaaagatacacgcactggcgttgttgtaggtcctgattccaacccaacattatctttcacaagtggctctgaaggtatcccaaaaggtgttttaggaaggcatttttccttggcgtactattttaattggatgtcgaagagatttaacttgacagagaatgataagttcacaatgctttctggcatagctcacgatcccattcaaagagacatgtttacccccctattcctcggtgcacaattgtacgttccaactcaagatgatataggaacaccaggaagattggccgagtggatgagcaaatatggttgcacggttacccacttgacccccgcaatgggtcaattattgactgctcaagccaccacaccatttccaaagttacaccatgcattttttgttggagatatattaactaagagagactgtttgagacttcaaacattagccgagaattgcagaattgtaaacatgtatggcacaacagagacccaacgtgcggtctcctattttgaggttaaaagcaagaatgacgatcctaattttttgaagaaattgaaggatgtcatgcctgctggaaagggaatgctaaatgttcaattattggttgtgaatcgtaacgaccgtacacaaatatgtggtattggtgaaatcggtgaaatttacgttcgtgctggcggtttagcagaaggttacagaggcctccccgaacttaataaagagaaatttgttaacaattggtttgtggaaaaagatcactggaattatctggataaggataatggtgagccttggagacaattctggctgggcccaagagatcgtctatacaggacgggggatttagggagatatttacctaatggtgattgcgagtgttgtggtagagcagatgatcaagtaaagattagaggatttcgaattgaacttggcgaaatcgatacacacatcagccagcatccattagtcagagagaacatcactttggttcgtaaaaacgctgacaatgaaccaactttgattacatttatggttccaagatttgataagccagatgacttaagcaagttccaatctgatgttcctaaggaagtggaaacagaccctattgttaaaggactgatagggtatcatttgctttctaaggatattcgtaccttcttgaaaaaaaggttggcatcgtatgccatgccctctttaattgtggtcatggataagttaccattgaaccctaatggaaaggtggataagccaaagttacaatttcctactccaaaacagttgaatcttgtagccgagaatacagtttctgaaacagatgattcccaatttactaatgttgaaagggaagttcgtgatttatggttatctattctaccaacaaagcctgccagcgtaagccctgatgactctttcttcgacttaggtggacatagcatcttggctacgaaaatgatttttaccttaaagaagaaactacaagtagatctgcctctgggcacaattttcaaataccctaccatcaaggcatttgcggccgaaatcgatagaattaaatcttccggtggttcatcccaaggtgaagtcgtagaaaatgttactgcaaattatgctgaagatgcaaagaaattagttgaaactttaccatcatcatatccttctcgtgaatacttcgtcgagccaaattcagcagaaggtaaaaccactattaatgtgtttgtgactggcgttactggatttttaggttcttatatcctggccgatttgttaggtagatcaccaaaaaattattctttcaaagtatttgctcacgttagagcgaaagatgaggaagctgcatttgccaggttgcagaaggctggtatcacctatggtacttggaacgaaaagtttgcatctaatatcaaagttgttctaggagatttgagcaagtctcagttcggcttaagtgatgaaaaatggatggatttggccaatacggttgatattatcattcataatggagctttggtccactgggtttacccatatgccaaattgagagacccaaatgttattagcacgatcaacgttatgtcattagctgccgttggtaagccaaaattttttgattttgtctcttctacttccacattagataccgagtattattttaatttgagcgacaaattagtttctgaaggtaagcctggtattcttgaatcagacgacttgatgaatagtgcaagtggtttaactggcggttatggtcaatctaaatgggcagccgagtatataatcaggagggccggtgaacgcggtttacgtggctgcattgttcgtccgggctatgttactggtgcatctgcgaatggttcttcaaatacagatgatttcctattacgtttcttaaagggatctgttcaattgggtaagatcccagatatagaaaatagtgttaacatggttcctgtggaccatgttgccagagttgttgttgcaacgagcttgaatccacctaaagagaacgaattggccgttgctcaagtaactggtcaccctcgtatcctatttaaagactatttgtacactttgcatgattatggttatgatgtcgagatagaatcatattccaaatggaaaaaatccttggaagcgtctgtcattgatagaaatgaagagaacgcattgtacccactattacacatggttttagacaacttacctgaatccacaaaggcaccagaattagatgatagaaacgctgttgcctccctaaaaaaggacacagcgtggactggtgtcgattggtctaatggtatcggtgttactccggaggaagttggcatctacattgcctttttaaacaaggttggatttttacctccaccaactcataatgacaaacttccactgccaagtatagaactaactcaagcgcaaataagtctagttgcttcaggtgctggtgctcgtggaagctccgcagcagcttaa

SEQ ID NO: 17 - DNA, 4179 bp, Artificial Sequence
  Lys2FIF1
atgactaacgaaaaggtctggatagagaagttggataatccaactctttcagtgttaccacatgactttttacgcccacaacaagaaccttatacgaaacaagctacatattcgttacagctacctcagctagatgtcccacatgattcattttctaataagtatgcggttgccttatcagtgtgggcggccttaatttaccgcgtcaccggtgacgatgatattgttttgtacatagctaataataaaattttgagattcaatattcaacctacttggtctttcaacgaattgtactccaccattaataatgaattgaataaacttaatagcattgaagctaatttctcctttgacgaattagcagagaaaatacaaagctgtcaagatttagaaagaactcctcagttgtttagattagctttcttagaaaatcaagatttcaagttagatgaatttaagcaccatttggtagatttcgcattaaatttagatacctctaataatgcgcacgtcttaaacttaatttataactcgctactttattctaacgaaagggtaaccatcgtcgcagatcaatttactcaataccttactgctgcgctttctgatccaagtaactgcatcacaaaaatttcactgattaccgcttcttctaaggattccttaccagacccaacaaagaatcttggctggtgtgattttgttggctgtatccacgatatttttcaagataatgccgaggcttttcctgaaaggacctgtgtagtcgaaactccaacattaaactcggacaaatcacgcagtttcacctatcgtgacatcaatcgtacatctaatatcgttgcacactacttaataaagactggaatcaaacgaggtgatgttgtgatgatatattcttcacgtggtgtggatttaatggtgtgtgtcatgggagttctaaaagcaggtgcaacgttttcagtcattgatcccgcctaccctccagctagacaaacaatttacttaggtgttgcgaaaccgcgtggtttgatcgtcattcgtgccgctggacaactagaccaattggttgaagattacattaatgacgaattagaaattgttagtaggattaatagtattgcaatccaagaaaatggaaccattgaaggtggaaaattggataatggtgaagacgtcttagccccatacgaccattataaagatacaagaacaggagtcgtggttggtcctgactcaaatccaacattgtcctttacttctggatctgaaggtataccgaaaggcgttttgggtaggcatttttccctagcatattattttaattggatgtccaaacgcttcaacttaactgaaaatgacaagttcacaatgttatcaggtatagcacatgatccaatacagagagacatgttcactcctttgttcttaggtgctcaactatatgtcccaacacaagatgatatcggcacaccaggtcgactggccgaatggatgtccaaatatggttgcacagtgacacacttaacaccagctatgggtcagttgttaaccgctcaagcgaccacaccttttccaaagttgcaccatgccttcttcgtaggtgatatacttacaaagagggattgtttacgtctgcagacattggcagaaaactgtaggattgtgaacatgtatggtactacagaaacccaaagggccgtgagttattttgaggttaagagtaagaatgatgacccaaatttcttgaaaaaattaaaagacgtcatgccagccggtaaaggtatgttgaacgttcaattgttggttgtaaatagaaacgacaggacccaaatttgtggcatcggtgaaatcggtgaaatctatgtaagagctggtggcttggcagaagggtatcgcggactacctgaattgaacaaggagaaatttgtgaataattggtttgtcgagaaggatcactggaactacttagacaaggataatggtgaaccctggagacaattctggctgggcccaagagatagattgtatcgtactggtgacttaggccgctatttaccaaacggtgattgcgagtgttgtggtcgtgccgatgatcaagttaaaatcagaggttttcgtattgaattaggagaaatcgatacccatataagtcagcaccctttagttagggaaaatattactttggttagaaagaacgcggataacgagccaacattaatcacgttcatggttccaaggtttgataaaccggacgatttgtcaaaattccaatctgatgttccaaaagaagtagagactgatccaattgtaaaaggtttaatagggtaccacctcttgtccaaagatattagaacttttcttaagaagcgtttagcttcttatgctatgccttccctgatcgtggttatggataaattgcctttaaatccaaatggtaaagttgacaagccaaagctgcaattcccaacacctaagcaactaaacttggttgccgagaacacggtttctgagactgacgatagccagttcacaaacgttgaaagggaagtaagggacctttggttgtcaatcttgccaaccaaaccagcctcagttagcccagatgactcattttttgatctaggtggtcattctatcttagcaacgaaaatgatttttactttaaagaaaaagttgcaagttgacttacctcttggtactattttcaaatacccaaccatcaaggcgttcgcggctgagatagacagaataaaatctagcggtggctcctctcaaggtgaagttgtggaaaatgttacagcaaactatgccgaggatgccaaaaaattagttgagacgttaccatcttcctacccatctcgtgaatattttgttgagccaaattcagcggaggggaaaactacaattaatgtgttcgttactggagtcacaggtttcttaggctcatatatcttagcagatttgttaggtcgttcaccaaagaactattcattcaaagtttttgcacatgttagagctaaggatgaagaggccgcatttgcaaggttgcaaaaagccgggatcacctacggtacctggaatgaaaagttcgctagcaacataaaggttgttttgggcgatttgtctaaatctcagtttgggttaagtgacgaaaaatggatggacttagcaaatactgtggatattatcatacacaacggtgccttagttcattgggtctatccatatgccaagttacgtgatcccaatgtaatttctactattaatgtcatgagcctagcggcggttggcaaaccgaaattttttgattttgtttctagtacatcaactttagacacggaatattactttaacttaagtgataagttggttagcgaaggtaaaccaggtatcttggaatctgacgatttgatgaattctgcttctggattgactggtggttacggtcaaagtaaatgggccgctgaatacataattagacgtgcaggtgaacgtggtttacgtggttgcattgtccgtcctggttatgttaccggtgcctctgcgaatggcagctccaacactgacgatttcttgttgagattcttgaaaggatctgtccagctaggtaaaattcctgacattgagaattcggttaatatggttcctgttgatcacgtggccagggttgtcgttgcaacaagcttgaatccacctaaagaaaatgaattagctgttgcgcaagttacggggcatccaaggattttgtttaaagattatttatatacattgcatgactatggttatgacgttgaaatagaatcttactccaaatggaaaaaatctctcgaagctagcgtcatcgatagaaacgaagaaaatgccttataccctttgttacacatggtcctagataacttaccagaatccaccaaagcaccagaattagacgatagaaatgctgttgcatcattaaagaaggataccgcttggactggagttgattggagcaatggtattggggtaacaccagaagaagttggtatttatatcgcatttttgaataaagttggatttttacctccaccaactcataatgacaaacttccactgccaagtatagaactaactcaagcgcaaataagtctagttgcttcaggtgctggtgctcgtggaagctccgcagcagcttaa

SEQ ID NO: 18 - DNA, 4179 bp, Artificial Sequence
  Lys2FIF23
atgactaacgaaaaggtctggatagagaagttggataatccaactctttcagtgttaccacatgactttttacgcccacaacaagaaccttatacgaaacaagctacatattcgttacagctacctcagctcgatgtaccccacgattcattctcaaacaaatatgcggtcgctttaagtgtttgggccgctttaatttacagagttaccggcgatgatgacattgtgttgtatatcgccaataataaaatattaagattcaatattcagccaacatggtctttcaatgaattgtattccacgattaacaacgagttgaataagttaaactccattgaagcgaatttttcgttcgatgagctagctgagaagatccaaagctgtcaagacttggaaaggacacctcaattatttcgcttggcgtttttggaaaatcaagattttaagctagatgaatttaaacatcatttagttgattttgccttgaatctagacaccagtaacaatgctcatgttttaaatttgatttacaactctctgctatattccaacgagcgtgttactatcgtggcggaccaatttactcagtatctgaccgcagccttgtccgatccatctaattgcattaccaaaatttctctaatcactgctagttccaaggatagtttacctgatccgacaaaaaatttgggctggtgtgactttgtcggttgtattcacgatatattccaagacaatgctgaggcgttcccagaacgtacttgcgtcgtagaaacacctaccttaaattctgacaaatctcgttcattcacttaccgtgatatcaacagaacctctaatatcgtggcacattatttaatcaaaactggtatcaagaggggcgacgttgttatgatttattcttccaggggcgtagatttgatggtttgtgtcatgggtgtgttgaaagcaggtgccacgttctccgtaattgatccagcttacccaccagctcgtcaaacaatatatttgggtgttgccaagccaaggggcctaattgttattcgtgctgctggtcaacttgatcaattagttgaagattatattaacgatgaacttgaaattgttagtagaattaattctattgccattcaagaaaacggtacaattgaaggcggtaaattagacaatggtgaagatgttttagccccttacgatcactacaaagatactagaacaggtgttgttgttggtcctgattctaaccctacattatctttcacatcaggttctgaaggtattcctaagggtgttttgggtcgtcatttttcgttggcatactatttcaattggatgtcaaagagattcaatttaactgaaaatgacaagttcacgatgttgagtgggattgctcacgaccctattcaaagagatatgttcactccattattcttgggcgctcaactctatgttcccactcaagatgatatcggtacgcctggtcgtttagctgagtggatgtccaaatacggttgcaccgtaacacacttgacgccagccatgggacagttgttaacagctcaagccaccaccccattcccaaagttacatcacgcctttttcgtcggtgatattttaacaaagcgtgattgtttaaggcttcaaacattggcagaaaattgcagaattgttaatatgtatggtactaccgaaacacaaagagccgtttcatattttgaagttaagagcaagaacgatgaccctaactttctgaagaagttgaaggacgtgatgccagcagggaagggaatgttgaatgttcaattattagttgttaatagaaatgatagaactcaaatttgtggtattggtgagataggtgaaatttatgttcgtgcaggtgggttagctgaaggctacagagggttaccagagttaaataaagagaaatttgtcaataattggttcgtggaaaaagaccattggaattatcttgacaaggataacggtgaaccatggcgccaattttggttgggtcctagggacagattgtacagaacaggtgatctgggtagatatcttcctaatggtgactgcgaatgctgtggtcgtgcagatgatcaagtgaaaattagaggttttcgcattgaattgggagaaatcgatactcacatctcgcaacatccattggtaagggaaaatattacgctagtaagaaaaaatgctgacaatgaacccacattgattacttttatggtgccgaggttcgataaacctgatgacctgtctaaatttcagtcagatgtaccaaaggaagttgaaactgaccctattgttaaaggtcttattgggtaccatttgttatcgaaggacattaggacattcttgaagaagcgcctagcctcctacgccatgccaagtttgattgttgtcatggataaactaccattaaatccaaatggtaaagttgataagccaaaattacaatttccaacacctaagcagttaaatttagttgctgaaaataccgtttcagagaccgatgattctcaattcactaacgttgagagagaagttagagatttgtggttatcaatattgccaactaagccagcgtctgtttcaccagatgattccttttttgatttaggtggtcactccattttggccaccaaaatgatattcactttaaaaaagaagttgcaagttgatcttcctttagggacaattttcaaatatcccacgattaaagccttcgccgcggaaatcgatcgtattaaatcctcaggtggttcgtcccaaggtgaagtagttgaaaacgttactgctaattatgcggaagacgctaagaaacttgtcgaaacattgccaagttcctatccttctagggaatattttgttgaaccaaattccgctgagggtaaaaccaccatcaacgttttcgtcaccggtgttactggttttttgggttcttatattttagctgatttattaggtcgctcccctaaaaattattcatttaaagtttttgctcatgtaagagctaaggatgaagaggctgcctttgcccgtttgcaaaaagcaggtattacatatggtacttggaacgaaaaattcgccagcaatatcaaggtagttttgggtgatttatctaaatctcaatttggtttgagcgacgaaaaatggatggatttagctaacacagtggacattattatccacaatggtgcattggtacactgggtttatccatatgcaaaattgagggatccaaatgttatcagcactataaacgttatgtcactcgccgccgttggaaaacctaaattttttgactttgtctcatctacctctactctagacacagaatattatttcaatctctctgacaagttagtatcagaaggaaaaccagggatactagaatctgatgatttaatgaactccgcaagtggtttgactggtggctatggacaaagtaagtgggcagcggaatacattataagaagagcgggtgaaagaggcttgagaggttgtattgttcgtccaggatacgtcacaggtgcatctgcgaacggctcctcaaataccgatgactttttgttgagatttttaaaaggttctgttcaattgggtaaaattccagatattgagaatagcgtaaatatggtaccagttgaccatgttgcaagagttgtcgttgccacgtctttaaatccaccaaaggaaaatgaattggctgtagcacaggtgacaggtcatccaagaattttattcaaagattatttatataccttgcatgactatggttacgacgtggagatcgaatcctattctaagtggaagaaatctcttgaagcatctgttatcgaccgtaacgaggaaaatgccctgtatccattattacacatggttttagataatttgccagaaagcactaaggccccagagttggatgacaggaacgccgtcgcctctctaaagaaggacacagcttggactggtgttgactggagcaacggtataggtgttaccccagaagaagttggaatatacattgcattcttgaataaggttggatttttacctccaccaactcataatgacaaacttccactgccaagtatagaactaactcaagcgcaaataagtctagttgcttcaggtgctggtgctcgtggaagctccgcagcagcttaa

SEQ ID NO: 19 - DNA, 6 bp, Artificial Sequence
  Rare Frame Dependent Hexamer (averaged)
tctagc

SEQ ID NO: 20 - DNA, 6 bp, Artificial Sequence
  Rare Frame Dependent Hexamer (averaged)
gctatg

SEQ ID NO: 21 - DNA, 6 bp, Artificial Sequence
  Rare Frame Dependent Hexamer (averaged)
gctaag

SEQ ID NO: 22 - DNA, 6 bp, Artificial Sequence
  Rare Frame Dependent Hexamer (averaged)
ctcgct

SEQ ID NO: 23 - DNA, 6 bp, Artificial Sequence
  Rare Frame Dependent Hexamer (averaged)
ttcgct

SEQ ID NO: 24 - DNA, 6 bp, Artificial Sequence
  Rare Frame Dependent Hexamer (averaged)
ctcgca

SEQ ID NO: 25 - DNA, 6 bp, Artificial Sequence
  Rare Frame Dependent Hexamer (averaged)
ttcgca

SEQ ID NO: 26 - DNA, 6 bp, Artificial Sequence
  Rare Frame Dependent Hexamer (averaged)
tgcgct

SEQ ID NO: 27 - DNA, 6 bp, Artificial Sequence
  Rare Frame Dependent Hexamer (averaged)
ctccca

SEQ ID NO: 28 - DNA, 6 bp, Artificial Sequence
  Rare Frame Dependent Hexamer (averaged)
gctaac

SEQ ID NO: 29 - DNA, 6 bp, Artificial Sequence
  Rare Frame Dependent Hexamer (averaged)
tgtagc

SEQ ID NO: 30 - DNA, 6 bp, Artificial Sequence
  Rare Frame Dependent Hexamer (averaged)
gctagc

SEQ ID NO: 31 - DNA, 6 bp, Artificial Sequence
  Rare Frame Dependent Hexamer (averaged)
tttagg

SEQ ID NO: 32 - DNA, 6 bp, Artificial Sequence
  Rare Frame Dependent Hexamer (averaged)
ctcgtg

SEQ ID NO: 33 - DNA, 6 bp, Artificial Sequence
  Rare Frame Dependent Hexamer (averaged)
tgtaga

SEQ ID NO: 34 - DNA, 6 bp, Artificial Sequence
  Rare Frame Dependent Hexamer (averaged)
tccgct

SEQ ID NO: 35 - DNA, 6 bp, Artificial Sequence
  Rare Frame Dependent Hexamer (averaged)
ctcctg

SEQ ID NO: 36 - DNA, 6 bp, Artificial Sequence
  Rare Frame Dependent Hexamer (averaged)
gctaca

SEQ ID NO: 37 - DNA, 6 bp, Artificial Sequence
  Rare Frame Dependent Hexamer (averaged)
ctccaa

SEQ ID NO: 38 - DNA, 6 bp, Artificial Sequence
  Rare Frame Dependent Hexamer (averaged)
gccgct

SEQ ID NO: 39 - DNA, 6 bp, Artificial Sequence
  Rare Frame Dependent Hexamer (averaged)
attagg

SEQ ID NO: 40 - DNA, 6 bp, Artificial Sequence
  Rare Frame Dependent Hexamer (averaged)
cataga

SEQ ID NO: 41 - DNA, 6 bp, Artificial Sequence
  Rare Frame Dependent Hexamer (averaged)
gtcgct

SEQ ID NO: 42 - DNA, 6 bp, Artificial Sequence
  Rare Frame Dependent Hexamer (averaged)
tgtagg

SEQ ID NO: 43 - DNA, 6 bp, Artificial Sequence
  Rare Frame Dependent Hexamer (averaged)
accgct

SEQ ID NO: 44 - DNA, 6 bp, Artificial Sequence
  Rare Frame Dependent Hexamer (averaged)
catagc

SEQ ID NO: 45 - DNA, 6 bp, Artificial Sequence
  Rare Frame Dependent Hexamer (averaged)
tccgca

SEQ ID NO: 46 - DNA, 6 bp, Artificial Sequence
  Rare Frame Dependent Hexamer (averaged)
gctaaa

SEQ ID NO: 47 - DNA, 6 bp, Artificial Sequence
  Rare Frame Dependent Hexamer (averaged)
ttcgtt

SEQ ID NO: 48 - DNA, 6 bp, Artificial Sequence
  Rare Frame Dependent Hexamer (averaged)
cattgg

SEQ ID NO: 49 - DNA, 6 bp, Artificial Sequence
  Rare Frame Dependent Hexamer (averaged)
gtcgca

SEQ ID NO: 50 - DNA, 6 bp, Artificial Sequence
  Rare Frame Dependent Hexamer (averaged)
gccgca

SEQ ID NO: 51 - DNA, 6 bp, Artificial Sequence
  Rare Frame Dependent Hexamer (averaged)
gctacg

SEQ ID NO: 52 - DNA, 6 bp, Artificial Sequence
  Rare Frame Dependent Hexamer (averaged)
tgcgga

SEQ ID NO: 53 - DNA, 6 bp, Artificial Sequence
  Rare Frame Dependent Hexamer (averaged)
tctaag

SEQ ID NO: 54 - DNA, 6 bp, Artificial Sequence
  Rare Frame Dependent Hexamer (averaged)
atcgct

SEQ ID NO: 55 - DNA, 6 bp, Artificial Sequence
  Rare Frame Dependent Hexamer (averaged)
gatagc

SEQ ID NO: 56 - DNA, 6 bp, Artificial Sequence
  Rare Frame Dependent Hexamer (averaged)
aacgct

SEQ ID NO: 57 - DNA, 6 bp, Artificial Sequence
  Rare Frame Dependent Hexamer (averaged)
caccaa

SEQ ID NO: 58 - DNA, 6 bp, Artificial Sequence
  Rare Frame Dependent Hexamer (averaged)
ttcgga

SEQ ID NO: 59 - DNA, 6 bp, Artificial Sequence
  Rare Frame Dependent Hexamer (averaged)
agtagg

SEQ ID NO: 60 - DNA, 6 bp, Artificial Sequence
  Rare Frame Dependent Hexamer (averaged)
tgcgca

SEQ ID NO: 61 - DNA, 6 bp, Artificial Sequence
  Rare Frame Dependent Hexamer (averaged)
gattgg

SEQ ID NO: 62 - DNA, 6 bp, Artificial Sequence
  Rare Frame Dependent Hexamer (averaged)
actaag

SEQ ID NO: 63 - DNA, 6 bp, Artificial Sequence
  Rare Frame Dependent Hexamer (averaged)
gctacc

SEQ ID NO: 64 - DNA, 6 bp, Artificial Sequence
  Rare Frame Dependent Hexamer (averaged)
accgca

SEQ ID NO: 65 - DNA, 6 bp, Artificial Sequence
  Rare Frame Dependent Hexamer (averaged)
gacgct

SEQ ID NO: 66 - DNA, 6 bp, Artificial Sequence
  Rare Frame Dependent Hexamer (averaged)
actagc

SEQ ID NO: 67 - DNA, 6 bp, Artificial Sequence
  Rare Frame Dependent Hexamer (averaged)
agcgct

SEQ ID NO: 68 - DNA, 6 bp, Artificial Sequence
  Rare Frame Dependent Hexamer (averaged)
aggtgg

SEQ ID NO: 69 - DNA, 6 bp, Artificial Sequence
  Rare Frame Dependent Hexamer (Averaged)
agcgca

SEQ ID NO: 70 - DNA, 6 bp, Artificial Sequence
  Rare Frame Dependent Hexamer (Averaged)
tttagc

SEQ ID NO: 71 - DNA, 6 bp, Artificial Sequence
  Rare Frame Dependent Hexamer (Averaged)
cttaag

SEQ ID NO: 72 - DNA, 6 bp, Artificial Sequence
  Rare Frame Dependent Hexamer (Averaged)
gctagt

SEQ ID NO: 73 - DNA, 6 bp, Artificial Sequence
  Rare Frame Dependent Hexamer (Averaged)
cacgct

SEQ ID NO: 74 - DNA, 6 bp, Artificial Sequence
  Rare Frame Dependent Hexamer (Averaged)
ctcgaa

SEQ ID NO: 75 - DNA, 6 bp, Artificial Sequence
  Rare Frame Dependent Hexamer (Averaged)
ggcgct

SEQ ID NO: 76 - DNA, 6 bp, Artificial Sequence
  Rare Frame Dependent Hexamer (Averaged)
gtcgtg

SEQ ID NO: 77 - DNA, 6 bp, Artificial Sequence
  Rare Frame Dependent Hexamer (Averaged)
cggtgg

SEQ ID NO: 78 - DNA, 6 bp, Artificial Sequence
  Rare Frame Dependent Hexamer (Averaged)
actatg

SEQ ID NO: 79 - DNA, 6 bp, Artificial Sequence
  Rare Frame Dependent Hexamer (Averaged)
gctaat

SEQ ID NO: 80 - DNA, 6 bp, Artificial Sequence
  Rare Frame Dependent Hexamer (Averaged)
gctatc

SEQ ID NO: 81 - DNA, 6 bp, Artificial Sequence
  Rare Frame Dependent Hexamer (Averaged)
tttaag

SEQ ID NO: 82 - DNA, 6 bp, Artificial Sequence
  Rare Frame Dependent Hexamer (Averaged)
cttatg

SEQ ID NO: 83 - DNA, 6 bp, Artificial Sequence
  Rare Frame Dependent Hexamer (Averaged)
agtaga

SEQ ID NO: 84 - DNA, 6 bp, Artificial Sequence
  Rare Frame Dependent Hexamer (Averaged)
ttcgtg

SEQ ID NO: 85 - DNA, 6 bp, Artificial Sequence
  Rare Frame Dependent Hexamer (Averaged)
ttcgaa

SEQ ID NO: 86 - DNA, 6 bp, Artificial Sequence
  Rare Frame Dependent Hexamer (Averaged)
attaga

SEQ ID NO: 87 - DNA, 6 bp, Artificial Sequence
  Rare Frame Dependent Hexamer (Averaged)
tttaga

SEQ ID NO: 88 - DNA, 6 bp, Artificial Sequence
  Rare Frame Dependent Hexamer (Averaged)
aatagg

SEQ ID NO: 89 - DNA, 6 bp, Artificial Sequence
  Rare Frame Dependent Hexamer (Averaged)
ggtaag

SEQ ID NO: 90 - DNA, 6 bp, Artificial Sequence
  Rare Frame Dependent Hexamer (Averaged)
catagg

SEQ ID NO: 91 - DNA, 6 bp, Artificial Sequence
  Rare Frame Dependent Hexamer (Averaged)
atcgca

SEQ ID NO: 92 - DNA, 6 bp, Artificial Sequence
  Rare Frame Dependent Hexamer (Averaged)
gacgca

SEQ ID NO: 93 - DNA, 6 bp, Artificial Sequence
  Rare Frame Dependent Hexamer (Averaged)
tctaga

SEQ ID NO: 94 - DNA, 6 bp, Artificial Sequence
  Rare Frame Dependent Hexamer (Averaged)
ctgcca

SEQ ID NO: 95 - DNA, 6 bp, Artificial Sequence
  Rare Frame Dependent Hexamer (Averaged)
catagt

SEQ ID NO: 96 - DNA, 6 bp, Artificial Sequence
  Rare Frame Dependent Hexamer (Averaged)
tttaac

SEQ ID NO: 97 - DNA, 6 bp, Artificial Sequence
  Rare Frame Dependent Hexamer (Averaged)
gttagg

SEQ ID NO: 98 - DNA, 6 bp, Artificial Sequence
  Rare Frame Dependent Hexamer (Averaged)
tctaac

SEQ ID NO: 99 - DNA, 6 bp, Artificial Sequence
  Rare Frame Dependent Hexamer (Averaged)
attagc

SEQ ID NO: 100 - DNA, 6 bp, Artificial Sequence
  Rare Frame Dependent Hexamer (Averaged)
gggtgg

SEQ ID NO: 101 - DNA, 6 bp, Artificial Sequence
  Rare Frame Dependent Hexamer (Averaged)
ctcgtt

SEQ ID NO: 102 - DNA, 6 bp, Artificial Sequence
  Rare Frame Dependent Hexamer (Averaged)
tgcgtt

SEQ ID NO: 103 - DNA, 6 bp, Artificial Sequence
  Rare Frame Dependent Hexamer (Averaged)
ctcctc

SEQ ID NO: 104 - DNA, 6 bp, Artificial Sequence
  Rare Frame Dependent Hexamer (Averaged)
gctagg

SEQ ID NO: 105 - DNA, 6 bp, Artificial Sequence
  Rare Frame Dependent Hexamer (Averaged)
attaac

SEQ ID NO: 106 - DNA, 6 bp, Artificial Sequence
  Rare Frame Dependent Hexamer (Averaged)
agtaca

SEQ ID NO: 107 - DNA, 6 bp, Artificial Sequence
  Rare Frame Dependent Hexamer (Averaged)
ctccag

SEQ ID NO: 108 - DNA, 6 bp, Artificial Sequence
  Rare Frame Dependent Hexamer (Averaged)
tttagt

SEQ ID NO: 109 - DNA, 6 bp, Artificial Sequence
  Rare Frame Dependent Hexamer (Averaged)
gttatg

SEQ ID NO: 110 - DNA, 6 bp, Artificial Sequence
  Rare Frame Dependent Hexamer (Averaged)
tacgct

SEQ ID NO: 111 - DNA, 6 bp, Artificial Sequence
  Rare Frame Dependent Hexamer (Averaged)
gccgtg

SEQ ID NO: 112 - DNA, 6 bp, Artificial Sequence
  Rare Frame Dependent Hexamer (Averaged)
tgtagt

SEQ ID NO: 113 - DNA, 6 bp, Artificial Sequence
  Rare Frame Dependent Hexamer (Averaged)
ctgtca

SEQ ID NO: 114 - DNA, 6 bp, Artificial Sequence
  Rare Frame Dependent Hexamer (Averaged)
ttttgg

SEQ ID NO: 115 - DNA, 6 bp, Artificial Sequence
  Rare Frame Dependent Hexamer (Averaged)
caccca

SEQ ID NO: 116 - DNA, 6 bp, Artificial Sequence
  Rare Frame Dependent Hexamer (Averaged)
gtcgaa

SEQ ID NO: 117 - DNA, 6 bp, Artificial Sequence
  Rare Frame Dependent Hexamer (Averaged)
cacctg

SEQ ID NO: 118 - DNA, 6 bp, Artificial Sequence
  Rare Frame Dependent Hexamer (Averaged)
cacctg

SEQ ID NO: 119 - DNA, 6 bp, Artificial Sequence
  Rare Frame Independent Hexamer (Averaged)
cccccc

SEQ ID NO: 120 - DNA, 6 bp, Artificial Sequence
  Rare Frame Independent Hexamer (Averaged)
gggggg

SEQ ID NO: 121 - DNA, 6 bp, Artificial Sequence
  Rare Frame Independent Hexamer (Averaged)
accccc

SEQ ID NO: 122 - DNA, 6 bp, Artificial Sequence
  Rare Frame Independent Hexamer (Averaged)
gggggt

SEQ ID NO: 123 - DNA, 6 bp, Artificial Sequence
  Rare Frame Independent Hexamer (Averaged)
cccccg

SEQ ID NO: 124 - DNA, 6 bp, Artificial Sequence
  Rare Frame Independent Hexamer (Averaged)
cggggg

SEQ ID NO: 125 - DNA, 6 bp, Artificial Sequence
  Rare Frame Independent Hexamer (Averaged)
ccccct

SEQ ID NO: 126 - DNA, 6 bp, Artificial Sequence
  Rare Frame Independent Hexamer (Averaged)
gccccc

SEQ ID NO: 127 - DNA, 6 bp, Artificial Sequence
  Rare Frame Independent Hexamer (Averaged)
ccccta

SEQ ID NO: 128 - DNA, 6 bp, Artificial Sequence
  Rare Frame Independent Hexamer (Averaged)
cgcgcg

SEQ ID NO: 129 - DNA, 6 bp, Artificial Sequence
  Rare Frame Independent Hexamer (Averaged)
cgcccc

SEQ ID NO: 130 - DNA, 6 bp, Artificial Sequence
  Rare Frame Independent Hexamer (Averaged)
gcgcga

SEQ ID NO: 131 - DNA, 6 bp, Artificial Sequence
  Rare Frame Independent Hexamer (Averaged)
cgcgaa

SEQ ID NO: 132 - DNA, 6 bp, Artificial Sequence
  Rare Frame Independent Hexamer (Averaged)
tacgta

SEQ ID NO: 133 - DNA, 6 bp, Artificial Sequence
  Rare Frame Independent Hexamer (Averaged)
aggggg

SEQ ID NO: 134 - DNA, 6 bp, Artificial Sequence
  Rare Frame Independent Hexamer (Averaged)
tcgcga

SEQ ID NO: 135 - DNA, 6 bp, Artificial Sequence
  Rare Frame Independent Hexamer (Averaged)
cgcgta

SEQ ID NO: 136 - DNA, 6 bp, Artificial Sequence
  Rare Frame Independent Hexamer (Averaged)
gcgcgc

SEQ ID NO: 137 - DNA, 6 bp, Artificial Sequence
  Rare Frame Independent Hexamer (Averaged)
ccccgc

SEQ ID NO: 138 - DNA, 6 bp, Artificial Sequence
  Rare Frame Independent Hexamer (Averaged)
ggggta

SEQ ID NO: 139 - DNA, 6 bp, Artificial Sequence
  Rare Frame Independent Hexamer (Averaged)
tttttt

SEQ ID NO: 140 - DNA, 6 bp, Artificial Sequence
  Rare Frame Independent Hexamer (Averaged)
ggggtg

SEQ ID NO: 141 - DNA, 6 bp, Artificial Sequence
  Rare Frame Independent Hexamer (Averaged)
aaaaaa

SEQ ID NO: 142 - DNA, 6 bp, Artificial Sequence
  Rare Frame Independent Hexamer (Averaged)
cggtcc

SEQ ID NO: 143 - DNA, 6 bp, Artificial Sequence
  Rare Frame Independent Hexamer (Averaged)
acgcga

SEQ ID NO: 144 - DNA, 6 bp, Artificial Sequence
  Rare Frame Independent Hexamer (Averaged)
ggtccc

SEQ ID NO: 145 - DNA, 6 bp, Artificial Sequence
  Rare Frame Independent Hexamer (Averaged)
cgcgag

SEQ ID NO: 146 - DNA, 6 bp, Artificial Sequence
  Rare Frame Independent Hexamer (Averaged)
cgcgac

SEQ ID NO: 147 - DNA, 6 bp, Artificial Sequence
  Rare Frame Independent Hexamer (Averaged)
tacccc

SEQ ID NO: 148 - DNA, 6 bp, Artificial Sequence
  Rare Frame Independent Hexamer (Averaged)
ttcgcg

SEQ ID NO: 149 - DNA, 6 bp, Artificial Sequence
  Rare Frame Independent Hexamer (Averaged)
cgcgat

SEQ ID NO: 150 - DNA, 6 bp, Artificial Sequence
  Rare Frame Independent Hexamer (Averaged)
cacccc

SEQ ID NO: 151 - DNA, 6 bp, Artificial Sequence
  Rare Frame Independent Hexamer (Averaged)
gtcccc

SEQ ID NO: 152 - DNA, 6 bp, Artificial Sequence
  Rare Frame Independent Hexamer (Averaged)
gggccc

SEQ ID NO: 153 - DNA, 6 bp, Artificial Sequence
  Rare Frame Independent Hexamer (Averaged)
ccccga

SEQ ID NO: 154 - DNA, 6 bp, Artificial Sequence
  Rare Frame Independent Hexamer (Averaged)
gacccc

SEQ ID NO: 155 - DNA, 6 bp, Artificial Sequence
  Rare Frame Independent Hexamer (Averaged)
agcgcg

SEQ ID NO: 156 - DNA, 6 bp, Artificial Sequence
  Rare Frame Independent Hexamer (Averaged)
tccccc

SEQ ID NO: 157 - DNA, 6 bp, Artificial Sequence
  Rare Frame Independent Hexamer (Averaged)
cccgcg

SEQ ID NO: 158 - DNA, 6 bp, Artificial Sequence
  Rare Frame Independent Hexamer (Averaged)
ggcgcc

SEQ ID NO: 159 - DNA, 6 bp, Artificial Sequence
  Rare Frame Independent Hexamer (Averaged)
accccg

SEQ ID NO: 160 - DNA, 6 bp, Artificial Sequence
  Rare Frame Independent Hexamer (Averaged)
gtccta

SEQ ID NO: 161 - DNA, 6 bp, Artificial Sequence
  Rare Frame Independent Hexamer (Averaged)
tacgcg

SEQ ID NO: 162 - DNA, 6 bp, Artificial Sequence
  Rare Frame Independent Hexamer (Averaged)
gtcgcg

SEQ ID NO: 163 - DNA, 6 bp, Artificial Sequence
  Rare Frame Independent Hexamer (Averaged)
gcgccc

SEQ ID NO: 164 - DNA, 6 bp, Artificial Sequence
  Rare Frame Independent Hexamer (Averaged)
ggcccc

SEQ ID NO: 165 - DNA, 6 bp, Artificial Sequence
  Rare Frame Independent Hexamer (Averaged)
cgtacg

SEQ ID NO: 166 - DNA, 6 bp, Artificial Sequence
  Rare Frame Independent Hexamer (Averaged)
ccctat

SEQ ID NO: 167 - DNA, 6 bp, Artificial Sequence
  Rare Frame Independent Hexamer (Averaged)
cgcgca

SEQ ID NO: 168 - DNA, 6 bp, Artificial Sequence
  Rare Frame Independent Hexamer (Averaged)
ggggga

SEQ ID NO: 169 - DNA, 6 bp, Artificial Sequence
  Rare Frame Independent Hexamer (Averaged)
gacgta

SEQ ID NO: 170 - DNA, 6 bp, Artificial Sequence
  Rare Frame Independent Hexamer (Averaged)
cgaacg

SEQ ID NO: 171 - DNA, 6 bp, Artificial Sequence
  Rare Frame Independent Hexamer (Averaged)
ccccca

SEQ ID NO: 172 - DNA, 6 bp, Artificial Sequence
  Rare Frame Independent Hexamer (Averaged)
gtacgt

SEQ ID NO: 173 - DNA, 6 bp, Artificial Sequence
  Rare Frame Independent Hexamer (Averaged)
cggggt

SEQ ID NO: 174 - DNA, 6 bp, Artificial Sequence
  Rare Frame Independent Hexamer (Averaged)
tcgcgc

SEQ ID NO: 175 - DNA, 6 bp, Artificial Sequence
  Rare Frame Independent Hexamer (Averaged)
ctcgcg

SEQ ID NO: 176 - DNA, 6 bp, Artificial Sequence
  Rare Frame Independent Hexamer (Averaged)
ttcgta

SEQ ID NO: 177 - DNA, 6 bp, Artificial Sequence
  Rare Frame Independent Hexamer (Averaged)
gaggta

SEQ ID NO: 178 - DNA, 6 bp, Artificial Sequence
  Rare Frame Independent Hexamer (Averaged)
tcgcgt

SEQ ID NO: 179 - DNA, 6 bp, Artificial Sequence
  Rare Frame Independent Hexamer (Averaged)
ttacgt

SEQ ID NO: 180 - DNA, 6 bp, Artificial Sequence
  Rare Frame Independent Hexamer (Averaged)
cggccg

SEQ ID NO: 181 - DNA, 6 bp, Artificial Sequence
  Rare Frame Independent Hexamer (Averaged)
aacgcg

SEQ ID NO: 182 - DNA, 6 bp, Artificial Sequence
  Rare Frame Independent Hexamer (Averaged)
tatacg

SEQ ID NO: 183 - DNA, 6 bp, Artificial Sequence
  Rare Frame Independent Hexamer (Averaged)
cggtcg

SEQ ID NO: 184 - DNA, 6 bp, Artificial Sequence
  Rare Frame Independent Hexamer (Averaged)
aggtac

SEQ ID NO: 185 - DNA, 6 bp, Artificial Sequence
  Rare Frame Independent Hexamer (Averaged)
aggggt

SEQ ID NO: 186 - DNA, 6 bp, Artificial Sequence
  Rare Frame Independent Hexamer (Averaged)
tgcgcg

SEQ ID NO: 187 - DNA, 6 bp, Artificial Sequence
  Rare Frame Independent Hexamer (Averaged)
gccccg

SEQ ID NO: 188 - DNA, 6 bp, Artificial Sequence
  Rare Frame Independent Hexamer (Averaged)
acccct

SEQ ID NO: 189 - DNA, 6 bp, Artificial Sequence
  Rare Frame Independent Hexamer (Averaged)
cgtgcg

SEQ ID NO: 190 - DNA, 6 bp, Artificial Sequence
  Rare Frame Independent Hexamer (Averaged)
aacgta

SEQ ID NO: 191 - DNA, 6 bp, Artificial Sequence
  Rare Frame Independent Hexamer (Averaged)
ccccgt

SEQ ID NO: 192 - DNA, 6 bp, Artificial Sequence
  Rare Frame Independent Hexamer (Averaged)
gcgcgt

SEQ ID NO: 193 - DNA, 6 bp, Artificial Sequence
  Rare Frame Independent Hexamer (Averaged)
cgtata

SEQ ID NO: 194 - DNA, 6 bp, Artificial Sequence
  Rare Frame Independent Hexamer (Averaged)
gcccct

SEQ ID NO: 195 - DNA, 6 bp, Artificial Sequence
  Rare Frame Independent Hexamer (Averaged)
cttacg

SEQ ID NO: 196 - DNA, 6 bp, Artificial Sequence
  Rare Frame Independent Hexamer (Averaged)
ccgcga

SEQ ID NO: 197 - DNA, 6 bp, Artificial Sequence
  Rare Frame Independent Hexamer (Averaged)
agcccc

SEQ ID NO: 198 - DNA, 6 bp, Artificial Sequence
  Rare Frame Independent Hexamer (Averaged)
acgtac

SEQ ID NO: 199 - DNA, 6 bp, Artificial Sequence
  Rare Frame Independent Hexamer (Averaged)
ccgcgg

SEQ ID NO: 200 - DNA, 6 bp, Artificial Sequence
  Rare Frame Independent Hexamer (Averaged)
gcccta

SEQ ID NO: 201 - DNA, 6 bp, Artificial Sequence
  Rare Frame Independent Hexamer (Averaged)
atacgt

SEQ ID NO: 202 - DNA, 6 bp, Artificial Sequence
  Rare Frame Independent Hexamer (Averaged)
gcgggg

SEQ ID NO: 203 - DNA, 6 bp, Artificial Sequence
  Rare Frame Independent Hexamer (Averaged)
gggtcc

SEQ ID NO: 204 - DNA, 6 bp, Artificial Sequence
  Rare Frame Independent Hexamer (Averaged)
ccccgg

SEQ ID NO: 205 - DNA, 6 bp, Artificial Sequence
  Rare Frame Independent Hexamer (Averaged)
cccctc

SEQ ID NO: 206 - DNA, 6 bp, Artificial Sequence
  Rare Frame Independent Hexamer (Averaged)
gtccga

SEQ ID NO: 207 - DNA, 6 bp, Artificial Sequence
  Rare Frame Independent Hexamer (Averaged)
gggggc

SEQ ID NO: 208 - DNA, 6 bp, Artificial Sequence
  Rare Frame Independent Hexamer (Averaged)
cccgta

SEQ ID NO: 209 - DNA, 6 bp, Artificial Sequence
  Rare Frame Independent Hexamer (Averaged)
gttgcg

SEQ ID NO: 210 - DNA, 6 bp, Artificial Sequence
  Rare Frame Independent Hexamer (Averaged)
acgcgc

SEQ ID NO: 211 - DNA, 6 bp, Artificial Sequence
  Rare Frame Independent Hexamer (Averaged)
cgcata

SEQ ID NO: 212 - DNA, 6 bp, Artificial Sequence
  Rare Frame Independent Hexamer (Averaged)
tcgtac

SEQ ID NO: 213 - DNA, 6 bp, Artificial Sequence
  Rare Frame Independent Hexamer (Averaged)
ggggtc

SEQ ID NO: 214 - DNA, 6 bp, Artificial Sequence
  Rare Frame Independent Hexamer (Averaged)
aacccc

SEQ ID NO: 215 - DNA, 6 bp, Artificial Sequence
  Rare Frame Independent Hexamer (Averaged)
gagggg

SEQ ID NO: 216 - DNA, 6 bp, Artificial Sequence
  Rare Frame Independent Hexamer (Averaged)
caggta

SEQ ID NO: 217 - DNA, 6 bp, Artificial Sequence
  Rare Frame Independent Hexamer (Averaged)
gtcgta

SEQ ID NO: 218 - DNA, 6 bp, Artificial Sequence
  Rare Frame Independent Hexamer (Averaged)
acgggg

SEQ ID NO: 219 - DNA, 6 bp, Artificial Sequence
  Rare Frame Dependent Hexamer (H. sapiens)
gccgct

SEQ ID NO: 220 - DNA, 6 bp, Artificial Sequence
  Rare Frame Dependent Hexamer (H. sapiens)
ctcgaa

SEQ ID NO: 221 - DNA, 6 bp, Artificial Sequence
  Rare Frame Dependent Hexamer (H. sapiens)
ctcgct

SEQ ID NO: 222 - DNA, 6 bp, Artificial Sequence
  Rare Frame Dependent Hexamer (H. sapiens)
cccgct

SEQ ID NO: 223 - DNA, 6 bp, Artificial Sequence
  Rare Frame Dependent Hexamer (H. sapiens)
ctcgga

SEQ ID NO: 224 - DNA, 6 bp, Artificial Sequence
  Rare Frame Dependent Hexamer (H. sapiens)
gtcgct

SEQ ID NO: 225 - DNA, 6 bp, Artificial Sequence
  Rare Frame Dependent Hexamer (H. sapiens)
ggcgct

SEQ ID NO: 226 - DNA, 6 bp, Artificial Sequence
  Rare Frame Dependent Hexamer (H. sapiens)
tccgct

SEQ ID NO: 227 - DNA, 6 bp, Artificial Sequence
  Rare Frame Dependent Hexamer (H. sapiens)
accgct

SEQ ID NO: 228 - DNA, 6 bp, Artificial Sequence
  Rare Frame Dependent Hexamer (H. sapiens)
tgcgct

SEQ ID NO: 229 - DNA, 6 bp, Artificial Sequence
  Rare Frame Dependent Hexamer (H. sapiens)
gccgca

SEQ ID NO: 230 - DNA, 6 bp, Artificial Sequence
  Rare Frame Dependent Hexamer (H. sapiens)
ctcgag

SEQ ID NO: 231 - DNA, 6 bp, Artificial Sequence
  Rare Frame Dependent Hexamer (H. sapiens)
cgcgct

SEQ ID NO: 232 - DNA, 6 bp, Artificial Sequence
  Rare Frame Dependent Hexamer (H. sapiens)
ctcgca

SEQ ID NO: 233 - DNA, 6 bp, Artificial Sequence
  Rare Frame Dependent Hexamer (H. sapiens)
gccgga

SEQ ID NO: 234 - DNA, 6 bp, Artificial Sequence
  Rare Frame Dependent Hexamer (H. sapiens)
tgcgga

SEQ ID NO: 235 - DNA, 6 bp, Artificial Sequence
  Rare Frame Dependent Hexamer (H. sapiens)
ttcgaa

SEQ ID NO: 236 - DNA, 6 bp, Artificial Sequence
  Rare Frame Dependent Hexamer (H. sapiens)
ctcggt

SEQ ID NO: 237 - DNA, 6 bp, Artificial Sequence
  Rare Frame Dependent Hexamer (H. sapiens)
gtcgaa

SEQ ID NO: 238 - DNA, 6 bp, Artificial Sequence
  Rare Frame Dependent Hexamer (H. sapiens)
tccgca

SEQ ID NO: 239 - DNA, 6 bp, Artificial Sequence
  Rare Frame Dependent Hexamer (H. sapiens)
gtcgca

SEQ ID NO: 240 - DNA, 6 bp, Artificial Sequence
  Rare Frame Dependent Hexamer (H. sapiens)
agcgct

SEQ ID NO: 241 - DNA, 6 bp, Artificial Sequence
  Rare Frame Dependent Hexamer (H. sapiens)
accgca

SEQ ID NO: 242 - DNA, 6 bp, Artificial Sequence
  Rare Frame Dependent Hexamer (H. sapiens)
gtcgga

SEQ ID NO: 243 - DNA, 6 bp, Artificial Sequence
  Rare Frame Dependent Hexamer (H. sapiens)
gtcgag

SEQ ID NO: 244 - DNA, 6 bp, Artificial Sequence
  Rare Frame Dependent Hexamer (H. sapiens)
ttcgct

SEQ ID NO: 245 - DNA, 6 bp, Artificial Sequence
  Rare Frame Dependent Hexamer (H. sapiens)
ctcggc

SEQ ID NO: 246 - DNA, 6 bp, Artificial Sequence
  Rare Frame Dependent Hexamer (H. sapiens)
ttcgga

SEQ ID NO: 247 - DNA, 6 bp, Artificial Sequence
  Rare Frame Dependent Hexamer (H. sapiens)
cataga

SEQ ID NO: 248 - DNA, 6 bp, Artificial Sequence
  Rare Frame Dependent Hexamer (H. sapiens)
tccgaa

SEQ ID NO: 249 - DNA, 6 bp, Artificial Sequence
  Rare Frame Dependent Hexamer (H. sapiens)
tccgga

SEQ ID NO: 250 - DNA, 6 bp, Artificial Sequence
  Rare Frame Dependent Hexamer (H. sapiens)
tgcgca

SEQ ID NO: 251 - DNA, 6 bp, Artificial Sequence
  Rare Frame Dependent Hexamer (H. sapiens)
ggcgca

SEQ ID NO: 252 - DNA, 6 bp, Artificial Sequence
  Rare Frame Dependent Hexamer (H. sapiens)
cccgga

SEQ ID NO: 253 - DNA, 6 bp, Artificial Sequence
  Rare Frame Dependent Hexamer (H. sapiens)
ggcgga

SEQ ID NO: 254 - DNA, 6 bp, Artificial Sequence
  Rare Frame Dependent Hexamer (H. sapiens)
accgaa

SEQ ID NO: 255 - DNA, 6 bp, Artificial Sequence
  Rare Frame Dependent Hexamer (H. sapiens)
ctcgcc

SEQ ID NO: 256 - DNA, 6 bp, Artificial Sequence
  Rare Frame Dependent Hexamer (H. sapiens)
catagc

SEQ ID NO: 257 - DNA, 6 bp, Artificial Sequence
  Rare Frame Dependent Hexamer (H. sapiens)
ttcgtt

SEQ ID NO: 258 - DNA, 6 bp, Artificial Sequence
  Rare Frame Dependent Hexamer (H. sapiens)
cccgca

SEQ ID NO: 259 - DNA, 6 bp, Artificial Sequence
  Rare Frame Dependent Hexamer (H. sapiens)
cctagc

SEQ ID NO: 260 - DNA, 6 bp, Artificial Sequence
  Rare Frame Dependent Hexamer (H. sapiens)
cacgct

SEQ ID NO: 261 - DNA, 6 bp, Artificial Sequence
  Rare Frame Dependent Hexamer (H. sapiens)
accgga

SEQ ID NO: 262 - DNA, 6 bp, Artificial Sequence
  Rare Frame Dependent Hexamer (H. sapiens)
agcgca

SEQ ID NO: 263 - DNA, 6 bp, Artificial Sequence
  Rare Frame Dependent Hexamer (H. sapiens)
gccgaa

SEQ ID NO: 264 - DNA, 6 bp, Artificial Sequence
  Rare Frame Dependent Hexamer (H. sapiens)
gtcggc

SEQ ID NO: 265 - DNA, 6 bp, Artificial Sequence
  Rare Frame Dependent Hexamer (H. sapiens)
gtcgcc

SEQ ID NO: 266 - DNA, 6 bp, Artificial Sequence
  Rare Frame Dependent Hexamer (H. sapiens)
gctagg

SEQ ID NO: 267 - DNA, 6 bp, Artificial Sequence
  Rare Frame Dependent Hexamer (H. sapiens)
gccgcc

SEQ ID NO: 268 - DNA, 6 bp, Artificial Sequence
  Rare Frame Dependent Hexamer (H. sapiens)
catagg

SEQ ID NO: 269 - DNA, 6 bp, Artificial Sequence
  Rare Frame Dependent Hexamer (H. sapiens)
tccgcc

SEQ ID NO: 270 - DNA, 6 bp, Artificial Sequence
  Rare Frame Dependent Hexamer (H. sapiens)
tccgtt

SEQ ID NO: 271 - DNA, 6 bp, Artificial Sequence
  Rare Frame Dependent Hexamer (H. sapiens)
aacgct

SEQ ID NO: 272 - DNA, 6 bp, Artificial Sequence
  Rare Frame Dependent Hexamer (H. sapiens)
gctagc

SEQ ID NO: 273 - DNA, 6 bp, Artificial Sequence
  Rare Frame Dependent Hexamer (H. sapiens)
cctaga

SEQ ID NO: 274 - DNA, 6 bp, Artificial Sequence
  Rare Frame Dependent Hexamer (H. sapiens)
tgtagc

SEQ ID NO: 275 - DNA, 6 bp, Artificial Sequence
  Rare Frame Dependent Hexamer (H. sapiens)
ctcggg

SEQ ID NO: 276 - DNA, 6 bp, Artificial Sequence
  Rare Frame Dependent Hexamer (H. sapiens)
cccgtt

SEQ ID NO: 277 - DNA, 6 bp, Artificial Sequence
  Rare Frame Dependent Hexamer (H. sapiens)
agcgtt

SEQ ID NO: 278 - DNA, 6 bp, Artificial Sequence
  Rare Frame Dependent Hexamer (H. sapiens)
cccggt

SEQ ID NO: 279 - DNA, 6 bp, Artificial Sequence
  Rare Frame Dependent Hexamer (H. sapiens)
cctagg

SEQ ID NO: 280 - DNA, 6 bp, Artificial Sequence
  Rare Frame Dependent Hexamer (H. sapiens)
agcgga

SEQ ID NO: 281 - DNA, 6 bp, Artificial Sequence
  Rare Frame Dependent Hexamer (H. sapiens)
gccgtt

SEQ ID NO: 282 - DNA, 6 bp, Artificial Sequence
  Rare Frame Dependent Hexamer (H. sapiens)
gacgct

SEQ ID NO: 283 - DNA, 6 bp, Artificial Sequence
  Rare Frame Dependent Hexamer (H. sapiens)
cgcgca

SEQ ID NO: 284 - DNA, 6 bp, Artificial Sequence
  Rare Frame Dependent Hexamer (H. sapiens)
tccgag

SEQ ID NO: 285 - DNA, 6 bp, Artificial Sequence
  Rare Frame Dependent Hexamer (H. sapiens)
ggtagg

SEQ ID NO: 286 - DNA, 6 bp, Artificial Sequence
  Rare Frame Dependent Hexamer (H. sapiens)
gtcgtt

SEQ ID NO: 287 - DNA, 6 bp, Artificial Sequence
  Rare Frame Dependent Hexamer (H. sapiens)
tgtagg

SEQ ID NO: 288 - DNA, 6 bp, Artificial Sequence
  Rare Frame Dependent Hexamer (H. sapiens)
cccgaa

SEQ ID NO: 289 - DNA, 6 bp, Artificial Sequence
  Rare Frame Dependent Hexamer (H. sapiens)
ggcgaa

SEQ ID NO: 290 - DNA, 6 bp, Artificial Sequence
  Rare Frame Dependent Hexamer (H. sapiens)
tgtaga

SEQ ID NO: 291 - DNA, 6 bp, Artificial Sequence
  Rare Frame Dependent Hexamer (H. sapiens)
ctcgac

SEQ ID NO: 292 - DNA, 6 bp, Artificial Sequence
  Rare Frame Dependent Hexamer (H. sapiens)
agtaga

SEQ ID NO: 293 - DNA, 6 bp, Artificial Sequence
  Rare Frame Dependent Hexamer (H. sapiens)
ttcgca

SEQ ID NO: 294 - DNA, 6 bp, Artificial Sequence
  Rare Frame Dependent Hexamer (H. sapiens)
ttcggt

SEQ ID NO: 295 - DNA, 6 bp, Artificial Sequence
  Rare Frame Dependent Hexamer (H. sapiens)
agtagg

SEQ ID NO: 296 - DNA, 6 bp, Artificial Sequence
  Rare Frame Dependent Hexamer (H. sapiens)
gatagc

SEQ ID NO: 297 - DNA, 6 bp, Artificial Sequence
  Rare Frame Dependent Hexamer (H. sapiens)
agcgaa

SEQ ID NO: 298 - DNA, 6 bp, Artificial Sequence
  Rare Frame Dependent Hexamer (H. sapiens)
tccggt

SEQ ID NO: 299 - DNA, 6 bp, Artificial Sequence
  Rare Frame Dependent Hexamer (H. sapiens)
gccggt

SEQ ID NO: 300 - DNA, 6 bp, Artificial Sequence
  Rare Frame Dependent Hexamer (H. sapiens)
tccgat

SEQ ID NO: 301 - DNA, 6 bp, Artificial Sequence
  Rare Frame Dependent Hexamer (H. sapiens)
accgcc

SEQ ID NO: 302 - DNA, 6 bp, Artificial Sequence
  Rare Frame Dependent Hexamer (H. sapiens)
gacgga

SEQ ID NO: 303 - DNA, 6 bp, Artificial Sequence
  Rare Frame Dependent Hexamer (H. sapiens)
ctcgat

SEQ ID NO: 304 - DNA, 6 bp, Artificial Sequence
  Rare Frame Dependent Hexamer (H. sapiens)
tgcgtt

SEQ ID NO: 305 - DNA, 6 bp, Artificial Sequence
  Rare Frame Dependent Hexamer (H. sapiens)
agtagc

SEQ ID NO: 306 - DNA, 6 bp, Artificial Sequence
  Rare Frame Dependent Hexamer (H. sapiens)
tacgct

SEQ ID NO: 307 - DNA, 6 bp, Artificial Sequence
  Rare Frame Dependent Hexamer (H. sapiens)
cccggc

SEQ ID NO: 308 - DNA, 6 bp, Artificial Sequence
  Rare Frame Dependent Hexamer (H. sapiens)
gtcgac

SEQ ID NO: 309 - DNA, 6 bp, Artificial Sequence
  Rare Frame Dependent Hexamer (H. sapiens)
gtcggt

SEQ ID NO: 310 - DNA, 6 bp, Artificial Sequence
  Rare Frame Dependent Hexamer (H. sapiens)
cccgcc

SEQ ID NO: 311 - DNA, 6 bp, Artificial Sequence
  Rare Frame Dependent Hexamer (H. sapiens)
ccatgc

SEQ ID NO: 312 - DNA, 6 bp, Artificial Sequence
  Rare Frame Dependent Hexamer (H. sapiens)
tctagc

SEQ ID NO: 313 - DNA, 6 bp, Artificial Sequence
  Rare Frame Dependent Hexamer (H. sapiens)
ggtaga

SEQ ID NO: 314 - DNA, 6 bp, Artificial Sequence
  Rare Frame Dependent Hexamer (H. sapiens)
tgcggt

SEQ ID NO: 315 - DNA, 6 bp, Artificial Sequence
  Rare Frame Dependent Hexamer (H. sapiens)
ggtagc

SEQ ID NO: 316 - DNA, 6 bp, Artificial Sequence
  Rare Frame Dependent Hexamer (H. sapiens)
tgcgaa

SEQ ID NO: 317 - DNA, 6 bp, Artificial Sequence
  Rare Frame Dependent Hexamer (H. sapiens)
atcgga

SEQ ID NO: 318 - DNA, 6 bp, Artificial Sequence
  Rare Frame Dependent Hexamer (H. sapiens)
cattgg

SEQ ID NO: 319 - DNA, 6 bp, Artificial Sequence
  Rare Frame Independent Hexamer (H. sapiens)
cgcgaa

SEQ ID NO: 320 - DNA, 6 bp, Artificial Sequence
  Rare Frame Independent Hexamer (H. sapiens)
tcgcga

SEQ ID NO: 321 - DNA, 6 bp, Artificial Sequence
  Rare Frame Independent Hexamer (H. sapiens)
cgatcg

SEQ ID NO: 322 - DNA, 6 bp, Artificial Sequence
  Rare Frame Independent Hexamer (H. sapiens)
cgaacg

SEQ ID NO: 323 - DNA, 6 bp, Artificial Sequence
  Rare Frame Independent Hexamer (H. sapiens)
acgcga

SEQ ID NO: 324 - DNA, 6 bp, Artificial Sequence
  Rare Frame Independent Hexamer (H. sapiens)
cgcgat

SEQ ID NO: 325 - DNA, 6 bp, Artificial Sequence
  Rare Frame Independent Hexamer (H. sapiens)
gcgaaa

SEQ ID NO: 326 - DNA, 6 bp, Artificial Sequence
  Rare Frame Independent Hexamer (H. sapiens)
gcgaac

SEQ ID NO: 327 - DNA, 6 bp, Artificial Sequence
  Rare Frame Independent Hexamer (H. sapiens)
cggtcg

SEQ ID NO: 328 - DNA, 6 bp, Artificial Sequence
  Rare Frame Independent Hexamer (H. sapiens)
cgcgta

SEQ ID NO: 329 - DNA, 6 bp, Artificial Sequence
  Rare Frame Independent Hexamer (H. sapiens)
cgcaat

SEQ ID NO: 330 - DNA, 6 bp, Artificial Sequence
  Rare Frame Independent Hexamer (H. sapiens)
cgtcga

SEQ ID NO: 331 - DNA, 6 bp, Artificial Sequence
  Rare Frame Independent Hexamer (H. sapiens)
ccggta

SEQ ID NO: 332 - DNA, 6 bp, Artificial Sequence
  Rare Frame Independent Hexamer (H. sapiens)
gttgcg

SEQ ID NO: 333 - DNA, 6 bp, Artificial Sequence
  Rare Frame Independent Hexamer (H. sapiens)
tcgatc

SEQ ID NO: 334 - DNA, 6 bp, Artificial Sequence
  Rare Frame Independent Hexamer (H. sapiens)
gcgatc

SEQ ID NO: 335 - DNA, 6 bp, Artificial Sequence
  Rare Frame Independent Hexamer (H. sapiens)
tcgcgt

SEQ ID NO: 336 - DNA, 6 bp, Artificial Sequence
  Rare Frame Independent Hexamer (H. sapiens)
tttcgc

SEQ ID NO: 337 - DNA, 6 bp, Artificial Sequence
  Rare Frame Independent Hexamer (H. sapiens)
acgatc

SEQ ID NO: 338 - DNA, 6 bp, Artificial Sequence
  Rare Frame Independent Hexamer (H. sapiens)
ttgcga

SEQ ID NO: 339 - DNA, 6 bp, Artificial Sequence
  Rare Frame Independent Hexamer (H. sapiens)
caatcg

SEQ ID NO: 340 - DNA, 6 bp, Artificial Sequence
  Rare Frame Independent Hexamer (H. sapiens)
cgacga

SEQ ID NO: 341 - DNA, 6 bp, Artificial Sequence
  Rare Frame Independent Hexamer (H. sapiens)
gtcgaa

SEQ ID NO: 342 - DNA, 6 bp, Artificial Sequence
  Rare Frame Independent Hexamer (H. sapiens)
cgcgac

SEQ ID NO: 343 - DNA, 6 bp, Artificial Sequence
  Rare Frame Independent Hexamer (H. sapiens)
ccgatc

SEQ ID NO: 344 - DNA, 6 bp, Artificial Sequence
  Rare Frame Independent Hexamer (H. sapiens)
tcgcaa

SEQ ID NO: 345 - DNA, 6 bp, Artificial Sequence
  Rare Frame Independent Hexamer (H. sapiens)
cgatca

SEQ ID NO: 346 - DNA, 6 bp, Artificial Sequence
  Rare Frame Independent Hexamer (H. sapiens)
tatacg

SEQ ID NO: 347 - DNA, 6 bp, Artificial Sequence
  Rare Frame Independent Hexamer (H. sapiens)
gcgata

SEQ ID NO: 348 - DNA, 6 bp, Artificial Sequence
  Rare Frame Independent Hexamer (H. sapiens)
cgtacg

SEQ ID NO: 349 - DNA, 6 bp, Artificial Sequence
  Rare Frame Independent Hexamer (H. sapiens)
ggcgaa

SEQ ID NO: 350 - DNA, 6 bp, Artificial Sequence
  Rare Frame Independent Hexamer (H. sapiens)
cgattg

SEQ ID NO: 351 - DNA, 6 bp, Artificial Sequence
  Rare Frame Independent Hexamer (H. sapiens)
tacgcg

SEQ ID NO: 352 - DNA, 6 bp, Artificial Sequence
  Rare Frame Independent Hexamer (H. sapiens)
cccccc

SEQ ID NO: 353 - DNA, 6 bp, Artificial Sequence
  Rare Frame Independent Hexamer (H. sapiens)
ttacgc

SEQ ID NO: 354 - DNA, 6 bp, Artificial Sequence
  Rare Frame Independent Hexamer (H. sapiens)
gcgcga

SEQ ID NO: 355 - DNA, 6 bp, Artificial Sequence
  Rare Frame Independent Hexamer (H. sapiens)
ctcgcg

SEQ ID NO: 356 - DNA, 6 bp, Artificial Sequence
  Rare Frame Independent Hexamer (H. sapiens)
gtcgcg

SEQ ID NO: 357 - DNA, 6 bp, Artificial Sequence
  Rare Frame Independent Hexamer (H. sapiens)
ttcgcg

SEQ ID NO: 358 - DNA, 6 bp, Artificial Sequence
  Rare Frame Independent Hexamer (H. sapiens)
ttttcg

SEQ ID NO: 359 - DNA, 6 bp, Artificial Sequence
  Rare Frame Independent Hexamer (H. sapiens)
gcgcaa

SEQ ID NO: 360 - DNA, 6 bp, Artificial Sequence
  Rare Frame Independent Hexamer (H. sapiens)
cgaaaa

SEQ ID NO: 361 - DNA, 6 bp, Artificial Sequence
  Rare Frame Independent Hexamer (H. sapiens)
gtcgat

SEQ ID NO: 362 - DNA, 6 bp, Artificial Sequence
  Rare Frame Independent Hexamer (H. sapiens)
cgcata

SEQ ID NO: 363 - DNA, 6 bp, Artificial Sequence
  Rare Frame Independent Hexamer (H. sapiens)
cgatcc

SEQ ID NO: 364 - DNA, 6 bp, Artificial Sequence
  Rare Frame Independent Hexamer (H. sapiens)
cgatct

SEQ ID NO: 365 - DNA, 6 bp, Artificial Sequence
  Rare Frame Independent Hexamer (H. sapiens)
cgcaac

SEQ ID NO: 366 - DNA, 6 bp, Artificial Sequence
  Rare Frame Independent Hexamer (H. sapiens)
cggtat

SEQ ID NO: 367 - DNA, 6 bp, Artificial Sequence
  Rare Frame Independent Hexamer (H. sapiens)
atacgc

SEQ ID NO: 368 - DNA, 6 bp, Artificial Sequence
  Rare Frame Independent Hexamer (H. sapiens)
attcgc

SEQ ID NO: 369 - DNA, 6 bp, Artificial Sequence
  Rare Frame Independent Hexamer (H. sapiens)
tcgaac

SEQ ID NO: 370 - DNA, 6 bp, Artificial Sequence
  Rare Frame Independent Hexamer (H. sapiens)
acggta

SEQ ID NO: 371 - DNA, 6 bp, Artificial Sequence
  Rare Frame Independent Hexamer (H. sapiens)
attgcg

SEQ ID NO: 372 - DNA, 6 bp, Artificial Sequence
  Rare Frame Independent Hexamer (H. sapiens)
aacgcg

SEQ ID NO: 373 - DNA, 6 bp, Artificial Sequence
  Rare Frame Independent Hexamer (H. sapiens)
tctcga

SEQ ID NO: 374 - DNA, 6 bp, Artificial Sequence
  Rare Frame Independent Hexamer (H. sapiens)
acgaac

SEQ ID NO: 375 - DNA, 6 bp, Artificial Sequence
  Rare Frame Independent Hexamer (H. sapiens)
acgata

SEQ ID NO: 376 - DNA, 6 bp, Artificial Sequence
  Rare Frame Independent Hexamer (H. sapiens)
cgatac

SEQ ID NO: 377 - DNA, 6 bp, Artificial Sequence
  Rare Frame Independent Hexamer (H. sapiens)
accggt

SEQ ID NO: 378 - DNA, 6 bp, Artificial Sequence
  Rare Frame Independent Hexamer (H. sapiens)
cgatta

SEQ ID NO: 379 - DNA, 6 bp, Artificial Sequence
  Rare Frame Independent Hexamer (H. sapiens)
ggtcga

SEQ ID NO: 380 - DNA, 6 bp, Artificial Sequence
  Rare Frame Independent Hexamer (H. sapiens)
gcgtac

SEQ ID NO: 381 - DNA, 6 bp, Artificial Sequence
  Rare Frame Independent Hexamer (H. sapiens)
gacgaa

SEQ ID NO: 382 - DNA, 6 bp, Artificial Sequence
  Rare Frame Independent Hexamer (H. sapiens)
gggggg

SEQ ID NO: 383 - DNA, 6 bp, Artificial Sequence
  Rare Frame Independent Hexamer (H. sapiens)
cgaacc

SEQ ID NO: 384 - DNA, 6 bp, Artificial Sequence
  Rare Frame Independent Hexamer (H. sapiens)
gtcgta

SEQ ID NO: 385 - DNA, 6 bp, Artificial Sequence
  Rare Frame Independent Hexamer (H. sapiens)
ataccg

SEQ ID NO: 386 - DNA, 6 bp, Artificial Sequence
  Rare Frame Independent Hexamer (H. sapiens)
cggtac

SEQ ID NO: 387 - DNA, 6 bp, Artificial Sequence
  Rare Frame Independent Hexamer (H. sapiens)
ggcgta

SEQ ID NO: 388 - DNA, 6 bp, Artificial Sequence
  Rare Frame Independent Hexamer (H. sapiens)
gcgatt

SEQ ID NO: 389 - DNA, 6 bp, Artificial Sequence
  Rare Frame Independent Hexamer (H. sapiens)
atcgcg

SEQ ID NO: 390 - DNA, 6 bp, Artificial Sequence
  Rare Frame Independent Hexamer (H. sapiens)
cgatat

SEQ ID NO: 391 - DNA, 6 bp, Artificial Sequence
  Rare Frame Independent Hexamer (H. sapiens)
cgaact

SEQ ID NO: 392 - DNA, 6 bp, Artificial Sequence
  Rare Frame Independent Hexamer (H. sapiens)
tcggta

SEQ ID NO: 393 - DNA, 6 bp, Artificial Sequence
  Rare Frame Independent Hexamer (H. sapiens)
acgcaa

SEQ ID NO: 394 - DNA, 6 bp, Artificial Sequence
  Rare Frame Independent Hexamer (H. sapiens)
taccgg

SEQ ID NO: 395 - DNA, 6 bp, Artificial Sequence
  Rare Frame Independent Hexamer (H. sapiens)
tttacg

SEQ ID NO: 396 - DNA, 6 bp, Artificial Sequence
  Rare Frame Independent Hexamer (H. sapiens)
ttgcgc

SEQ ID NO: 397 - DNA, 6 bp, Artificial Sequence
  Rare Frame Independent Hexamer (H. sapiens)
tcgacg

SEQ ID NO: 398 - DNA, 6 bp, Artificial Sequence
  Rare Frame Independent Hexamer (H. sapiens)
atttcg

SEQ ID NO: 399 - DNA, 6 bp, Artificial Sequence
  Rare Frame Independent Hexamer (H. sapiens)
gcggta

SEQ ID NO: 400 - DNA, 6 bp, Artificial Sequence
  Rare Frame Independent Hexamer (H. sapiens)
agcgta

SEQ ID NO: 401 - DNA, 6 bp, Artificial Sequence
  Rare Frame Independent Hexamer (H. sapiens)
gcgtat

SEQ ID NO: 402 - DNA, 6 bp, Artificial Sequence
  Rare Frame Independent Hexamer (H. sapiens)
ccgata

SEQ ID NO: 403 - DNA, 6 bp, Artificial Sequence
  Rare Frame Independent Hexamer (H. sapiens)
ccgaac

SEQ ID NO: 404 - DNA, 6 bp, Artificial Sequence
  Rare Frame Independent Hexamer (H. sapiens)
acgaaa

SEQ ID NO: 405 - DNA, 6 bp, Artificial Sequence
  Rare Frame Independent Hexamer (H. sapiens)
gtcgac

SEQ ID NO: 406 - DNA, 6 bp, Artificial Sequence
  Rare Frame Independent Hexamer (H. sapiens)
attacg

SEQ ID NO: 407 - DNA, 6 bp, Artificial Sequence
  Rare Frame Independent Hexamer (H. sapiens)
tttcga

SEQ ID NO: 408 - DNA, 6 bp, Artificial Sequence
  Rare Frame Independent Hexamer (H. sapiens)
catacg

SEQ ID NO: 409 - DNA, 6 bp, Artificial Sequence
  Rare Frame Independent Hexamer (H. sapiens)
cgaaac

SEQ ID NO: 410 - DNA, 6 bp, Artificial Sequence
  Rare Frame Independent Hexamer (H. sapiens)
cgaaca

SEQ ID NO: 411 - DNA, 6 bp, Artificial Sequence
  Rare Frame Independent Hexamer (H. sapiens)
cgtata

SEQ ID NO: 412 - DNA, 6 bp, Artificial Sequence
  Rare Frame Independent Hexamer (H. sapiens)
acgcgt

SEQ ID NO: 413 - DNA, 6 bp, Artificial Sequence
  Rare Frame Independent Hexamer (H. sapiens)
gacgta

SEQ ID NO: 414 - DNA, 6 bp, Artificial Sequence
  Rare Frame Independent Hexamer (H. sapiens)
ctatcg

SEQ ID NO: 415 - DNA, 6 bp, Artificial Sequence
  Rare Frame Independent Hexamer (H. sapiens)
ttgcgt

SEQ ID NO: 416 - DNA, 6 bp, Artificial Sequence
  Rare Frame Independent Hexamer (H. sapiens)
acgatt

SEQ ID NO: 417 - DNA, 6 bp, Artificial Sequence
  Rare Frame Independent Hexamer (H. sapiens)
tcgatt

SEQ ID NO: 418 - DNA, 6 bp, Artificial Sequence
  Rare Frame Independent Hexamer (H. sapiens)
ccgcga

SEQ ID NO: 419 - DNA, 2280 bp, Artificial Sequence
  Influenza PB2 increased rare FD hexamer
atggagagaataaaggagctcagagacctgatgagtcagtcaagaacaagagaaatcctgactaaaacaacagtggaccacatggctatcattaagaaatatacatcaggcagacaggagaagaatccagctttgagaatgaagtggatgatggctatgagatatccaataacagctgacaagagaataatggacatgattccagagagaaatgaacaaggacagaccctctggagcaagacaaatgacgctggaagtgacagagtgatggtgagcccactggctgtgacatggtggaatagaaatggacctacaacttcaacagtgcactacccaaaggtttacaagacatacttcgagaaggtggagaggctgaagcatggaacctttggaccagttcacttcagaaaccaagtaaaaatcagaagaagagtggacacaaatccaggacatgcagatttgtcagcaaaggaggcccaggatgtcattatggaggtggtcttcccaaatgaagttggagcaagaattctgacatcagagagccagctggctattacaaaggagaagaaggaggagctgcaggactgtaaaatcgccccactgatggtggcttatatgctggagagagagctggtgaggaagacaaggttcctcccagtggcaggaggaactggaagcgtctacatcgaggtgctccatctgacacaaggaacctgctgggagcagatgtacaccccaggaggagaggtgagaaatgatgatgtggaccagtcactcattatcgctgctagaaatattgtgagaagagcagcagtgtcagctgaccctttggcctctttgctggagatgtgccatagcacacagataggaggagtcagaatggtggacatccttagacaaaacccaacagaggagcaggctgtggacatttgcaaagcagcaattggactgagaatctcatcttcattcagctttggaggatttacctttaaaaggacatcaggaagctcagtgaagaaggaggaggaagtcctgactggaaaccttcagacactgaagatcagagttcatgaaggatatgaagagttcaccatggtgggaagaagagctacagccattttgaggaaggctacaagaaggctgattcagctcattgtgtcaggaagagatgagcagagcatcgccgaggccattatagtggcaatggtcttctcacaggaagattgcatgattaaagcagtgagaggagacttgaacttcgtgaatagagcaaaccagagactcaacccaatgcaccagctgcttcggcacttccagaaggatgcaaaggtgctcttccagaactggggaattgagtccattgataatgtgatgggaatgattggaattttgccagacatgacaccaagtacagagatgagcctgagaggaataagagtcagcaaaatgggagtggatgaatattcctctacagagagagtggtggtcagcatcgacagattcctcagagtcagagaccagagaggaaatgttctcctgtctccagaggaagtgagtgaaacccaaggaacagagaagttgacaataacttacagctcttcaatgatgtgggaaatcaacggcccagagtcagtattggtgaatacttaccagtggattatcagaaattgggaaatagtgaagattcagtggtcacaagacccaacaatgctctacaacaagatggagtttgagccattccagagcctggtcccaaaggccatcagaagtagatattctgggttcgtcaggaccctcttccagcagatgagagatgtgctggggacctttgacacagtgcagataattaaacttctgccatttgcagcagcaccaccagagcagagtagaatgcagttcagtagcctcactgtgaatgtgagagggtctgggctcagaattttggtgagaggaaattcaccagtgtttaattataataaagcaacaaagagactgacagtgctgggaaaagatgctggggctttgacagaggacccagatgaaggaacttctggagtggagtcagcagtgctcagaggcttcctcattctgggaaaggaagacaagagatatggaccagcactttccataaatgaactcagtaaccttgccaaaggagagaaggcaaatgtgctaattggacaaggagatgtggtgctggtgatgaagaggaagagagatagtagcatcttgacagatagccagacagcaacaaagagaatcagaatggctataaactag